SEQUENCE LISTING (212 sequences)

SEQ ID NO: 1 - protein, 11 aa, Homo sapiens
SVLGPILIIPK

SEQ ID NO: 2 - protein, 9 aa, Homo sapiens
DMFGIRFKR

SEQ ID NO: 3 - protein, 9 aa, Homo sapiens
EYNAEFGRR

SEQ ID NO: 4 - protein, 9 aa, Homo sapiens
FPEDFSILF

SEQ ID NO: 5 - protein, 9 aa, Homo sapiens
GEAVGFQPP

SEQ ID NO: 6 - protein, 10 aa, Homo sapiens
IYLIPEPVLF

SEQ ID NO: 7 - protein, 9 aa, Homo sapiens
LEISFISPL

SEQ ID NO: 8 - protein, 9 aa, Homo sapiens
RSMRVGVTR

SEQ ID NO: 9 - protein, 9 aa, Homo sapiens
SMRSTVVSR

SEQ ID NO: 10 - protein, 9 aa, Homo sapiens
SVADLLSSF

SEQ ID NO: 11 - protein, 10 aa, Homo sapiens
AETDITETSI

SEQ ID NO: 12 - protein, 9 aa, Homo sapiens
DIIEYDYEY

SEQ ID NO: 13 - protein, 8 aa, Homo sapiens
EEIFTEEY

SEQ ID NO: 14 - protein, 8 aa, Homo sapiens
EEQYIAQF

SEQ ID NO: 15 - protein, 9 aa, Homo sapiens
ELFQHVSLM

SEQ ID NO: 16 - protein, 9 aa, Homo sapiens
GAYDKARYL

SEQ ID NO: 17 - protein, 10 aa, Homo sapiens
GTHVWVGLYK

SEQ ID NO: 18 - protein, 8 aa, Homo sapiens
HQFTEKVI

SEQ ID NO: 19 - protein, 9 aa, Homo sapiens
KLKQDTTQK

SEQ ID NO: 20 - protein, 9 aa, Homo sapiens
LELFQHVSL

SEQ ID NO: 21 - protein, 9 aa, Homo sapiens
MAVNIVLQL

SEQ ID NO: 22 - protein, 9 aa, Homo sapiens
MLWKLVENV

SEQ ID NO: 23 - protein, 9 aa, Homo sapiens
QAWGAGLAY

SEQ ID NO: 24 - protein, 9 aa, Homo sapiens
SEADLQAEV

SEQ ID NO: 25 - protein, 10 aa, Homo sapiens
SLPDNVFSNL

SEQ ID NO: 26 - protein, 9 aa, Homo sapiens
SQGQIFSKF

SEQ ID NO: 27 - protein, 9 aa, Homo sapiens
SRLELFQHV

SEQ ID NO: 28 - protein, 9 aa, Homo sapiens
SSQGQIFSK

SEQ ID NO: 29 - protein, 9 aa, Homo sapiens
SVKERESER

SEQ ID NO: 30 - protein, 9 aa, Homo sapiens
VEEIFTEEY

SEQ ID NO: 31 - protein, 11 aa, Homo sapiens
VHDPDYAKILL

SEQ ID NO: 32 - protein, 9 aa, Homo sapiens
VPWEDKISI

SEQ ID NO: 33 - protein, 8 aa, Homo sapiens
VTTLALTF

SEQ ID NO: 34 - protein, 8 aa, Homo sapiens
WPMHTPLL

SEQ ID NO: 35 - protein, 9 aa, Homo sapiens
WPMHTPLLL

SEQ ID NO: 36 - protein, 9 aa, Homo sapiens
EADPTGHSY

SEQ ID NO: 37 - protein, 9 aa, Homo sapiens
EVDPASNTY

SEQ ID NO: 38 - protein, 9 aa, Homo sapiens
EVDPTSHSY

SEQ ID NO: 39 - protein, 11 aa, Homo sapiens
EVYDGREHSAY

SEQ ID NO: 40 - protein, 9 aa, Homo sapiens
FVQENYLEY

SEQ ID NO: 41 - protein, 9 aa, Homo sapiens
GEALGLVGA

SEQ ID NO: 42 - protein, 9 aa, Homo sapiens
LVQEKYLEY

SEQ ID NO: 43 - protein, 9 aa, Homo sapiens
TFLQRKVLR

SEQ ID NO: 44 - protein, 9 aa, Homo sapiens
DETLTALQF

SEQ ID NO: 45 - protein, 10 aa, Homo sapiens
EVFEGDIQQF

SEQ ID NO: 46 - protein, 9 aa, Homo sapiens
EVKSITKER

SEQ ID NO: 47 - protein, 9 aa, Homo sapiens
FAFEKLIQF

SEQ ID NO: 48 - protein, 9 aa, Homo sapiens
HHNDLVFKF

SEQ ID NO: 49 - protein, 9 aa, Homo sapiens
ITDDQLLAL

SEQ ID NO: 50 - protein, 9 aa, Homo sapiens
LEIENLNLA

SEQ ID NO: 51 - protein, 9 aa, Homo sapiens
MPLKHYLLL

SEQ ID NO: 52 - protein, 9 aa, Homo sapiens
MVFSKLVLR

SEQ ID NO: 53 - protein, 9 aa, Homo sapiens
QLLALLPSL

SEQ ID NO: 54 - protein, 9 aa, Homo sapiens
TTLENLPQK

SEQ ID NO: 55 - protein, 9 aa, Homo sapiens
VEAPHLPSF

SEQ ID NO: 56 - protein, 8 aa, Homo sapiens
DELAHFLL

SEQ ID NO: 57 - protein, 8 aa, Homo sapiens
FPDLETSF

SEQ ID NO: 58 - protein, 9 aa, Homo sapiens
EELSVLEVF

SEQ ID NO: 59 - protein, 9 aa, Homo sapiens
EELSVMEVY

SEQ ID NO: 60 - protein, 8 aa, Homo sapiens
FPDLESEF

SEQ ID NO: 61 - protein, 9 aa, Homo sapiens
YEFLWGSKA

SEQ ID NO: 62 - protein, 9 aa, Homo sapiens
AETLSPLSL

SEQ ID NO: 63 - protein, 9 aa, Homo sapiens
AEVERSPEP

SEQ ID NO: 64 - protein, 9 aa, Homo sapiens
ALSSVPLIY

SEQ ID NO: 65 - protein, 8 aa, Homo sapiens
AQLHIPVV

SEQ ID NO: 66 - protein, 8 aa, Homo sapiens
DGSHALVL

SEQ ID NO: 67 - protein, 9 aa, Homo sapiens
DLSPGVQGY

SEQ ID NO: 68 - protein, 9 aa, Homo sapiens
DVSGVSSFW

SEQ ID NO: 69 - protein, 9 aa, Homo sapiens
EAGSLHLQR

SEQ ID NO: 70 - protein, 9 aa, Homo sapiens
ELISELSKI

SEQ ID NO: 71 - protein, 9 aa, Homo sapiens
ETQIKTTTR

SEQ ID NO: 72 - protein, 9 aa, Homo sapiens
EVERSPEPR

SEQ ID NO: 73 - protein, 9 aa, Homo sapiens
EVRGGGITR

SEQ ID NO: 74 - protein, 9 aa, Homo sapiens
EVVGLLESR

SEQ ID NO: 75 - protein, 8 aa, Homo sapiens
FEFLFTNL

SEQ ID NO: 76 - protein, 9 aa, Homo sapiens
FPHSLLSVL

SEQ ID NO: 77 - protein, 9 aa, Homo sapiens
GEDLEKVTI

SEQ ID NO: 78 - protein, 9 aa, Homo sapiens
GEKEILITV

SEQ ID NO: 79 - protein, 9 aa, Homo sapiens
GIRPPTSVR

SEQ ID NO: 80 - protein, 9 aa, Homo sapiens
GQIKKLLQM

SEQ ID NO: 81 - protein, 8 aa, Homo sapiens
IDSISWLL

SEQ ID NO: 82 - protein, 8 aa, Homo sapiens
IDSLSWLL

SEQ ID NO: 83 - protein, 10 aa, Homo sapiens
IEMNSILFQM

SEQ ID NO: 84 - protein, 9 aa, Homo sapiens
IISSNILSV

SEQ ID NO: 85 - protein, 9 aa, Homo sapiens
ILLPQPPGV

SEQ ID NO: 86 - protein, 9 aa, Homo sapiens
ILTEQEEIL

SEQ ID NO: 87 - protein, 8 aa, Homo sapiens
IPLLSQIY

SEQ ID NO: 88 - protein, 8 aa, Homo sapiens
ISAISALW

SEQ ID NO: 89 - protein, 9 aa, Homo sapiens
ISLLPISEK

SEQ ID NO: 90 - protein, 9 aa, Homo sapiens
ISSAFTLFL

SEQ ID NO: 91 - protein, 8 aa, Homo sapiens
ITATSASW

SEQ ID NO: 92 - protein, 9 aa, Homo sapiens
IYLVEPLKF

SEQ ID NO: 93 - protein, 8 aa, Homo sapiens
LESIGYVL

SEQ ID NO: 94 - protein, 9 aa, Homo sapiens
LISSNILSV

SEQ ID NO: 95 - protein, 9 aa, Homo sapiens
LLNSQWVSK

SEQ ID NO: 96 - protein, 8 aa, Homo sapiens
LPLLVNII

SEQ ID NO: 97 - protein, 9 aa, Homo sapiens
LPSPSIVSL

SEQ ID NO: 98 - protein, 8 aa, Homo sapiens
LRNLHNVL

SEQ ID NO: 99 - protein, 9 aa, Homo sapiens
LTSGWLLSK

SEQ ID NO: 100 - protein, 8 aa, Homo sapiens
NEALAAIL

SEQ ID NO: 101 - protein, 8 aa, Homo sapiens
NETAAYKL

SEQ ID NO: 102 - protein, 8 aa, Homo sapiens
NQTPHILI

SEQ ID NO: 103 - protein, 8 aa, Homo sapiens
QDSLVLNL

SEQ ID NO: 104 - protein, 8 aa, Homo sapiens
QPAAASSL

SEQ ID NO: 105 - protein, 8 aa, Homo sapiens
QQQKLLKI

SEQ ID NO: 106 - protein, 9 aa, Homo sapiens
RFIDLTSTF

SEQ ID NO: 107 - protein, 9 aa, Homo sapiens
RLLPLEVLL

SEQ ID NO: 108 - protein, 9 aa, Homo sapiens
RSFTMRSSR

SEQ ID NO: 109 - protein, 11 aa, Homo sapiens
RTLAVSVTALK

SEQ ID NO: 110 - protein, 9 aa, Homo sapiens
SIMHSFILK

SEQ ID NO: 111 - protein, 8 aa, Homo sapiens
SLFNLFPR

SEQ ID NO: 112 - protein, 8 aa, Homo sapiens
SPAEKQII

SEQ ID NO: 113 - protein, 9 aa, Homo sapiens
SPWIILRDI

SEQ ID NO: 114 - protein, 9 aa, Homo sapiens
SQLISLQTL

SEQ ID NO: 115 - protein, 9 aa, Homo sapiens
SSDPPTSTL

SEQ ID NO: 116 - protein, 8 aa, Homo sapiens
SSYLVSQL

SEQ ID NO: 117 - protein, 9 aa, Homo sapiens
SVVSLVVQK

SEQ ID NO: 118 - protein, 9 aa, Homo sapiens
TESGINVTL

SEQ ID NO: 119 - protein, 9 aa, Homo sapiens
TILESSLTL

SEQ ID NO: 120 - protein, 9 aa, Homo sapiens
TLFNFLVSL

SEQ ID NO: 121 - protein, 9 aa, Homo sapiens
TLNDLTTSW

SEQ ID NO: 122 - protein, 9 aa, Homo sapiens
TLSGWLLSK

SEQ ID NO: 123 - protein, 8 aa, Homo sapiens
TMLKLVLI

SEQ ID NO: 124 - protein, 9 aa, Homo sapiens
TPRHLIVRF

SEQ ID NO: 125 - protein, 9 aa, Homo sapiens
TTNPSIKKR

SEQ ID NO: 126 - protein, 8 aa, Homo sapiens
VDNSTISI

SEQ ID NO: 127 - protein, 9 aa, Homo sapiens
VEVDRLLEV

SEQ ID NO: 128 - protein, 8 aa, Homo sapiens
VGFILPSV

SEQ ID NO: 129 - protein, 8 aa, Homo sapiens
VQFIIYQL

SEQ ID NO: 130 - protein, 9 aa, Homo sapiens
VRSSIILTY

SEQ ID NO: 131 - protein, 9 aa, Homo sapiens
VSAQAGVQW

SEQ ID NO: 132 - protein, 9 aa, Homo sapiens
VYLSSLALL

SEQ ID NO: 133 - protein, 9 aa, Homo sapiens
WLMPVLPAL

SEQ ID NO: 134 - protein, 9 aa, Homo sapiens
YVAQAGLEI

SEQ ID NO: 135 - protein, 9 aa, Homo sapiens
ITKVVFPGR

SEQ ID NO: 136 - protein, 9 aa, Homo sapiens
DEDLFTDLY

SEQ ID NO: 137 - protein, 9 aa, Homo sapiens
WETRLHNLW

SEQ ID NO: 138 - protein, 11 aa, Homo sapiens
EVFEGREDSVF

SEQ ID NO: 139 - protein, 9 aa, Homo sapiens
DEFNGVPSY

SEQ ID NO: 140 - protein, 10 aa, Homo sapiens
DEQTPIHISW

SEQ ID NO: 141 - protein, 10 aa, Homo sapiens
DVITIIDRSF

SEQ ID NO: 142 - protein, 9 aa, Homo sapiens
EEYLVPQAF

SEQ ID NO: 143 - protein, 10 aa, Homo sapiens
FPDVPDKENF

SEQ ID NO: 144 - protein, 9 aa, Homo sapiens
GSLGHLQNR

SEQ ID NO: 145 - protein, 9 aa, Homo sapiens
SYLKAVFNL

SEQ ID NO: 146 - protein, 9 aa, Homo sapiens
TLDSYLKAV

SEQ ID NO: 147 - protein, 9 aa, Homo sapiens
TLFDYEVRL

SEQ ID NO: 148 - protein, 9 aa, Homo sapiens
TQFFIPYTI

SEQ ID NO: 149 - protein, 8 aa, Homo sapiens
YEDLSQKY

SEQ ID NO: 150 - protein, 9 aa, Homo sapiens
YPVKEFEVY

SEQ ID NO: 151 - DNA, 33 bp, Homo sapiens
  source 1..33: mol_type = unassigned DNA
agtgtactcgggccaatattgattataccaaag

SEQ ID NO: 152 - DNA, 27 bp, Homo sapiens
  source 1..27: mol_type = unassigned DNA
gatatgtttggaataaggttcaaaaga

SEQ ID NO: 153 - DNA, 27 bp, Homo sapiens
  source 1..27: mol_type = unassigned DNA
gaatacaacgcagaatttggccgaagg

SEQ ID NO: 154 - DNA, 27 bp, Homo sapiens
  source 1..27: mol_type = unassigned DNA
ttcccagaagacttttcaatactattt

SEQ ID NO: 155 - DNA, 27 bp, Homo sapiens
  source 1..27: mol_type = unassigned DNA
ggcgaggctgtgggctttcaaccacca

SEQ ID NO: 156 - DNA, 30 bp, Homo sapiens
  source 1..30: mol_type = unassigned DNA
atttacctgattccagagcctgtgttgttt

SEQ ID NO: 157 - DNA, 27 bp, Homo sapiens
  source 1..27: mol_type = unassigned DNA
ttggagataagtttcatttcaccccta

SEQ ID NO: 158 - DNA, 27 bp, Homo sapiens
  source 1..27: mol_type = unassigned DNA
cgcagtatgcgggtgggagtgacccga

SEQ ID NO: 159 - DNA, 27 bp, Homo sapiens
  source 1..27: mol_type = unassigned DNA
tctatgcgctccacggtggtgtctcgc

SEQ ID NO: 160 - DNA, 27 bp, Homo sapiens
  source 1..27: mol_type = unassigned DNA
tctgtcgccgacctgctgtcctccttc

SEQ ID NO: 161 - DNA, 30 bp, Homo sapiens
  source 1..30: mol_type = unassigned DNA
gcagaaactgatattacagaaacaagcata

SEQ ID NO: 162 - DNA, 27 bp, Homo sapiens
  source 1..27: mol_type = unassigned DNA
gatataatcgaatatgactatgagtat

SEQ ID NO: 163 - DNA, 24 bp, Homo sapiens
  source 1..24: mol_type = unassigned DNA
gaagaaatatttactgaagaatat

SEQ ID NO: 164 - DNA, 24 bp, Homo sapiens
  source 1..24: mol_type = unassigned DNA
gaagagcagtatatcgcccagttc

SEQ ID NO: 165 - DNA, 27 bp, Homo sapiens
  source 1..27: mol_type = unassigned DNA
gagctctttcaacatgtctccctgatg

SEQ ID NO: 166 - DNA, 27 bp, Homo sapiens
  source 1..27: mol_type = unassigned DNA
ggcgcatatgacaaggcccgatacctc

SEQ ID NO: 167 - DNA, 30 bp, Homo sapiens
  source 1..30: mol_type = unassigned DNA
gggactcatgtttgggtaggcctgtataag

SEQ ID NO: 168 - DNA, 24 bp, Homo sapiens
  source 1..24: mol_type = unassigned DNA
catcagttcacagagaaagtaatc

SEQ ID NO: 169 - DNA, 27 bp, Homo sapiens
  source 1..27: mol_type = unassigned DNA
aagctaaaacaagatactactcagaaa

SEQ ID NO: 170 - DNA, 27 bp, Homo sapiens
  source 1..27: mol_type = unassigned DNA
ctggagctctttcaacatgtctccctg

SEQ ID NO: 171 - DNA, 27 bp, Homo sapiens
  source 1..27: mol_type = unassigned DNA
atggctgtcaacatcgtgctgcagctg

SEQ ID NO: 172 - DNA, 27 bp, Homo sapiens
  source 1..27: mol_type = unassigned DNA
atgctctggaagcttgtggagaatgtc

SEQ ID NO: 173 - DNA, 27 bp, Homo sapiens
  source 1..27: mol_type = unassigned DNA
caagcctggggtgcagggttggcctac

SEQ ID NO: 174 - DNA, 27 bp, Homo sapiens
  source 1..27: mol_type = unassigned DNA
tctgaagcagatctccaggctgaagtg

SEQ ID NO: 175 - DNA, 30 bp, Homo sapiens
  source 1..30: mol_type = unassigned DNA
tctctacccgacaatgtcttcagcaacctc

SEQ ID NO: 176 - DNA, 27 bp, Homo sapiens
  source 1..27: mol_type = unassigned DNA
tctcaaggccaaatcttttctaaattt

SEQ ID NO: 177 - DNA, 27 bp, Homo sapiens
  source 1..27: mol_type = unassigned DNA
tcacgtctggagctctttcaacatgtc

SEQ ID NO: 178 - DNA, 27 bp, Homo sapiens
  source 1..27: mol_type = unassigned DNA
agctctcaaggccaaatcttttctaaa

SEQ ID NO: 179 - DNA, 27 bp, Homo sapiens
  source 1..27: mol_type = unassigned DNA
tctgtgaaagaaagagagagtgagaga

SEQ ID NO: 180 - DNA, 27 bp, Homo sapiens
  source 1..27: mol_type = unassigned DNA
gttgaagaaatatttactgaagaatat

SEQ ID NO: 181 - DNA, 33 bp, Homo sapiens
  source 1..33: mol_type = unassigned DNA
gtccatgacccagactatgccaagattctcctg

SEQ ID NO: 182 - DNA, 27 bp, Homo sapiens
  source 1..27: mol_type = unassigned DNA
gttccctgggaggataaaatttccatt

SEQ ID NO: 183 - DNA, 24 bp, Homo sapiens
  source 1..24: mol_type = unassigned DNA
gtaacaaccctcgcattgaccttc

SEQ ID NO: 184 - DNA, 24 bp, Homo sapiens
  source 1..24: mol_type = unassigned DNA
tggcccatgcacaccccactgctg

SEQ ID NO: 185 - DNA, 27 bp, Homo sapiens
  source 1..27: mol_type = unassigned DNA
tggcccatgcacaccccactgctgctg

SEQ ID NO: 186 - DNA, 27 bp, Homo sapiens
  source 1..27: mol_type = unassigned DNA
gaagcagaccccaccggccactcctat

SEQ ID NO: 187 - DNA, 27 bp, Homo sapiens
  source 1..27: mol_type = unassigned DNA
gaagtggaccccgccagcaacacctac

SEQ ID NO: 188 - DNA, 27 bp, Homo sapiens
  source 1..27: mol_type = unassigned DNA
gaagtggaccccactagccactcctat

SEQ ID NO: 189 - DNA, 33 bp, Homo sapiens
  source 1..33: mol_type = unassigned DNA
gaggtgtatgatgggagggagcacagtgcctat

SEQ ID NO: 190 - DNA, 27 bp, Homo sapiens
  source 1..27: mol_type = unassigned DNA
ttcgtgcaggaaaactacctggagtac

SEQ ID NO: 191 - DNA, 27 bp, Homo sapiens
  source 1..27: mol_type = unassigned DNA
ggagaggccctgggcttggtgggtgcg

SEQ ID NO: 192 - DNA, 27 bp, Homo sapiens
  source 1..27: mol_type = unassigned DNA
ttggtgcaggaaaagtacctggagtac

SEQ ID NO: 193 - DNA, 27 bp, Homo sapiens
  source 1..27: mol_type = unassigned DNA
acctttttgcagaggaaggtactgagg

SEQ ID NO: 194 - DNA, 27 bp, Homo sapiens
  source 1..27: mol_type = unassigned DNA
gatgaaacacttactgctctccagttt

SEQ ID NO: 195 - DNA, 30 bp, Homo sapiens
  source 1..30: mol_type = unassigned DNA
gaagtttttgagggggacattcagcagttt

SEQ ID NO: 196 - DNA, 27 bp, Homo sapiens
  source 1..27: mol_type = unassigned DNA
gaagtgaaatctataaccaaagaaaga

SEQ ID NO: 197 - DNA, 27 bp, Homo sapiens
  source 1..27: mol_type = unassigned DNA
tttgcctttgagaagctcatccaattt

SEQ ID NO: 198 - DNA, 27 bp, Homo sapiens
  source 1..27: mol_type = unassigned DNA
catcacaacgacctggttttcaaattc

SEQ ID NO: 199 - DNA, 27 bp, Homo sapiens
  source 1..27: mol_type = unassigned DNA
atcacggatgatcagctccttgccctc

SEQ ID NO: 200 - DNA, 27 bp, Homo sapiens
  source 1..27: mol_type = unassigned DNA
ttagaaatagagaatttaaatttggct

SEQ ID NO: 201 - DNA, 27 bp, Homo sapiens
  source 1..27: mol_type = unassigned DNA
atgccactgaagcattatctccttttg

SEQ ID NO: 202 - DNA, 27 bp, Homo sapiens
  source 1..27: mol_type = unassigned DNA
atggtattcagcaaattggtgttgagg

SEQ ID NO: 203 - DNA, 27 bp, Homo sapiens
  source 1..27: mol_type = unassigned DNA
cagctccttgccctcctgccttccctg

SEQ ID NO: 204 - DNA, 27 bp, Homo sapiens
  source 1..27: mol_type = unassigned DNA
accacactggaaaacctgccccagaag

SEQ ID NO: 205 - DNA, 27 bp, Homo sapiens
  source 1..27: mol_type = unassigned DNA
gtggaagcccctcatctgccttcattc

SEQ ID NO: 206 - DNA, 24 bp, Homo sapiens
  source 1..24: mol_type = unassigned DNA
gatgagttggctcattttctgctc

SEQ ID NO: 207 - DNA, 24 bp, Homo sapiens
  source 1..24: mol_type = unassigned DNA
tttcctgacctggagacgagcttc

SEQ ID NO: 208 - DNA, 27 bp, Homo sapiens
  source 1..27: mol_type = unassigned DNA
gaggagctgagtgtgttagaggtgttt

SEQ ID NO: 209 - DNA, 27 bp, Homo sapiens
  source 1..27: mol_type = unassigned DNA
gaggagctgagtgtgatggaggtgtat

SEQ ID NO: 210 - DNA, 24 bp, Homo sapiens
  source 1..24: mol_type = unassigned DNA
ttccctgacctggagtctgagttc

SEQ ID NO: 211 - DNA, 27 bp, Homo sapiens
  source 1..27: mol_type = unassigned DNA
tacgagttcctgtggggttccaaggcc

SEQ ID NO: 212 - protein, 10 aa, Homo sapiens
ELAGIGILTV

(19) United States
(12) Patent Application Publication
(10) Pub. No.: US 2026/0048105 A1
(43) Pub. Date: Feb. 19, 2026

(54) NOVEL ANTIGENS FOR CANCER AND USES THEREOF

(71) Applicant: UNIVERSITÉ DE MONTRÉAL, Montréal (CA)

(72) Inventors: Claude PERREAULT, Montréal (CA); Pierre THIBAULT, Montreal (CA); Eralda KINA, Montréal (CA)

(21) Appl. No.: 19/101,783

(22) PCT Filed: Aug. 4, 2023
(86) PCT No.: PCT/CA2023/051050

§ 371 Date: Feb. 6, 2025

Related U.S. Application Data
(60) Provisional application No. 63/370,697, filed on Aug. 8, 2022.

(51) Int. Cl. A61K 39/00; A61K 38/00; A61K 40/31; A61K 40/32; A61K 40/42; A61P 35/00; C07K 7/06; C07K 14/00; C07K 14/725; C07K 14/74; C07K 16/28

(52) U.S. Cl. CPC A61K 39/0011; A61K 40/31; A61K 40/32; A61K 40/421; A61P 35/00; C07K 7/06; C07K 14/001; C07K 14/7051; C07K 14/70539; C07K 16/2833; A61K 38/00; A61K 2039/53; A61K 2039/55555; A61K 2039/572; A61K 2239/13

(57) ABSTRACT

Breast cancer is now the most prevalent cancer worldwide, and despite therapeutical advances in the last decades, metastatic breast cancer remains an incurable disease. Novel tumor-specific antigens (TSAs) and tumor-associated antigens (TAAs) expressed by breast tumor cells are described herein. Synthetic long peptides, nucleic acids, compositions, cells, TCRs, antibodies and vaccines derived from these TSAs and TAAs are described. The use of the TSAs/TAAs, nucleic acids, compositions, antibodies, cells and vaccines for the prevention or treatment of breast cancer, including triple-negative breast cancer (TNBC), is also described.

### CROSS REFERENCE TO RELATED APPLICATIONS

The present application claims the benefit of U.S. provisional patent application No. 63/370,697, filed on Aug. 8, 2022, which is incorporated herein by reference.

### SEQUENCE LISTING

A sequence listing is submitted herewith as an XML file named 17971-00066-AD.xml, that was created on Aug. 2, 2023, and having a size of ˜181 kilobytes. The content of the aforementioned file is hereby incorporated by reference in its entirety.

### TECHNICAL FIELD

The present invention generally relates to the field of cancer, and more particularly to the treatment of cancer such as breast cancer.

### BACKGROUND ART

According to recent global statistics, breast cancer is now the most prevalent cancer worldwide, outranking lung cancer (1). Despite therapeutical advances in the last decades, metastatic breast cancer remains an incurable disease. From an immunological standpoint, breast cancer tumors fall into two main categories (2). Hormone-receptor-positive breast cancer (HR⁺) is considered an immunologically “cold” cancer and has not benefited from recent advances in immunotherapy (3). In contrast, the triple-negative breast cancer subtype (TNBC) is immunologically “hot,” as evidenced by high levels of leukocytic infiltration and responsiveness to immune checkpoint blockade (ICB) (4). In fact, following the IMPassion130 trial, the addition of ICB to chemotherapy is the new standard of care for TNBC tumors with PD-L1 expression (5). High tumor mutational burden (TMB) has previously been used as a biomarker for response to ICB in multiple cancers (6). However, it is only present in 5% of breast cancer tumors and is not correlated with CD8⁺ T cell infiltration (6,7). Therefore, the cause of the differential immunogenicity of HR⁺ and TNBC tumors remains elusive.

There is thus a need for the development of novel approaches for the prevention or treatment of breast cancer.

The present description refers to a number of documents, the content of which is herein incorporated by reference in their entirety.

### SUMMARY

In various aspects and embodiments, the present disclosure provides the following items 1 to 67:
1. A tumor antigen peptide (TAP) comprising or consisting of one of the following amino acid sequences:

| Peptide sequence | SEQ ID NO: |
|---|---|
| SVLGPILIIPK | 1 |
| DMFGIRFKR | 2 |
| EYNAEFGRR | 3 |
| FPEDFSILF | 4 |
| GEAVGFQPP | 5 |
| IYLIPEPVLF | 6 |
| LEISFISPL | 7 |
| RSMRVGVTR | 8 |
| SMRSTVVSR | 9 |
| SVADLLSSF | 10 |
| AETDITETSI | 11 |
| DIIEYDYEY | 12 |
| EEIFTEEY | 13 |
| EEQYIAQF | 14 |
| ELFQHVSLM | 15 |
| GAYDKARYL | 16 |
| GTHVWVGLYK | 17 |
| HQFTEKVI | 18 |
| KLKQDTTQK | 19 |
| LELFQHVSL | 20 |
| MAVNIVLQL | 21 |
| MLWKLVENV | 22 |
| QAWGAGLAY | 23 |
| SEADLQAEV | 24 |
| SLPDNVFSNL | 25 |
| SQGQIFSKF | 26 |
| SRLELFQHV | 27 |
| SSQGQIFSK | 28 |
| SVKERESER | 29 |
| VEEIFTEEY | 30 |
| VHDPDYAKILL | 31 |
| VPWEDKISI | 32 |
| VTTLALTF | 33 |
| WPMHTPLL | 34 |
| WPMHTPLLL | 35 |

or a nucleic acid encoding said TAP.
2. The TAP or nucleic acid of item 1, wherein the TAP binds to an HLA-A*02:01 molecule and comprises or consists of the sequence of SEQ ID NO: 22.
3. The TAP or nucleic acid of item 1, wherein the TAP binds to an HLA-A*03:01 molecule and comprises or consists of the sequence of SEQ ID NO: 19.
4. The TAP or nucleic acid of item 1, wherein the TAP binds to an HLA-A*11:01 molecule and comprises or consists of the sequence of SEQ ID NO: 1, 17 or 28.
5. The TAP or nucleic acid of item 1, wherein the TAP binds to an HLA-A*24:02 molecule and comprises or consists of the sequence of SEQ ID NO: 6 or 30.
6. The TAP or nucleic acid of item 1, wherein the TAP binds to an HLA-A*25:01 molecule and comprises or consists of the sequence of SEQ ID NO: 10.
7. The TAP or nucleic acid of item 1, wherein the TAP binds to an HLA-A*26:01 molecule and comprises or consists of the sequence of SEQ ID NO: 15.
8. The TAP or nucleic acid of item 1, wherein the TAP binds to an HLA-A*31:01 molecule and comprises or consists of the sequence of SEQ ID NO: 8, 9 or 29.
9. The TAP or nucleic acid of item 1, wherein the TAP binds to an HLA-A*33:01 molecule and comprises or consists of the sequence of SEQ ID NO: 2 or 3.
10. The TAP or nucleic acid of item 1, wherein the TAP binds to an HLA-B*15:01 molecule and comprises or consists of the sequence of SEQ ID NO: 26.
11. The TAP or nucleic acid of item 1, wherein the TAP binds to an HLA-B*18:01 molecule and comprises or consists of the sequence of SEQ ID NO: 13, 14 or 33.
12. The TAP or nucleic acid of item 1, wherein the TAP binds to an HLA-B*27:05 molecule and comprises or consists of the sequence of SEQ ID NO: 27.
13. The TAP or nucleic acid of item 1, wherein the TAP binds to an HLA-B*35:01 molecule and comprises or consists of the sequence of SEQ ID NO: 4, 12 or 23.
14. The TAP or nucleic acid of item 1, wherein the TAP binds to an HLA-B*35:03 molecule and comprises or consists of the sequence of SEQ ID NO: 38.
15. The TAP or nucleic acid of item 1, wherein the TAP binds to an HLA-B*38:01 molecule and comprises or consists of the sequence of SEQ ID NO: 34.
16. The TAP or nucleic acid of item 1, wherein the TAP binds to an HLA-B*40:01 molecule and comprises or consists of the sequence of SEQ ID NO: 20.
17. The TAP or nucleic acid of item 1, wherein the TAP binds to an HLA-B*49:01 molecule and comprises or consists of the sequence of SEQ ID NO: 11 or 24.
18. The TAP or nucleic acid of item 1, wherein the TAP binds to an HLA-B*50:01 molecule and comprises or consists of the sequence of SEQ ID NO: 5 or 7.
19. The TAP or nucleic acid of item 1, wherein the TAP binds to an HLA-B*51:01 molecule and comprises or consists of the sequence of SEQ ID NO: 35 or 37.
20. The TAP or nucleic acid of item 1, wherein the TAP binds to an HLA-B*52:01 molecule and comprises or consists of the sequence of SEQ ID NO: 18.
21. The TAP or nucleic acid of item 1, wherein the TAP binds to an HLA-B*58:01 molecule and comprises or consists of the sequence of SEQ ID NO: 36.
22. The TAP or nucleic acid of item 1, wherein the TAP binds to an HLA-C*01:02 molecule and comprises or consists of the sequence of SEQ ID NO: 25.
23. The TAP or nucleic acid of item 1, wherein the TAP binds to an HLA-C*12:03 molecule and comprises or consists of the sequence of SEQ ID NO: 16 or 21.
24. The TAP or nucleic acid of any one of items 1-23, which is encoded by a sequence located a non-protein coding region of the genome.
25. The TAP or nucleic acid of item 24, wherein said non-protein coding region of the genome is an intergenic region.
26. The TAP or nucleic acid of item 24, wherein said non-protein coding region of the genome is a long non-coding RNAs.
27. A combination comprising at least two of the TAPs or nucleic acids defined in any one of items 1-26.
28. The TAP or nucleic acid of any one of items 1 to 26, or the combination of item 27, wherein the nucleic acid is an mRNA.
29. The TAP or nucleic acid of any one of items 1 to 26, or the combination of item 27, wherein the nucleic acid is a DNA.
30. The TAP or nucleic acid of any one of items 1 to 26, or the combination of item 27, wherein the nucleic acid is a component of a viral vector.
31. A synthetic long peptide (SLP) comprising at least one of the amino acid sequences defined in item 1, or a nucleic acid encoding the SLP.
32. The SLP or nucleic acid of item 31, wherein the SLP comprises at least 5, 10, 15 or 20 of the amino acid sequences defined in claim 1.
33. A vesicle or particle comprising the TAP, nucleic acid, combination or SLP of any one of items 1 to 32.
34. The vesicle or particle of item 33, wherein the vesicle is a lipid nanoparticle (LNP).
35. The vesicle or particle of item 33 or 34, which comprises a cationic lipid.
36. A composition comprising the TAP, nucleic acid, combination or SLP of any one of items 1 to 32, or the vesicle or particle of any one of items 33-35, and a pharmaceutically acceptable carrier.
37. A vaccine comprising the TAP, nucleic acid, combination or SLP of any one of items 1 to 32, the vesicle or particle of any one of items 33-35, or the composition of item 36, and an adjuvant.
38. An isolated major histocompatibility complex (MHC) class I molecule comprising the TAP of any one of items 1-26 in its peptide binding groove.
39. The isolated MHC class I molecule of item 38, which is in the form of a multimer.
40. The isolated MHC class I molecule of item 39, wherein said multimer is a tetramer.
41. An isolated cell comprising (i) the TAP of any one of items 1-26, (ii) the combination of item 27; (iii) the SLP of item 31 or 32; or (iv) a vector comprising a nucleotide sequence encoding the TAP of any one of items 1-26, the combination of item 27 or the SLP of item 31 or 32.
42. An isolated cell expressing at its surface major histocompatibility complex (MHC) class I molecules comprising the TAP or combination of any one of items 1-30 in their peptide binding groove.
43. The cell of item 41 or 42, which is an antigen-presenting cell (APC).
44. The cell of item 43, wherein said APC is a dendritic cell.
45. A T-cell receptor (TCR) that specifically recognizes the isolated MHC class I molecule of any one of items 38-40 and/or MHC class I molecules expressed at the surface of the cell of any one of items 42-44.
46. The TCR of item 45, which is a soluble TCR.
47. An antibody or an antigen-binding fragment thereof that specifically binds to the isolated MHC class I molecule of any one of items 38-40 and/or MHC class I molecules expressed at the surface of the cell of any one of items 42-44.
48. The TCR of item 45 or 46, or the antibody or antigen-binding fragment thereof according to item 47, which is a bispecific TCR or a bispecific antibody or antigen-binding fragment thereof.
49. The TCR, antibody or antigen-binding fragment thereof according to item 48, wherein the bispecific antibody or antigen-binding fragment thereof is a single-chain diabody (scDb).
50. The TCR, antibody or antigen-binding fragment thereof according to item 48 or 49, wherein the bispecific TCR, antibody or antigen-binding fragment thereof also specifically binds to a T cell signaling molecule.
51. The TCR, antibody or antigen-binding fragment thereof according to item 50, wherein the T cell signaling molecule is a CD3 chain.
52. A chimeric antigen receptor (CAR) comprising the antibody or an antigen-binding fragment thereof of item 47, or a nucleic acid encoding said CAR.
53. An isolated cell expressing at its cell surface the TCR of item 45.
54. The isolated cell of item 53, which is a CD8+ T lymphocyte.
55. A cell population comprising at least 0.5% or 1% of the isolated cell as defined in item 53 or 54.
56. A method of treating breast cancer in a subject comprising administering to the subject an effective amount of:
(a) a TAP comprising or consisting of any one of the sequences set forth in SEQ ID NOs: 1-61 or any combination thereof, or a synthetic long peptide (SLP) comprising at least one of the sequences set forth in SEQ ID NOs: 1-61;
(b) at least one nucleic acid encoding the TAP, combination thereof or SLP defined in (a);
(c) a vesicle or particle comprising the TAP, combination thereof or SLP defined in (a) or the at least one nucleic acid defined in (b);
(d) a composition comprising the TAP, combination thereof or SLP defined in (a), the at least one nucleic acid defined in (b), or the vesicle or particle defined in (c), and a pharmaceutically acceptable carrier;
(e) a vaccine comprising the TAP, combination thereof or SLP defined in (a), the at least one nucleic acid defined in (b), the vesicle or particle defined in (c), or the composition defined in (d), and an adjuvant;
(f) a cell expressing at its surface major histocompatibility complex (MHC) class I molecules comprising the TAP or combination thereof defined in (a) in their peptide binding groove;
(g) a cell expressing at its cell surface a T-cell receptor (TCR) that specifically recognizes MHC class I molecules expressed at the surface of the cell defined in (f); or
(h) a soluble TCR, an antibody or an antigen-binding fragment thereof that specifically binds to the MHC class I molecules expressed at the surface of the cell defined in (f).
57. The method of item 56, wherein the breast cancer is hormone-receptor-positive breast cancer (HR+) or triple-negative breast cancer (TNBC).
58. The method of item 56 or 57, further comprising administering at least one additional antitumor agent or therapy to the subject.
59. The method of item 58, wherein said at least one additional antitumor agent or therapy is a chemotherapeutic agent, immunotherapy, an immune checkpoint inhibitor, radiotherapy or surgery.
60. Use of:
(a) a TAP comprising or consisting of any one of the sequences set forth in SEQ ID NOs: 1-61 or any combination thereof, or a synthetic long peptide (SLP) comprising at least one of the sequences set forth in SEQ ID NOs: 1-61;
(b) at least one nucleic acid encoding the TAP, combination thereof or SLP defined in (a);
(c) a vesicle or particle comprising the TAP, combination thereof or SLP defined in (a) or the at least one nucleic acid defined in (b);
(d) a composition comprising the TAP, combination thereof or SLP defined in (a), the at least one nucleic acid defined in (b), or the vesicle or particle defined in (c), and a pharmaceutically acceptable carrier;
(e) a vaccine comprising the TAP, combination thereof or SLP defined in (a), the at least one nucleic acid defined in (b), the vesicle or particle defined in (c), or the composition defined in (d), and an adjuvant;
(f) a cell expressing at its surface major histocompatibility complex (MHC) class I molecules comprising the TAP or combination thereof defined in (a) in their peptide binding groove;
(g) a cell expressing at its cell surface a T-cell receptor (TCR) that specifically recognizes MHC class I molecules expressed at the surface of the cell defined in (f); or
(h) a soluble TCR, an antibody or an antigen-binding fragment thereof that specifically binds to the MHC class I molecules expressed at the surface of the cell defined in (f);
for treating breast cancer in a subject, or for the manufacture of a medicament for treating breast cancer in a subject.
61. The use of item 60, wherein the breast cancer is hormone-receptor-positive breast cancer (HR+) or triple-negative breast cancer (TNBC).
62. The use of item 60 or 61, further comprising the use at least one additional antitumor agent or therapy to the subject.
63. The use of item 62, wherein said at least one additional antitumor agent or therapy is a chemotherapeutic agent, immunotherapy, an immune checkpoint inhibitor, radiotherapy or surgery.
64. An agent for use in treating breast cancer in a subject, wherein the agent is:
(a) a TAP comprising or consisting of any one of the sequences set forth in SEQ ID NOs: 1-61 or any combination thereof, or a synthetic long peptide (SLP) comprising at least one of the sequences set forth in SEQ ID NOs: 1-61;
(b) at least one nucleic acid encoding the TAP, combination thereof or SLP defined in (a);
(c) a vesicle or particle comprising the TAP, combination thereof or SLP defined in (a) or the at least one nucleic acid defined in (b);
(d) a composition comprising the TAP, combination thereof or SLP defined in (a), the at least one nucleic acid defined in (b), or the vesicle or particle defined in (c), and a pharmaceutically acceptable carrier;
(e) a vaccine comprising the TAP, combination thereof or SLP defined in (a), the at least one nucleic acid defined in (b), the vesicle or particle defined in (c), or the composition defined in (d), and an adjuvant;
(f) a cell expressing at its surface major histocompatibility complex (MHC) class I molecules comprising the TAP or combination thereof defined in (a) in their peptide binding groove;
(g) a cell expressing at its cell surface a T-cell receptor (TCR) that specifically recognizes MHC class I molecules expressed at the surface of the cell defined in (f); or
(h) a soluble TCR, an antibody or an antigen-binding fragment thereof that specifically binds to the MHC class I molecules expressed at the surface of the cell defined in (f).
65. The agent for use of item 64, wherein the breast cancer is hormone-receptor-positive breast cancer (HR+) or triple-negative breast cancer (TNBC).
66. The agent for use of item 64 or 65, further comprising the use at least one additional antitumor agent or therapy to the subject.
67. The agent for use of item 66leisftqffi, wherein said at least one additional antitumor agent or therapy is a chemotherapeutic agent, immunotherapy, an immune checkpoint inhibitor, radiotherapy or surgery.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A-E depict the canonical immunopeptidomes of HR⁺ and TNBC tumors. FIG. 1A: Number of unique MAPs identified per sample. FIG. 1B: Venn diagram of MAPs source genes in HR+ and TNBC breast cancer tumors and normal tissues from the HLA ligand ATLAS. FIG. 1C: Total number of MAPs per source gene for the top 1% generators of MAPs. FIG. 1D: PANTHER analysis for the top 1% generators of MAPs (n=242) show an enrichment in cytoskeleton, transcription and extracellular matrix protein classes. FIG. 1E: Expression of genes non-sources of MAPs, sources of MAPs and top 1% generators of MAPs (ANOVA; p<0.001).

FIGS. 2A-D show that ERE-derived MAPs populate the immunopeptidome of HR⁺ and TNBC tumors. FIG. 2A: Similar number of ERE-derived MAPs are identified in HR⁺ (n=14) and TNBC samples (n=12). FIG. 2B: Number of ERE-derived MAPs per ERE class. FIG. 2C: Number of MAPs identified per ERE family in HR⁺ and TNBC samples. FIG. 2D: ERE families that lead to MAP generation have a higher expression at the transcriptomic level than non source families in tumors from the TCGA cohort (t-test; p<0.001, n=741).

FIGS. 3A-C show the identification of tumor antigens of interest. FIG. 3A: Classification workflow for MAPs of interest. FIG. 3B: Number of TSAs and TAAs identified in HR⁺ samples, TNBC samples or both. Most TSAs were identified solely in TNBC samples (n=18/25). FIG. 3C: Most of the TSAs and TAAs identified with the classification workflow are novel.

FIGS. 4A-D show the identification of TSAs. FIG. 4A: Expression heatmap of TSAs' coding sequence in normal tissues (GTEX, mTEC and bone marrow). FIG. 4B: Genomic origin of TSAs. Identified aeTSAs mainly originate from exonic regions. FIG. 4C: Percentage of HR⁺ and TNBC tumors with individual TAA expression >2 rphm. FIG. 4D: GSEA analysis of the TCGA breast cancer cohort shows an enrichment of tumor infiltrating leucocytes gene markers in tumors with a high level (>median) of predicted TSAs.

FIGS. 5A-C show the identification of TAAs. FIG. 5A: Expression heatmap of TAAs' coding sequence in normal tissues (GTEX, mTEC and bone marrow) regrouped by source genes' associated function. FIG. 5B: Percentage of HR⁺ and TNBC tumors with individual TAA expression >2 rphm. FIG. 5C: GSEA analysis in the HR⁺ breast cancer TCGA cohort shows an enrichment in both immunosuppressive pathways and immune activation pathways in tumors with a high level (>median) of predicted TAAs (as defined by: expression >2 rphm and adequate HLA allele for presentation).

FIG. 6 show CAFs-derived TAAs. Expression of COL11A1, COL10A1 and LRRC15 in different cells in from the microenvironment of breast cancer samples.

FIGS. 7A-E show survival analysis of predicted TSAs and TSAs in the TCGA cohort. FIG. 7A: Survival analysis of highly expressed predTSAs in HR+ breast cancer tumors show no survival impact. FIGS. 7B-C: Survival analysis of highly expressed predTAAs in TNBC and HR+ breast cancer tumors show no survival impact. FIGS. 7D-E: Survival analysis in the TNBC cohort of TCGA shows a significative survival benefit in patients with a high level of highly expressed predTSAs originating from non-coding regions and CTAs, whereas high expression of TSAs from non-coding regions or CTAs without potential presentation is associated with a reduced survival.

FIG. 8 is a schematic of the database construction for MAP identification include a canonical proteome, an ERE proteome, a small-RNA proteome (smRNA) and a cancer specific proteome.

FIG. 9 shows the number of MAPs identified for each HLA allele (n=53) in the dataset (n=26).

FIG. 10A shows the percentage of the transcriptome represented at the immunopeptidomic level per cumulative samples.

FIG. 10B is a histogram of the total number of MAPs generated per source gene in the dataset.

FIGS. 11A-B is a PANTHER enrichment analysis of source genes exclusive to cancer immunopeptidomes. FIG. 11A: Source genes shared between HR⁺ and TNBC samples (n=277) FIG. 11B: Source genes exclusive to TNBC tumors (n=259).

FIG. 12A is a schematic of the small-RNA database construction workflow. FIG. 12B is a schematic of the filtering steps for peptide annotation and validation.

FIG. 13 is a genomic mapping of identified ERE-derived MAPs.

FIG. 14 is a Spearman correlation of the frequency of mutations per gene in the breast cancer TCGA cohort and the total number of MAPs identified per gene in the dataset.

FIG. 15 shows a GSEA analysis of breast cancer tumors from TCGA (n=1109). The GSEA analysis shows an enrichment in mitotic and immune activation pathways in tumors with a high level (>median) of predicted TSAs (as defined by: expression >2 rphm and adequate HLA allele for presentation). An enrichment in the PI3K signaling genes can be observed in tumors with a low level of predicted TSAs.

FIG. 16 shows a GSEA analysis in the TNBC cohort of TCGA. The GSEA analysis shows an enrichment in immune activation and immunosuppressive pathways in tumors with a high level (>median) of predicted TAAs (as defined by: expression >2 rphm and adequate HLA allele for presentation).

FIG. 17 shows a survival analysis in the TNBC cohort of TCGA showing a significative survival benefit in patients with a high level of highly expressed predTSAs.

FIGS. 18A and 18B depict the results of Functional Expansion of Specific T cells (FEST) assays showing significant antigen-specific clonotype expansion for selected TSAs and TAAs in donors D26 (FIG. 18A) and D27 (FIG. 18B) after 20 days stimulation with autologous T cell-depleted PBMCs pulsed with individual peptides. For each peptide: left circles=CD8 T cells cultured with peptide; middle circles=CD8 T cells cultured without peptide; right circles: =uncultured CD8 T cells. Control peptide=MelanA, ELAGIGILTV (SEQ ID NO:212)

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein, the present inventors have identified TSA and TAA candidates from breast cancer specimens using a proteogenomic-based approach. The novel TSA and TAA candidates identified herein may be useful, e.g., for immunotherapies and vaccines against cancers expressing the TSA and TAA candidates, such as breast cancer.

The present disclosure relates to a tumor antigen peptide (TAP) (or tumor-specific peptide), such as a breast cancer (BC) TAP, comprising, or consisting of, one of the following amino acid sequences:

| Peptide sequence | SEQ ID NO: |
|---|---|
| SVLGPILIIPK | 1 |
| DMFGIRFKR | 2 |
| EYNAEFGRR | 3 |
| FPEDFSILF | 4 |
| GEAVGFQPP | 5 |
| IYLIPEPVLF | 6 |
| LEISFISPL | 7 |
| RSMRVGVTR | 8 |
| SMRSTVVSR | 9 |
| SVADLLSSF | 10 |
| AETDITETSI | 11 |
| DIIEYDYEY | 12 |
| EEIFTEEY | 13 |
| EEQYIAQF | 14 |
| ELFQHVSLM | 15 |
| GAYDKARYL | 16 |
| GTHVWVGLYK | 17 |
| HQFTEKVI | 18 |
| KLKQDTTQK | 19 |
| LELFQHVSL | 20 |
| MAVNIVLQL | 21 |
| MLWKLVENV | 22 |
| QAWGAGLAY | 23 |
| SEADLQAEV | 24 |
| SLPDNVFSNL | 25 |
| SQGQIFSKF | 26 |
| SRLELFQHV | 27 |
| SSQGQIFSK | 28 |
| SVKERESER | 29 |
| VEEIFTEEY | 30 |
| VHDPDYAKILL | 31 |
| VPWEDKISI | 32 |
| VTTLALTF | 33 |
| WPMHTPLL | 34 |
| WPMHTPLLL | 35 |

In another aspect, the present disclosure further relates to the use of a TAP comprising, or consisting of, one of the amino acid sequences below, for the treatment of breast cancer:

| Peptide sequence | SEQ ID NO: |
|---|---|
| SVLGPILIIPK | 1 |
| DMFGIRFKR | 2 |
| EYNAEFGRR | 3 |
| FPEDFSILF | 4 |
| GEAVGFQPP | 5 |
| IYLIPEPVLF | 6 |
| LEISFISPL | 7 |
| RSMRVGVTR | 8 |
| SMRSTVVSR | 9 |
| SVADLLSSF | 10 |
| AETDITETSI | 11 |
| DIIEYDYEY | 12 |
| EEIFTEEY | 13 |
| EEQYIAQF | 14 |
| ELFQHVSLM | 15 |
| GAYDKARYL | 16 |
| GTHVWVGLYK | 17 |
| HQFTEKVI | 18 |
| KLKQDTTQK | 19 |
| LELFQHVSL | 20 |
| MAVNIVLQL | 21 |
| MLWKLVENV | 22 |
| QAWGAGLAY | 23 |
| SEADLQAEV | 24 |
| SLPDNVFSNL | 25 |
| SQGQIFSKF | 26 |
| SRLELFQHV | 27 |
| SSQGQIFSK | 28 |
| SVKERESER | 29 |
| VEEIFTEEY | 30 |
| VHDPDYAKILL | 31 |
| VPWEDKISI | 32 |
| VTTLALTF | 33 |
| WPMHTPLL | 34 |
| WPMHTPLLL | 35 |
| EADPTGHSY | 36 |
| EVDPASNTY | 37 |
| EVDPTSHSY | 38 |
| EVYDGREHSAY | 39 |
| FVQENYLEY | 40 |
| GEALGLVGA | 41 |
| LVQEKYLEY | 42 |
| TFLQRKVLR | 43 |
| DETLTALQF | 44 |
| EVFEGDIQQF | 45 |
| EVKSITKER | 46 |
| FAFEKLIQF | 47 |
| HHNDLVFKF | 48 |
| ITDDQLLAL | 49 |
| LEIENLNLA | 50 |
| MPLKHYLLL | 51 |
| MVFSKLVLR | 52 |
| QLLALLPSL | 53 |
| TTLENLPQK | 54 |
| VEAPHLPSF | 55 |
| DELAHFLL | 56 |
| FPDLETSF | 57 |
| EELSVLEVF | 58 |
| EELSVMEVY | 59 |
| FPDLESEF | 60 |
| YEFLWGSKA | 61 |

In general, peptides such as tumor antigen peptides (TAPs) presented in the context of HLA class I vary in length from about 7 or 8 to about 15, or preferably 8 to 14 amino acid residues. In some embodiments of the methods of the disclosure, longer peptides comprising the TAP sequences defined herein are artificially loaded into cells such as antigen presenting cells (APCs), processed by the cells and the TAP is presented by MHC class I molecules at the surface of the APC. In this method, peptides/polypeptides longer than 15 amino acid residues can be loaded into APCs, are processed by proteases in the APC cytosol providing the corresponding TAP as defined herein for presentation. In some embodiments, the precursor peptide/polypeptide that is used to generate the TAP defined herein is for example 1000, 500, 400, 300, 200, 150, 100, 75, 50, 45, 40, 35, 30, 25, 20 or 15 amino acids or less. Thus, all the methods and processes using the TAPs described herein include the use of longer peptides or polypeptides (including the native protein), i.e., tumor antigen precursor peptides/polypeptides, to induce the presentation of the “final” 8-14 TAP following processing by the cell (APCs). In some embodiments, the herein-mentioned TAP is about 8 to 14, 8 to 13, or 8 to 12 amino acids long (e.g., 8, 9, 10, 11, 12 or 13 amino acids long), small enough for a direct fit in an HLA class I molecule. In an embodiment, the TAP comprises 20 amino acids or less, preferably 15 amino acids or less, more preferably 14 amino acids or less. In an embodiment, the TAP comprises at least 7 amino acids, preferably at least 8 amino acids or less, more preferably at least 9 amino acids.

The term “amino acid” as used herein includes both L- and D-isomers of the naturally occurring amino acids as well as other amino acids (e.g., naturally-occurring amino acids, non-naturally-occurring amino acids, amino acids which are not encoded by nucleic acid sequences, etc.) used in peptide chemistry to prepare synthetic analogs of TAPs. Examples of naturally occurring amino acids are glycine, alanine, valine, leucine, isoleucine, serine, threonine, etc. Other amino acids include for example non-genetically encoded forms of amino acids, amino acid analogs as well as a conservative substitution of an L-amino acid. Naturally-occurring non-genetically encoded amino acids and amino acid analogs include, for example, beta-alanine, 3-amino-propionic acid, 2,3-diaminopropionic acid, alpha-aminoisobutyric acid (Aib), 4-amino-butyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, norleucine (Nle), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (Hoarg), N-acetyl lysine, 2-amino butyric acid, 2-amino butyric acid, 2,4-diaminobutyric acid (D- or L-), p-aminophenylalanine, N-methylvaline, homocysteine, homoserine (HoSer), cysteic acid, epsilon-amino hexanoic acid, delta-amino valeric acid, benzyloxy-tyrosine, β-phenylalanine or 2,3-diaminobutyric acid (D- or L-), etc. These amino acids are well known in the art of biochemistry/peptide chemistry. Thus, one or more of the amino acids in the TAPs described herein (SEQ ID NOs:1-61 or SEQ ID NOs:1-35) may be replaced by a non-genetically encoded amino acid and/or an amino acid analog. The TAPs may also be modified to improve the proteolytic stability of the peptides, for example by the incorporation of methyl-amino acids, β-amino acids or peptoids. In an embodiment, the TAP comprises only naturally-occurring amino acids.

In embodiments, the TAPs described herein include peptides with altered sequences containing substitutions of functionally equivalent amino acid residues, relative to the herein-mentioned sequences. For example, one or more amino acid residues within the sequence can be substituted by another amino acid of a similar polarity (having similar physico-chemical properties) which acts as a functional equivalent, resulting in a silent alteration. Substitution for an amino acid within the sequence may be selected from other members of the class to which the amino acid belongs. For example, positively charged (basic) amino acids include arginine, lysine and histidine (as well as homoarginine and ornithine). Nonpolar (hydrophobic) amino acids include leucine, isoleucine, alanine, phenylalanine, valine, proline, tryptophan and methionine. Uncharged polar amino acids include serine, threonine, cysteine, tyrosine, asparagine and glutamine. Negatively charged (acidic) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. The herein-mentioned TAP may comprise all L-amino acids, all D-amino acids or a mixture of L- and D-amino acids. In an embodiment, the herein-mentioned BC TAP comprises all L-amino acids.

In an embodiment, in the sequences of the TAPs comprising or consisting of one of sequences of SEQ ID NOs:1-61 or SEQ ID NOs:1-35, the amino acid residues that do not substantially contribute to interactions with the T-cell receptor may be modified by replacement with other amino acid whose incorporation does not substantially affect T-cell reactivity and does not eliminate binding to the relevant MHC.

The TAP may also be modified by replacing one or more of the amide bonds of the peptide that may improve chemical stability and/or enhanced biological/pharmacological properties (e.g., half-life, absorption, potency, efficiency, etc.). Typical peptide bond replacements include esters, polyamines and derivatives thereof as well as substituted alkanes and alkenes, such as aminomethyl and ketomethylene. For example, the above-mentioned TAP may have one or more amide bonds replaced by linkages such as —CH₂NH—, —CH₂S—, —CH₂—CH₂—, —CH═CH— (cis or trans), —CH₂SO—, —CH(OH)CH₂—, or —COCH₂—.

The TAP may also be N- and/or C-terminally capped or modified to prevent degradation, increase stability, affinity and/or uptake. Thus, in another aspect, the present disclosure provides a modified TAP of the formula Z¹-X-Z², wherein X is a TAP comprising, or consisting of, one of the amino acid sequences of SEQ ID NOs:1-61 or SEQ ID NOs:1-35.

In an embodiment, the amino terminal residue (i.e., the free amino group at the N-terminal end) of the TAP is modified (e.g., for protection against degradation), for example by covalent attachment of a moiety/chemical group (Z¹). Z¹ may be a straight chained or branched alkyl group of one to eight carbons, or an acyl group (R—CO—), wherein R is a hydrophobic moiety (e.g., acetyl, propionyl, butanyl, iso-propionyl, or iso-butanyl), or an aroyl group (Ar—CO—), wherein Ar is an aryl group. In an embodiment, the acyl group is a C₁-C₁₆ or C₃-C₁₆ acyl group (linear or branched, saturated or unsaturated), in a further embodiment, a saturated C₁-C₆ acyl group (linear or branched) or an unsaturated C₃-C₆ acyl group (linear or branched), for example an acetyl group (CH₃—CO—, Ac). In an embodiment, Z¹ is absent. The carboxy terminal residue (i.e., the free carboxy group at the C-terminal end of the TAP) of the TAP may be modified (e.g., for protection against degradation), for example by amidation (replacement of the OH group by a NH₂ group), thus in such a case Z² is a NH₂ group. In an embodiment, Z² may be an hydroxamate group, a nitrile group, an amide (primary, secondary or tertiary) group, an aliphatic amine of one to ten carbons such as methyl amine, iso-butylamine, iso-valerylamine or cyclohexylamine, an aromatic or arylalkyl amine such as aniline, napthylamine, benzylamine, cinnamylamine, or phenylethylamine, an alcohol or CH₂OH. In an embodiment, Z² is absent. In an embodiment, the TAP comprises one of the amino acid sequences of SEQ ID NOs:1-61 or SEQ ID NOs:1-35. In an embodiment, the TAP consists of one of the amino acid sequences of SEQ ID NOs:1-61 or SEQ ID NOs:1-35, i.e., wherein Z¹ and Z² are absent.

In another aspect, the present disclosure provides a TAP binding to an HLA-A*02:01 molecule, comprising or consisting of the sequence of SEQ ID NO: 22. Because HLA alleles show promiscuity (certain HLA alleles present similar epitopes), the above-identified TAP may further bind to HLA-A*02:05, HLA-A*02:06 and/or HLA-A*02:07 molecules.

In another aspect, the present disclosure provides a TAP binding to an HLA-A*03:01 molecule, comprising or consisting of the sequence of SEQ ID NO: 19. Because HLA alleles show promiscuity (certain HLA alleles present similar epitopes), the above-identified TAP may further bind to HLA-A*03:02 or HLA-A*30:01 molecules.

In another aspect, the present disclosure provides a TAP binding to an HLA-A*11:01 molecule, comprising or consisting of the sequence of SEQ ID NO: 10, 17 or 28. Because HLA alleles show promiscuity (certain HLA alleles present similar epitopes), the above-identified TAP may further bind to HLA-A*03:01, HLA-A*31:01 and/or HLA-A*68:01 molecules.

In another aspect, the present disclosure provides a TAP binding to an HLA-A*24:02 molecule, comprising or consisting of the sequence of SEQ ID NO:6. Because HLA alleles show promiscuity (certain HLA alleles present similar epitopes), the above-identified TAP may further bind to HLA-A*23:01 molecules.

In another aspect, the present disclosure provides a TAP binding to an HLA-A*25:01 molecule, comprising or consisting of the sequence of SEQ ID NO: 1. Because HLA alleles show promiscuity (certain HLA alleles present similar epitopes), the above-identified TAP may further bind HLA-A*26:01, HLA-A*66:01 or HLA-B*15:02 molecules.

In another aspect, the present disclosure provides a TAP binding to an HLA-A*26:01 molecule, comprising or consisting of the sequence of SEQ ID NO: 15. Because HLA alleles show promiscuity (certain HLA alleles present similar epitopes), the above-identified TAP may further bind to HLA-A*25:01 or HLA-A*66:01 molecules.

In another aspect, the present disclosure provides a TAP binding to an HLA-A*31:01 molecule, comprising or consisting of the sequence of SEQ ID NO: 8, 9 or 29. Because HLA alleles show promiscuity (certain HLA alleles present similar epitopes), the above-identified TAP may further bind to HLA-A*11:01, HLA-A*33:01, HLA-A*33:03 or HLA-A*68:01 molecules.

In another aspect, the present disclosure provides a TAP binding to an HLA-A*33:01 molecule, comprising or consisting of the sequence of SEQ ID NO: 2 or 3. Because HLA alleles show promiscuity (certain HLA alleles present similar epitopes), the above-identified TAP may further bind to HLA-A*31:01, HLA-A*33:03 or HLA-A*68:01 molecules.

In another aspect, the present disclosure provides a TAP binding to an HLA-B*15:01 molecule, comprising or consisting of the sequence of SEQ ID NO: 26. Because HLA alleles show promiscuity (certain HLA alleles present similar epitopes), the above-identified TAP may further bind to HLA-A*25:01, HLA-A*29:02, HLA-B*15:01, HLA-B*15:03, HLA-B*15:18, HLA-B*35:01 or HLA-B*46:01 molecules.

In another aspect, the present disclosure provides a TAP binding to an HLA-B*18:01 molecule, comprising or consisting of the sequence of SEQ ID NO: 13, 14 or 30. Because HLA alleles show promiscuity (certain HLA alleles present similar epitopes), the above-identified TAP may further bind to HLA-B*40:01, HLA-B*44:02, HLA-B*44:03 and/or HLA-B*45:01 molecules.

In another aspect, the present disclosure provides a TAP binding to an HLA-B*27:05 molecule, comprising or consisting of the sequence of SEQ ID NO: 27. Because HLA alleles show promiscuity (certain HLA alleles present similar epitopes), the above-identified TAP may further bind to HLA-B*27:02.

In another aspect, the present disclosure provides a TAP binding to an HLA-B*35:01 molecule, comprising or consisting of the sequence of SEQ ID NO: 4, 12, 23. Because HLA alleles show promiscuity (certain HLA alleles present similar epitopes), the above-identified TAP may further bind to HLA-B*15:02, HLA-B*35:02, HLA-B*35:03 or HLA-B*53:01 molecules.

In another aspect, the present disclosure provides a TAP binding to an HLA-B*35:03 molecule, comprising or consisting of the sequence of SEQ ID NO: 35. Because HLA alleles show promiscuity (certain HLA alleles present similar epitopes), the above-identified TAP may further bind to HLA-B*07:02, HLA-B*35:01, HLA-B*35:03, HLA-B*51:01, HLA-B*53:01, HLA-B*55:01 or HLA-B*56:01 molecules.

In another aspect, the present disclosure provides a TAP binding to an HLA-B*35:08 molecule, comprising or consisting of the sequence of SEQ ID NO: 35.

In another aspect, the present disclosure provides a TAP binding to an HLA-B*38:01 molecule, comprising or consisting of the sequence of SEQ ID NO: 31. Because HLA alleles show promiscuity (certain HLA alleles present similar epitopes), the above-identified TAP may further bind to HLA-B*39:01 molecules.

In another aspect, the present disclosure provides a TAP binding to an HLA-B*40:01 molecule, comprising or consisting of the sequence of SEQ ID NO: 20. Because HLA alleles show promiscuity (certain HLA alleles present similar epitopes), the above-identified TAP may further bind to HLA-molecules.

In another aspect, the present disclosure provides a TAP binding to an HLA-B*49:01 molecule, comprising or consisting of the sequence of SEQ ID NO: 11 or 24. Because HLA alleles show promiscuity (certain HLA alleles present similar epitopes), the above-identified TAP may further bind to HLA-B*18:01, HLA-B*40:02, HLA-B*41:02, HLA-B*44:02, HLA-B*44:03 or HLA-B*45:01 molecules.

In another aspect, the present disclosure provides a TAP binding to an HLA-B*50:01 molecule, comprising or consisting of the sequence of SEQ ID NO: 5 or 7.

In another aspect, the present disclosure provides a TAP binding to an HLA-B*51:01 molecule, comprising or consisting of the sequence of SEQ ID NO: 32 or 34. Because HLA alleles show promiscuity (certain HLA alleles present similar epitopes), the above-identified TAP may further bind to HLA-B*52:01 molecules.

In another aspect, the present disclosure provides a TAP binding to an HLA-B*52:01 molecule, comprising or consisting of the sequence of SEQ ID NO: 18. Because HLA alleles show promiscuity (certain HLA alleles present similar epitopes), the above-identified TAP may further bind to HLA-B*51:01 molecules.

In another aspect, the present disclosure provides a TAP binding to an HLA-B*58:01 molecule, comprising or consisting of the sequence of SEQ ID NO: 33. Because HLA alleles show promiscuity (certain HLA alleles present similar epitopes), the above-identified TAP may further bind to HLA-A*32:01 or HLA-B*57:01 molecules.

In another aspect, the present disclosure provides a TAP binding to an HLA-C*01:02 molecule, comprising or consisting of the sequence of SEQ ID NO: 25.

In another aspect, the present disclosure provides a TAP binding to an HLA-C*12:03 molecule, comprising or consisting of the sequence of SEQ ID NO: 16 or 21. Because HLA alleles show promiscuity (certain HLA alleles present similar epitopes), the above-identified TAP may further bind to HLA-B*46:01, HLA-C*03:02, HLA-C*03:03, HLA-C*03:04, HLA-C*08:01, HLA-C*12:03, HLA-C*15:02 or HLA-C*16:01 molecules.

The TAPs of the disclosure may be produced by expression in a host cell comprising a nucleic acid encoding the TAPs (recombinant expression) or by chemical synthesis (e.g., solid-phase peptide synthesis). Peptides can be readily synthesized by manual and/or automated solid phase procedures well known in the art. Suitable syntheses can be performed for example by utilizing “T-boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the TAPs may be prepared by way of segment condensation, as described, for example, in Liu et al., Tetrahedron Lett. 37: 933-936, 1996; Baca et al., J. Am. Chem. Soc. 117: 1881-1887, 1995; Tam et al., Int. J. Peptide Protein Res. 45: 209-216, 1995; Schnolzer and Kent, Science 256: 221-225, 1992; Liu and Tam, J. Am. Chem. Soc. 116: 4149-4153, 1994; Liu and Tam, Proc. Natl. Acad. Sci. USA 91: 6584-6588, 1994; and Yamashiro and Li, Int. J. Peptide Protein Res. 31: 322-334, 1988). Other methods useful for synthesizing the TAPs are described in Nakagawa et al., J. Am. Chem. Soc. 107: 7087-7092, 1985. In an embodiment, the TAP is chemically synthesized (synthetic peptide). Another embodiment of the present disclosure relates to a non-naturally occurring peptide wherein said peptide consists or consists essentially of an amino acid sequences defined herein and has been synthetically produced (e.g., synthesized) as a pharmaceutically acceptable salt. The salts of the TAPs according to the present disclosure differ substantially from the peptides in their state(s) in vivo, as the peptides as generated in vivo are no salts. The non-natural salt form of the peptide may modulate the solubility of the peptide, in particular in the context of pharmaceutical compositions comprising the peptides, e.g., the peptide vaccines as disclosed herein. Preferably, the salts are pharmaceutically acceptable salts of the peptides.

In an embodiment, the herein-mentioned TAP is substantially pure. A compound is “substantially pure” when it is separated from the components that naturally accompany it. Typically, a compound is substantially pure when it is at least 60%, more generally 75%, 80% or 85%, preferably over 90% and more preferably over 95%, by weight, of the total material in a sample. Thus, for example, a polypeptide that is chemically synthesized or produced by recombinant technology will generally be substantially free from its naturally associated components, e.g., components of its source macromolecule. A nucleic acid molecule is substantially pure when it is not immediately contiguous with (i.e., covalently linked to) the coding sequences with which it is normally contiguous in the naturally occurring genome of the organism from which the nucleic acid is derived. A substantially pure compound can be obtained, for example, by extraction from a natural source; by expression of a recombinant nucleic acid molecule encoding a peptide compound; or by chemical synthesis. Purity can be measured using any appropriate method such as column chromatography, gel electrophoresis, HPLC, etc. In an embodiment, the TAP is in solution. In another embodiment, the TAP is in solid form, e.g., lyophilized.

In an embodiment, the TAP is encoded by a sequence located a non-protein coding region of the genome. In an embodiment, the TAP is encoded by a sequence located in an intergenic region. In another embodiment, the TAP is encoded by a non-coding RNA (ncRNA).

In another aspect, the disclosure further provides a synthetic long peptide (SLP) comprising at least one of the TAP described herein. In an embodiment, the SLP comprises at least two TAPs, wherein at least one of the TAP is a TAP as described herein. In an embodiment, the SLP comprises at least 2, 3, 4, 5, 10, 15, 20, 25, 30, 35 or 40 of the TAPs described herein. In an embodiment, the SLP comprises at least one of the TAPs described herein linked to one or more amino acid sequences or domains that confer desired properties to the SLP, such as sequences or domains that stabilize the SLP and/or that improve processing and presentation by MHC molecules, for example a sequence comprising a motif cleavable by cellular proteases such as cathepsins. In another embodiment, the SLP comprises at least one of the TAPs described herein, and a TAP that binds to MHC class II molecules. The TAPs may directly attached to each other, or may be indirectly attached via a linker such as a short amino acid linker. In embodiments, the linker comprises about 4 to about 20 amino acids, or about 4 to about 15 amino acids, e.g., 4, 5, 6, 7, 8, 9, 10, 11, 12, 13, 14 or 15 amino acids. In an embodiment, the linker comprises glycine residues, serine residues, proline residues, threonine residues, or a mixture thereof. The linker may include sequences promoting the processing of the SLP to release the TAPs, such as a cathepsin-sensitive linker (e.g., a linker of 4-6 amino acids comprising the sequence LVGS, ASLG, PIVG, LLSV, VLSVG or LLSVGG, see Rabu et al., Oncoimmunology. 2019; 8(4): e1560919). In an embodiment, the SLP has a length of 500, 400, 300, 200, 150, 100, 90, 80, 70, 60 or 50 amino acids or less. In a further embodiment, the SLP has a length of 20 to 50, 45 or 40 amino acids, for example from 20 or 25 amino acids to 30, 35 or 40 amino acids.

In another aspect, the disclosure further provides a nucleic acid (isolated) encoding the herein-mentioned TAPs or a tumor antigen precursor-peptide or SLP. In an embodiment, the nucleic acid comprises from about 24 nucleotides to about 1200 nucleotides, from about 24 to about 1000, 900, 800, 700, 600, 500, 400, 300 or 200 nucleotides, for example from about 24 to about 150 or 100 nucleotides, for example 24, 27, 30, 33, 36, 39, 42, 45, 48, 51, 54, 57, 60, 53, 66, 69, or 72 nucleotides. “Isolated”, as used herein, refers to a peptide or nucleic acid molecule separated from other components that are present in the natural environment of the molecule or a naturally occurring source macromolecule (e.g., including other nucleic acids, proteins, lipids, sugars, etc.). “Synthetic”, as used herein, refers to a peptide or nucleic molecule that is not isolated from its natural sources, e.g., which is produced through recombinant technology or using chemical synthesis. In an embodiment, the nucleic acid (DNA, RNA) encoding the TAP or SLP of the disclosure comprises any one of the sequences set forth in the table below or a corresponding RNA sequence. In an embodiment, the nucleic acid encoding the TAP or SLP is an mRNA molecule. In other embodiments, the nucleic acid encoding the TAP or SLP is a self-amplifying mRNA (saRNA), a trans-amplifying mRNA (taRNA) or a circular mRNA (circRNA) (see, e.g., Liu et al., Nature Reviews Cancer, Volume 23, August 2023, pages 526-543).

| | | Nucleotide |
|---|---|---|
| | | SEQ ID |
| TAP sequence | Encoding nucleotide sequence | NO: |
| DELAHFLL | GATGAGTTGGCTCATTTTCTGCTC | 206 |
| DMFGIRFKR | GATATGTTTGGAATAAGGTTCAAAAGA | 152 |
| EADPTGHSY | GAAGCAGACCCCACCGGCCACTCCTAT | 186 |
| EELSVLEVF | GAGGAGCTGAGTGTGTTAGAGGTGTTT | 208 |
| EELSVMEVY | GAGGAGCTGAGTGTGATGGAGGTGTAT | 209 |
| EVDPASNTY | GAAGTGGACCCCGCCAGCAACACCTAC | 187 |
| EVDPTSHSY | GAAGTGGACCCCACTAGCCACTCCTAT | 188 |
| EVYDGREHSAY | GAGGTGTATGATGGGAGGGAGCACAGTGCCTAT | 189 |
| EYNAEFGRR | GAATACAACGCAGAATTTGGCCGAAGG | 153 |
| FPDLESEF | TTCCCTGACCTGGAGTCTGAGTTC | 210 |
| FPDLETSF | TTTCCTGACCTGGAGACGAGCTTC | 207 |
| FPEDFSILF | TTCCCAGAAGACTTTTCAATACTATTT | 154 |
| FVQENYLEY | TTCGTGCAGGAAAACTACCTGGAGTAC | 190 |
| GEALGLVGA | GGAGAGGCCCTGGGCTTGGTGGGTGCG | 191 |
| GEAVGFQPP | GGCGAGGCTGTGGGCTTTCAACCACCA | 155 |
| IYLIPEPVLF | ATTTACCTGATTCCAGAGCCTGTGTTGTTT | 156 |
| LEISFISPL | TTGGAGATAAGTTTCATTTCACCCCTA | 157 |
| LVQEKYLEY | TTGGTGCAGGAAAAGTACCTGGAGTAC | 192 |
| RSMRVGVTR | CGCAGTATGCGGGTGGGAGTGACCCGA | 158 |
| SMRSTVVSR | TCTATGCGCTCCACGGTGGTGTCTCGC | 159 |
| SVLGPILIIPK | AGTGTACTCGGGCCAATATTGATTATACCAAAG | 151 |
| TFLQRKVLR | ACCTTTTTGCAGAGGAAGGTACTGAGG | 193 |
| YEFLWGSKA | TACGAGTTCCTGTGGGGTTCCAAGGCC | 211 |
| SVADLLSSF | TCTGTCGCCGACCTGCTGTCCTCCTTC | 160 |
| AETDITETSI | GCAGAAACTGATATTACAGAAACAAGCATA | 161 |
| DETLTALQF | GATGAAACACTTACTGCTCTCCAGTTT | 194 |
| DIIEYDYEY | GATATAATCGAATATGACTATGAGTAT | 162 |
| EEIFTEEY | GAAGAAATATTTACTGAAGAATAT | 163 |
| EEQYIAQF | GAAGAGCAGTATATCGCCCAGTTC | 164 |
| ELFQHVSLM | GAGCTCTTTCAACATGTCTCCCTGATG | 165 |
| EVFEGDIQQF | GAAGTTTTTGAGGGGGACATTCAGCAGTTT | 195 |
| EVKSITKER | GAAGTGAAATCTATAACCAAAGAAAGA | 196 |
| FAFEKLIQF | TTTGCCTTTGAGAAGCTCATCCAATTT | 197 |
| GAYDKARYL | GGCGCATATGACAAGGCCCGATACCTC | 166 |
| GTHVWVGLYK | GGGACTCATGTTTGGGTAGGCCTGTATAAG | 167 |
| HHNDLVFKF | CATCACAACGACCTGGTTTTCAAATTC | 198 |
| HQFTEKVI | CATCAGTTCACAGAGAAAGTAATC | 168 |
| ITDDQLLAL | ATCACGGATGATCAGCTCCTTGCCCTC | 199 |
| KLKQDTTQK | AAGCTAAAACAAGATACTACTCAGAAA | 169 |
| LEIENLNLA | TTAGAAATAGAGAATTTAAATTTGGCT | 200 |
| LELFQHVSL | CTGGAGCTCTTTCAACATGTCTCCCTG | 170 |
| MAVNIVLQL | ATGGCTGTCAACATCGTGCTGCAGCTG | 171 |
| MLWKLVENV | ATGCTCTGGAAGCTTGTGGAGAATGTC | 172 |
| MPLKHYLLL | ATGCCACTGAAGCATTATCTCCTTTTG | 201 |
| MVFSKLVLR | ATGGTATTCAGCAAATTGGTGTTGAGG | 202 |
| QAWGAGLAY | CAAGCCTGGGGTGCAGGGTTGGCCTAC | 173 |
| QLLALLPSL | CAGCTCCTTGCCCTCCTGCCTTCCCTG | 203 |
| SEADLQAEV | TCTGAAGCAGATCTCCAGGCTGAAGTG | 174 |
| SLPDNVFSNL | TCTCTACCCGACAATGTCTTCAGCAACCTC | 175 |
| SQGQIFSKF | TCTCAAGGCCAAATCTTTTCTAAATTT | 176 |
| SRLELFQHV | TCACGTCTGGAGCTCTTTCAACATGTC | 177 |
| SSQGQIFSK | AGCTCTCAAGGCCAAATCTTTTCTAAA | 178 |
| SVKERESER | TCTGTGAAAGAAAGAGAGAGTGAGAGA | 179 |
| TTLENLPQK | ACCACACTGGAAAACCTGCCCCAGAAG | 204 |
| VEAPHLPSF | GTGGAAGCCCCTCATCTGCCTTCATTC | 205 |
| VEEIFTEEY | GTTGAAGAAATATTTACTGAAGAATAT | 180 |
| VHDPDYAKILL | GTCCATGACCCAGACTATGCCAAGATTCTCCTG | 181 |
| VPWEDKISI | GTTCCCTGGGAGGATAAAATTTCCATT | 182 |
| VTTLALTF | GTAACAACCCTCGCATTGACCTTC | 183 |
| WPMHTPLL | TGGCCCATGCACACCCCACTGCTG | 184 |
| WPMHTPLLL | TGGCCCATGCACACCCCACTGCTGCTG | 185 |

Of course, because of the degeneracy of the genetic code, the TAPs described herein may be encoded by variants of the above-noted sequences.

A nucleic acid of the disclosure may be used for recombinant expression of the TAP or SLP of the disclosure, and may be included in a vector or plasmid, such as a cloning vector or an expression vector, which may be transfected into a host cell. In an embodiment, the disclosure provides a cloning, expression or viral vector or plasmid comprising a nucleic acid sequence encoding the TAP of the disclosure. Alternatively, a nucleic acid encoding a TAP of the disclosure may be incorporated into the genome of the host cell. In either case, the host cell expresses the TAP or protein encoded by the nucleic acid. The term “host cell” as used herein refers not only to the particular subject cell, but to the progeny or potential progeny of such a cell. A host cell can be any prokaryotic (e.g., E. coli) or eukaryotic cell (e.g., insect cells, yeast cells, plant cells, or mammalian cells) capable of expressing the TAPs described herein. The vector or plasmid contains the necessary elements for the transcription and translation of the inserted coding sequence, and may contain other components such as resistance genes, cloning sites, etc. Methods that are well known to those skilled in the art may be used to construct expression vectors containing sequences encoding peptides or polypeptides and appropriate transcriptional and translational control/regulatory elements operably linked thereto. These methods include in vitro recombinant DNA techniques, synthetic techniques, and in vivo genetic recombination. Such techniques are described in Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y. “Operably linked” refers to a juxtaposition of components, particularly nucleotide sequences, such that the normal function of the components can be performed. Thus, a coding sequence that is operably linked to regulatory sequences refers to a configuration of nucleotide sequences wherein the coding sequences can be expressed under the regulatory control, that is, transcriptional and/or translational control, of the regulatory sequences. “Regulatory/control region” or “regulatory/control sequence”, as used herein, refers to the non-coding nucleotide sequences that are involved in the regulation of the expression of a coding nucleic acid. Thus, the term regulatory region includes promoter sequences, regulatory protein binding sites, upstream activator sequences, and the like. The vector (e.g., expression vector) may have the necessary 5′ upstream and 3′ downstream regulatory elements such as promoter sequences such as CMV, PGK and EF-1α promoters, ribosome recognition and binding TATA box, and 3′ UTR AAUAAA transcription termination sequence for the efficient gene transcription and translation in its respective host cell. Other suitable promoters include the constitutive promoter of simian virus 40 (SV40) early promoter, mouse mammary tumor virus (MMTV), HIV LTR promoter, MoMuLV promoter, avian leukemia virus promoter, EBV immediate early promoter, and Rous sarcoma vims promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing the TAP. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Contech), pClneo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. The coding sequences of the TAPs disclosed herein can be ligated into such expression vectors for the expression of the TAP in mammalian cells.

In certain embodiments, the nucleic acids encoding the TAP of the present disclosure are provided in a viral vector. A viral vector can be those derived from adenovirus, vaccinia virus, retrovirus, lentivirus, or foamy virus. As used herein, the term “viral vector” refers to a nucleic acid vector construct that includes at least one element of viral origin and has the capacity to be packaged into a viral vector particle. The viral vector can contain the coding sequence for the various TAPs or SLPs described herein in place of nonessential viral genes. In another embodiment, the nucleic acids encoding the TAP or SLP of the present disclosure are provided in a self-amplifying or self-replicating RNA (saRNA or srRNA) vectors. srRNAs are derived from positive-strand RNA viruses where the structural proteins have been removed and replaced with heterologous genes of interest. srRNAs have been successfully derived from flaviviruses, nodamura viruses, nidoviruses, and alphaviruses with therapeutic versions of the technology providing the structural proteins in trans to create single cycle viral replicon particles (VRPs) (see, e.g., Aliahmad et al. Next generation self-replicating RNA vectors for vaccines and immunotherapies. Cancer Gene Ther (2022). https://doi.org/10.1038/s41417-022-00435-8). The vector and/or particle can be utilized for the purpose of transferring DNA, RNA or other nucleic acids into cells either in vitro or in vivo. Numerous forms of viral vectors are known in the art.

In embodiment, the nucleic acid (DNA, RNA) encoding the TAP or SLP of the disclosure is comprised within a vesicle or nanoparticle such as a lipid vesicle (e.g., liposome) or lipid nanoparticle (LNP), or any other suitable vehicle (e.g., mRNA packaging systems). Thus, in another aspect, the present disclosure provides a vesicle or nanoparticle, such as a lipid vesicle or nanoparticle, comprising a nucleic acid, such as an mRNA, encoding one or more of the TAP or SLP described herein.

The term liposome as used herein in accordance with its usual meaning, referring to microscopic lipid vesicles composed of a bilayer of phospholipids or any similar amphipathic lipids (e.g., sphingolipids) encapsulating an internal aqueous medium.

The term “lipid nanoparticle” refers to liposome-like structure that may include one or more lipid bilayer rings surrounding an internal aqueous medium similar to liposomes, or micellar-like structures that encapsulates molecules (e.g., nucleic acids) in a non-aqueous core. Lipid nanoparticles typically contain cationic lipids, such as ionizable cationic lipids. Examples of cationic lipids that may be used for LNPs include DOTMA, DOSPA, DOTAP, DOPE, ePC, DLin-MC3-DMA, C12-200, ALC-0315, cKK-E12, Lipid H (SM-102), OF-Deg-Lin, A2-Iso5-2DC18, 306Oᵢ₁₀, BAME-O16B, TT3, 9A1P9, FTT5, COATSOME® SS-E, COATSOME® SS-EC, COATSOME® SS-OC and COATSOME® SS-OP (see, e.g., Hou et al., Nature Reviews Materials, volume 6, pages 1078-1094 (2021); Tenchov et al., ACS Nano, 15, 16982-17015 (2021).

Liposomes and lipid nanoparticles typically include other lipid components such as lipids, lipid-like materials, and polymers that can improve liposome or nanoparticle properties, such as stability, delivery efficacy, tolerability and biodistribution. These include phospholipids (e.g., phosphatidylcholines, phosphatidylethanolamines, phosphatidylserines, and phosphatidylglycerol) such as 1,2-distearoyl-sn-glycero-3-phosphocholine (DSPC) and DOPE, sterols (such as cholesterol and cholesterol derivatives), PEGylated lipids (PEG-lipids) such as 1,2-dimyristoyl-rac-glycero-3-methoxypolyethylene glycol-2000 (PEG₂₀₀₀-DMG) and 1,2-distearoyl-rac-glycero-3-methoxypolyethylene glycol-2000 (PEG₂₀₀₀-DSG).

In an embodiment, the lipid nanoparticle according to the present disclosure comprises one or more cationic lipids, such as ionizable cationic lipids. Examples of ionizable cationic lipids include those listed in PCT publications Nos. WO 2017/061150 and WO 2019/188867, which encompassed ionizable cationic lipids commercialized under the tradenames COATSOME® SS-E, COATSOME® SS-EC, COATSOME® SS-OC and COATSOME® SS-OP.

The nucleic acid (e.g., mRNA) encoding one or more of the TAP, may be modified, for example to increase stability and/or reduce immunogenicity. For example, the 5′ end may be capped to stabilize the molecule and decrease immunogenicity (for example, as described in U.S. Pat. Nos. 10,519,189 and 10,494,399). One or more nucleosides of the mRNA may be modified or substituted with 1-methyl pseudo-uridine, pseudouridine (Ψ), N6-methyladenosine, 5-methylcytidine and/or 5-methyluridine to increase stability of the molecule, improve translation efficacy, and/or reduce recognition of the molecule by the innate immune system. A form of modified nucleosides are described in U.S. Pat. No. 9,371,511. Other types of modifications that may be made to the mRNA include incorporation of anti-reverse cap analog (ARCA), 5′-methyl-cytidine triphosphate (m5CTP), N6-methyl-adenosine-5′-triphosphate (m6ATP), 2-thio-uridine triphosphate (s2UTP), pseudouridine triphosphate, N¹Methylpseudouridine triphosphate or 5-Methoxyuridine triphosphate (5moUTP). The mRNA may also include additional modifications to the 5′- and/or 3′-untranslated regions (UTRs) and polyadenylation (polyA) tail (see, for example, Kim et al., Molecular & cellular toxicology vol. 18, 1 (2022): 1-8). The poly(A) tail preferably comprises 100-200 nucleotides, and more preferably 120-150 nucleotides, and may include modified adenosines. All these modifications and other modifications to the nucleic acid (e.g., mRNA) encoding the TAP are encompassed by the present disclosure.

In another aspect, the present disclosure provides an MHC class I molecule comprising (i.e., presenting or bound to) one or more of the BC TAP comprising or consisting of the sequence of SEQ ID NOs:1-61 or SEQ ID NOs:1-35 defined herein.

In an embodiment, the MHC class I molecule is an HLA-A*02:01 molecule. In an embodiment, the MHC class I molecule is an HLA-A*03:01 molecule. In an embodiment, the MHC class I molecule is an HLA-A*11:01 molecule. In an embodiment, the MHC class I molecule is an HLA-A*24:02 molecule. In an embodiment, the MHC class I molecule is an HLA-A*25:01 molecule. In an embodiment, the MHC class I molecule is an HLA-A*26:01 molecule. In an embodiment, the MHC class I molecule is an HLA-A*31:01 molecule. In an embodiment, the MHC class I molecule is an HLA-A*33:01 molecule. In an embodiment, the MHC class I molecule is an HLA-B*15:01 molecule. In an embodiment, the MHC class I molecule is an HLA-B*18:01 molecule. In an embodiment, the MHC class I molecule is an HLA-B*27:05 molecule. In an embodiment, the MHC class I molecule is an HLA-B*35:01 molecule. In an embodiment, the MHC class I molecule is an HLA-B*35:03 molecule. In an embodiment, the MHC class I molecule is an HLA-B*38:01 molecule. In an embodiment, the MHC class I molecule is an HLA-B*40:01 molecule. In an embodiment, the MHC class I molecule is an HLA-B*49:01 molecule. In an embodiment, the MHC class I molecule is an HLA-B*50:01 molecule. In an embodiment, the MHC class I molecule is an HLA-B*51:01 molecule. In an embodiment, the MHC class I molecule is an HLA-B*52:01 molecule. In an embodiment, the MHC class I molecule is an HLA-B*58:01 molecule. In an embodiment, the MHC class I molecule is an HLA-C*01:02 molecule. In an embodiment, the MHC class I molecule is an HLA-C*12:03 molecule.

In an embodiment, the TAP (e.g., comprising or consisting of the sequence of SEQ ID NOs:1-61 or SEQ ID NOs:1-35 defined herein) is non-covalently bound to the MHC class I molecule (i.e., the TAP is loaded into, or non-covalently bound to the peptide binding groove/pocket of the MHC class I molecule). In another embodiment, the TAP is covalently attached/bound to the MHC class I molecule (alpha chain). In such a construct, the TAP and the MHC class I molecule (alpha chain) are produced as a synthetic fusion protein, typically with a short (e.g., 5 to 20 residues, preferably about 8-12, e.g., 10) flexible linker or spacer (e.g., a polyglycine linker). In another aspect, the disclosure provides a nucleic acid encoding a fusion protein comprising a TAP defined herein fused to an MHC class I molecule (alpha chain). In an embodiment, the MHC class I molecule (alpha chain)-peptide complex is multimerized. Accordingly, in another aspect, the present disclosure provides a multimer of MHC class I molecule loaded (covalently or not) with the herein-mentioned TAP. Such multimers may be attached to a tag, for example a fluorescent tag, which allows the detection of the multimers. A great number of strategies have been developed for the production of MHC multimers, including MHC dimers, tetramers, pentamers, octamers, etc. (reviewed in Bakker and Schumacher, Current Opinion in Immunology 2005, 17:428-433). MHC multimers are useful, for example, for the detection and purification of antigen-specific T cells. Thus, in another aspect, the present disclosure provides a method for detecting or purifying (isolating, enriching) CD8⁺ T lymphocytes specific for a TAP defined herein, the method comprising contacting a cell population with a multimer of MHC class I molecule loaded (covalently or not) with the TAP; and detecting or isolating the CD8⁺ T lymphocytes bound by the MHC class I multimers. CD8⁺ T lymphocytes bound by the MHC class I multimers may be isolated using known methods, for example fluorescence activated cell sorting (FACS) or magnetic activated cell sorting (MACS).

In yet another aspect, the present disclosure provides a cell (e.g., a host cell), in an embodiment an isolated cell, comprising the herein-mentioned nucleic acid, vector or plasmid of the disclosure, i.e., a nucleic acid or vector encoding one or more TAPs or SLPs. In another aspect, the present disclosure provides a cell expressing at its surface an MHC class I molecule (e.g., an MHC class I molecule of one of the alleles disclosed above) bound to or presenting a TAP according to the disclosure. In one embodiment, the host cell is a eukaryotic cell, such as a mammalian cell, preferably a human cell. a cell line or an immortalized cell. In another embodiment, the cell is an antigen-presenting cell (APC), such as a dendritic cell. In one embodiment, the host cell is a primary cell, a cell line or an immortalized cell. Nucleic acids and vectors can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), and other laboratory manuals. Methods for introducing nucleic acids into mammalian cells in vivo are also known, and may be used to deliver the vector or plasmid of the disclosure to a subject for gene therapy.

Cells such as APCs can be loaded with one or more TAPs using a variety of methods known in the art. As used herein “loading a cell” with a TAP means that RNA or DNA encoding the TAP, or the TAP, is transfected into the cells or alternatively that the APC is transformed with a nucleic acid encoding the TAP. The cell can also be loaded by contacting the cell with exogenous TAPs that can bind directly to MHC class I molecule present at the cell surface (e.g., peptide-pulsed cells). The TAPs may also be fused to a domain or motif that facilitates its presentation by MHC class I molecules, for example to an endoplasmic reticulum (ER) retrieval signal, a C-terminal Lys-Asp-Glu-Leu sequence (see Wang et al., Eur J Immunol. 2004 December; 34(12):3582-94).

In another aspect, the present disclosure provides a composition or peptide combination/pool comprising any one of, or any combination of, the TAPs defined herein (or a nucleic acid encoding said peptide(s)). In an embodiment, the composition comprises any combination of the TAPs defined herein (any combination of 2, 3, 4, 5, 6, 7, 8, 9, 10 or more TAPs), or a combination of nucleic acids encoding said TAPs). Compositions comprising any combination/sub-combination of the TAPs defined herein are encompassed by the present disclosure. In another embodiment, the combination or pool may comprise one or more known tumor antigens.

Thus, in another aspect, the present disclosure provides a composition comprising any one of, or any combination of, the TAPs defined herein (e.g., comprising or consisting of the sequence of SEQ ID NOs:1-61 or SEQ ID NOs:1-35 defined herein) and a cell expressing a MHC class I molecule (e.g., a MHC class I molecule of one of the alleles disclosed above). APC for use in the present disclosure are not limited to a particular type of cell and include professional APCs such as dendritic cells (DCs), Langerhans cells, macrophages and B cells, which are known to present proteinaceous antigens on their cell surface so as to be recognized by CD8⁺ T lymphocytes. For example, an APC can be obtained by inducing DCs from peripheral blood monocytes and then contacting (stimulating) the TAPs, either in vitro, ex vivo or in vivo. APC can also be activated to present a TAP in vivo where one or more of the TAPs of the disclosure are administered to a subject and APCs that present a TAP are induced in the body of the subject. The phrase “inducing an APC” or “stimulating an APC” includes contacting or loading a cell with one or more TAPs, or nucleic acids encoding the TAPs such that the TAPs are presented at its surface by MHC class I molecules. As noted herein, according to the present disclosure, the TAPs may be loaded indirectly for example using longer peptides/polypeptides comprising the sequence of the TAPs (including the native protein), which is then processed (e.g., by proteases) inside the APCs to generate the TAP/MHC class I complexes at the surface of the cells. After loading APCs with TAPs and allowing the APCs to present the TAPs, the APCs can be administered to a subject as a vaccine. For example, the ex vivo administration can include the steps of: (a) collecting APCs from a first subject, (b) contacting/loading the APCs of step (a) with a TAP to form MHC class I/TAP complexes at the surface of the APCs; and (c) administering the peptide-loaded APCs to a second subject in need for treatment.

The first subject and the second subject may be the same subject (e.g., autologous vaccine), or may be different subjects (e.g., allogeneic vaccine). Alternatively, according to the present disclosure, use of a TAP described herein (or a combination thereof) for manufacturing a composition (e.g., a pharmaceutical composition) for inducing antigen-presenting cells is provided. In addition, the present disclosure provides a method or process for manufacturing a pharmaceutical composition for inducing antigen-presenting cells, wherein the method or the process includes the step of admixing or formulating the TAP, or a combination thereof, with a pharmaceutically acceptable carrier. Cells such as APCs expressing a MHC class I molecule (e.g., any of the above-noted HLA molecules) loaded with any one of, or any combination of, the TAPs defined herein, may be used for stimulating/amplifying CD8⁺ T lymphocytes, for example autologous CD8⁺ T lymphocytes. Accordingly, in another aspect, the present disclosure provides a composition comprising any one of, or any combination of, the TAPs defined herein (or a nucleic acid or vector encoding same); a cell expressing an MHC class I molecule and a T lymphocyte, more specifically a CD8⁺ T lymphocyte (e.g., a population of cells comprising CD8⁺ T lymphocytes).

In an embodiment, the composition further comprises a buffer, an excipient, a carrier, a diluent and/or a medium (e.g., a culture medium). In a further embodiment, the buffer, excipient, carrier, diluent and/or medium is/are pharmaceutically acceptable buffer(s), excipient(s), carrier(s), diluent(s) and/or medium (media). As used herein “pharmaceutically acceptable buffer, excipient, carrier, diluent and/or medium” includes any and all solvents, buffers, binders, lubricants, fillers, thickening agents, disintegrants, plasticizers, coatings, barrier layer formulations, lubricants, stabilizing agent, release-delaying agents, dispersion media, coatings, antibacterial and antifungal agents, isotonic agents, and the like that are physiologically compatible, do not interfere with effectiveness of the biological activity of the active ingredient(s) and that are not toxic to the subject. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound (peptides, cells), use thereof in the compositions of the disclosure is contemplated. In an embodiment, the buffer, excipient, carrier and/or medium is a non-naturally occurring buffer, excipient, carrier and/or medium. In an embodiment, one or more of the TAPs defined herein, or the nucleic acids (e.g., mRNAs) encoding said one or more TAPs, are comprised within or complexed to a lipid vesicle or liposome, e.g., a cationic liposome (see, e.g., Vitor M T et al., Recent Pat Drug Deliv Formul. 2013 August; 7(2):99-110) or suitable other carriers.

In another aspect, the present disclosure provides a composition comprising one of more of the any one of, or any combination of, the TAPs defined herein (e.g., comprising or consisting of the sequence of SEQ ID NOs:1-61 or SEQ ID NOs:1-35 defined herein) (or a nucleic acid such as a mRNA encoding said peptide(s)), and a buffer, an excipient, a carrier, a diluent and/or a medium. For compositions comprising cells (e.g., APCs, T lymphocytes), the composition comprises a suitable medium that allows the maintenance of viable cells. Representative examples of such media include saline solution, Earl's Balanced Salt Solution (Life Technologies®) or PlasmaLyte® (Baxter International®). In an embodiment, the composition (e.g., pharmaceutical composition) is an “immunogenic composition”, “vaccine composition” or “vaccine”. The term “Immunogenic composition”, “vaccine composition” or “vaccine” as used herein refers to a composition or formulation comprising one or more TAPs, nucleic acids or vaccine vector and which is capable of inducing an immune response against the one or more TAPs present therein when administered to a subject. Vaccination methods for inducing an immune response in a mammal (e.g., human) comprise use of a vaccine or vaccine vector to be administered by any conventional route known in the vaccine field, e.g., via a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g., via a transdermal delivery system such as a patch). In an embodiment, the TAP (or a combination thereof) is conjugated to a carrier protein (conjugate vaccine) to increase the immunogenicity of the TAP(s). The present disclosure thus provides a composition (conjugate) comprising a TAP (or a combination thereof), or a nucleic acid encoding the TAP or combination thereof, and a carrier protein. For example, the TAP(s) or nucleic acid(s) may be conjugated or complexed to a Toll-like receptor (TLR) ligand (see, e.g., Zom et al., Adv Immunol. 2012, 114: 177-201) or polymers/dendrimers (see, e.g., Liu et al., Biomacromolecules. 2013 Aug. 12; 14(8):2798-806) such as polymer-linked TLR agonists (see, e.g., Lynn et al., Nature Biotechnology 33: 1201-1210 (2015); Lynn et al., Nature Biotechnology 38: 320-332 (2020)). In an embodiment, the immunogenic composition or vaccine further comprises an adjuvant. “Adjuvant” refers to a substance which, when added to an immunogenic agent such as an antigen (TAPs, nucleic acids and/or cells according to the present disclosure), nonspecifically enhances or potentiates an immune response to the agent in the host upon exposure to the mixture. Examples of adjuvants currently used in the field of vaccines include (1) mineral salts (aluminum salts such as aluminum phosphate and aluminum hydroxide, calcium phosphate gels), squalene, (2) oil-based adjuvants such as oil emulsions and surfactant based formulations, e.g., MF59 (microfluidised detergent stabilised oil-in-water emulsion), QS21 (purified saponin), AS02 [SBAS2] (oil-in-water emulsion+MPL+QS-21), (3) particulate adjuvants, e.g., virosomes (unilamellar liposomal vehicles incorporating influenza haemagglutinin), AS04 ([SBAS4] aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL+M. phlei cell wall skeleton), AGP [RC-529] (synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified Cholera toxin (CT) and Escherichia coli enterotoxin (LT) (genetically modified bacterial toxins to provide non-toxic adjuvant effects), (5) endogenous human immunomodulators, e.g., hGM-CSF or hIL-12 (cytokines that can be administered either as protein or plasmid encoded), Immudaptin (C3d tandem array) and/or (6) inert vehicles, such as gold particles, and the like. In an embodiment, the vaccine is an RNA vaccine.

In an embodiment, the TAP(s) (e.g., comprising or consisting of the sequence of SEQ ID NOs:1-61 or SEQ ID NOs:1-35), or SLPs (or a nucleic acid such as a mRNA encoding said peptide(s)) or composition comprising same is/are in lyophilized form. In another embodiment, the TAP(s), SLP(s), nucleic acid(s) or composition comprising same is/are in a liquid composition. In a further embodiment, the TAP(s) or nucleic acid(s) is/are at a concentration of about 0.01 μg/mL to about 100 μg/mL in the composition. In further embodiments, the TAP(s) or nucleic acid(s) is/are at a concentration of about 0.2 μg/mL to about 50 μg/mL, about 0.5 μg/mL to about 10, 20, 30, 40 or 50 μg/mL, about 1 μg/mL to about 10 μg/mL, or about 2 μg/mL, in the composition.

As noted herein, cells such as APCs that express an MHC class I molecule loaded with or bound to any one of, or any combination of, the TAPs defined herein, may be used for stimulating/amplifying CD8⁺ T lymphocytes in vivo or ex vivo. Accordingly, in another aspect, the present disclosure provides T cell receptor (TCR) molecules capable of interacting with or binding the herein-mentioned MHC class I molecule/TAP complex, and nucleic acid molecules encoding such TCR molecules, and vectors comprising such nucleic acid molecules. A TCR according to the present disclosure is capable of specifically interacting with or binding a TAP loaded on, or presented by, an MHC class I molecule, preferably at the surface of a living cell in vitro or in vivo.

The term TCR as used herein refers to an immunoglobulin superfamily member having a variable binding domain, a constant domain, a transmembrane region, and a short cytoplasmic tail; see, e.g., Janeway et al, Immunobiology: The Immune System in Health and Disease, 3rd Ed., Current Biology Publications, p. 4:33, 1997) capable of specifically binding to an antigen peptide bound to a MHC receptor. A TCR can be found on the surface of a cell and generally is comprised of a heterodimer having a and p chains (also known as TCRα and TCRβ, respectively). Like immunoglobulins, the extracellular portion of TCR chains (e.g., α-chain, β-chain) contain two immunoglobulin regions, a variable region (e.g., TCR variable α region or Vα and TCR variable β region or Vβ; typically amino acids 1 to 116 based on Rabat numbering at the N-terminus), and one constant region (e.g., TCR constant domain α or Cα and typically amino acids 117 to 259 based on Rabat, TCR constant domain β or Cβ, typically amino acids 117 to 295 based on Rabat) adjacent to the cell membrane. Also, like immunoglobulins, the variable domains contain complementary determining regions (CDRs. 3 in each chain) separated by framework regions (FRs). In certain embodiments, a TCR is found on the surface of T cells (or T lymphocytes) and associates with the CD3 complex.

A TCR and in particular nucleic acids encoding a TCR of the disclosure may for instance be applied to genetically transform/modify T lymphocytes (e.g., CD8⁺ T lymphocytes) or other types of lymphocytes generating new T lymphocyte clones that specifically recognize an MHC class I/TAP complex. In a particular embodiment, T lymphocytes (e.g., CD8⁺ T lymphocytes) obtained from a patient are transformed to express one or more TCRs that recognize a TAP and the transformed cells are administered to the patient (autologous cell transfusion). In a particular embodiment, T lymphocytes (e.g., CD8⁺ T lymphocytes) obtained from a donor are transformed to express one or more TCRs that recognize a TAP and the transformed cells are administered to a recipient (allogenic cell transfusion). In another embodiment, the disclosure provides a T lymphocyte e.g., a CD8⁺ T lymphocyte transformed/transfected by a vector or plasmid encoding a TAP-specific TCR. In a further embodiment the disclosure provides a method of treating a patient with autologous or allogenic cells transformed with a TAP-specific TCR. In certain embodiments, TCRs are expressed in primary T cells (e.g., cytotoxic T cells) by replacing an endogenous locus, e.g., an endogenous TRAC and/or TRBC locus, using, e.g., CRISPR, TALEN, zinc finger nuclease, or other targeted disruption systems.

In another embodiment, the present disclosure provides a nucleic acid encoding the above-noted TCR. In a further embodiment, the nucleic acid is present in a vector, such as the vectors described above.

In yet a further embodiment the use of a tumor antigen-specific TCR in the manufacture of autologous or allogenic cells for the treatment of breast cancer is provided.

In some embodiments, patients treated with the compositions (e.g., pharmaceutical compositions) of the disclosure are treated prior to or following treatment with an anti-tumor agent and/or immunotherapy (e.g., CAR therapy, immune checkpoint inhibitor therapy). Compositions of the disclosure include: allogenic T lymphocytes (e.g., CD8⁺ T lymphocyte) activated ex vivo against a TAP; allogenic or autologous APC vaccines loaded with a TAP; vaccines including TAPs of nucleic acids (e.g. mRNA) encoding TAPs and allogenic or autologous T lymphocytes (e.g., CD8⁺ T lymphocyte) or lymphocytes transformed with a tumor antigen-specific TCR. The method to provide T lymphocyte clones capable of recognizing a TAP according to the disclosure may be generated for and can be specifically targeted to tumor cells expressing the TAP in a subject (e.g., graft recipient), for example an allogenic T lymphocyte and/or donor lymphocyte infusion (DLI) recipient. Hence the disclosure provides a CD8⁺ T lymphocyte encoding and expressing a T cell receptor capable of specifically recognizing or binding a TAP/MHC class I molecule complex. Said T lymphocyte (e.g., CD8⁺ T lymphocyte) may be a recombinant (engineered) or a naturally selected T lymphocyte. This specification thus provides at least two methods for producing CD8⁺ T lymphocytes of the disclosure, comprising the step of bringing undifferentiated lymphocytes into contact with a TAP/MHC class I molecule complex (typically expressed at the surface of cells, such as APCs) under conditions conducive of triggering T cell activation and expansion, which may be done in vitro or in vivo (i.e. in a patient administered with a APC vaccine wherein the APC is loaded with a TAP or in a patient treated with a TAP vaccine). Using a combination or pool of TAPs bound to MHC class I molecules, it is possible to generate a population CD8⁺ T lymphocytes capable of recognizing a plurality of TAPs. Alternatively, tumor antigen-specific or targeted T lymphocytes may be produced/generated in vitro or ex vivo by cloning one or more nucleic acids (genes) encoding a TCR (more specifically the alpha and beta chains) that specifically binds to a MHC class I molecule/TAP complex (i.e. engineered or recombinant CD8⁺ T lymphocytes). Nucleic acids encoding a TAP-specific TCR of the disclosure, may be obtained using methods known in the art from a T lymphocyte activated against a TAP ex vivo (e.g., with an APC loaded with a TAP); or from an individual exhibiting an immune response against peptide/MHC molecule complex. TAP-specific TCRs of the disclosure may be recombinantly expressed in a host cell and/or a host lymphocyte obtained from a graft recipient or graft donor, and optionally differentiated in vitro to provide cytotoxic T lymphocytes (CTLs). The nucleic acid(s) (transgene(s)) encoding the TCR alpha and beta chains may be introduced into a T cells (e.g., from a subject to be treated or another individual) using any suitable methods such as transfection (e.g., electroporation) or transduction (e.g., using viral vector). The engineered CD8⁺ T lymphocytes expressing a TCR specific for a TAP may be expanded in vitro using well known culturing methods.

The present disclosure provides methods for making the immune effector cells which express the TCRs as described herein. In one embodiment, the method comprises transfecting or transducing immune effector cells, e.g., immune effector cells isolated from a subject, such as a subject having a breast cancer, such that the immune effector cells express one or more TCR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a TCR. In this regard, the immune effector cells may be cultured before or after being genetically modified (i.e., transduced or transfected to express a TCR as described herein).

Prior to in vitro manipulation or genetic modification of the immune effector cells described herein, the source of cells may be obtained from a subject. In particular, the immune effector cells for use with the TCRs as described herein comprise T cells. T cells can be obtained from a number of sources, including peripheral blood mononuclear cells (PBMCs), bone marrow, lymph nodes tissue, cord blood, thymus issue, tissue from a site of infection, ascites, pleural effusion, spleen tissue, and tumors. In certain embodiments, T cell can be obtained from a unit of blood collected from the subject using any number of techniques known to the skilled person, such as FICOLL™ separation. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. In one embodiment of the invention, the cells are washed with PBS. In an alternative embodiment, the washed solution lacks calcium and may lack magnesium or may lack many if not all divalent cations. As would be appreciated by those of ordinary skill in the art, a washing step may be accomplished by methods known to those in the art, such as by using a semi-automated flow-through centrifuge. After washing, the cells may be resuspended in a variety of biocompatible buffers or other saline solution with or without buffer. In certain embodiments, the undesirable components of the apheresis sample may be removed in the cell directly resuspended culture media. In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28⁺, CD4⁺, CD8⁺, CD45RA⁺, and CD45RO⁺ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD8+ cells by negative selection, a monoclonal antibody cocktail typically includes antibodies to CD14, CD20, CD11b, CD16, HLA-DR, and CD4. Flow cytometry and cell sorting may also be used to isolate cell populations of interest for use in the present disclosure. PBMC may be used directly for genetic modification with the TCRs using methods as described herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion.

The present disclosure provides isolated immune cells such as T lymphocytes (e.g., CD8⁺ T lymphocytes) that are specifically induced, activated and/or amplified (expanded) by a TAP (i.e., a TAP bound to MHC class I molecules expressed at the surface of cell), or a combination of TAPs. The present disclosure also provides a composition comprising CD8⁺ T lymphocytes capable of recognizing a TAP, or a combination thereof, according to the disclosure (i.e., one or more TAPs bound to MHC class I molecules) and said TAP(s). In another aspect, the present disclosure provides a cell population or cell culture (e.g., a CD8⁺ T lymphocyte population) enriched in T lymphocytes (e.g., CD8⁺ T lymphocytes) that specifically recognize one or more MHC class I molecule/TAP complex(es) as described herein. Such enriched population may be obtained by performing an ex vivo expansion of specific T lymphocytes using cells such as APCs that express MHC class I molecules loaded with (e.g., presenting) one or more of the TAPs disclosed herein. “Enriched” as used herein means that the proportion of tumor antigen-specific T lymphocytes (e.g., CD8⁺ T lymphocytes) in the population is significantly higher relative to a native population of cells, i.e., which has not been subjected to a step of ex vivo-expansion of specific T lymphocytes. In a further embodiment, the proportion of TAP-specific T lymphocytes (e.g., CD8⁺ T lymphocytes) in the cell population is at least about 0.5%, for example at least about 1%, 1.5%, 2% or 3%. In some embodiments, the proportion of TAP-specific T lymphocytes (e.g., CD8⁺ T lymphocytes) in the cell population is about 0.5 to about 10%, about 0.5 to about 8%, about 0.5 to about 5%, about 0.5 to about 4%, about 0.5 to about 3%, about 1% to about 5%, about 1% to about 4%, about 1% to about 3%, about 2% to about 5%, about 2% to about 4%, about 2% to about 3%, about 3% to about 5% or about 3% to about 4%. Such cell population or culture (e.g., a CD8⁺ T lymphocyte population) enriched in T lymphocytes (e.g., CD8⁺ T lymphocytes) that specifically recognizes one or more MHC class I molecule/peptide (TAP) complex(es) of interest may be used in tumor antigen-based cancer immunotherapy, as detailed below. In some embodiments, the population of TAP-specific T lymphocytes (e.g., CD8⁺ T lymphocytes) is further enriched, for example using affinity-based systems such as multimers of MHC class I molecule loaded (covalently or not) with the TAP(s) defined herein. Thus, the present disclosure provides a purified or isolated population of TAP-specific T lymphocytes (e.g., CD8⁺ T lymphocytes), e.g., in which the proportion of TAP-specific T lymphocytes (e.g., CD8⁺ T lymphocytes) is at least about 50%, 60%, 70%, 80%, 85%, 90%, 95%, 96%, 97%, 98%, 99% or 100%.

In another aspect, the present disclosure provides an antibody or an antigen-binding fragment thereof (e.g., a TCR mimic or TCR-like antibody), or a soluble TCR, that specifically binds to a complex comprising a TAP as described herein bound to an HLA molecule, such as the HLA molecules defined herein. The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity. Antibody fragments comprise a portion of a full-length antibody, generally an antigen binding or variable region thereof. Examples of antibody fragments include Fab, Fab′, F(ab′)₂, and Fv fragments, diabodies, linear antibodies, single-chain antibody molecules (e.g., single-chain Fv, scFv), single domain antibodies (e.g., from camelids), shark NAR single domain antibodies, and multispecific antibodies formed from antibody fragments, single-chain diabodies (scDbs), bispecific T cell engagers (BiTEs), dual affinity retargeting molecules (DARTs), bivalent scFv-Fcs, and trivalent scFv-Fcs. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies. In an embodiment, the antibody or antigen-binding fragment thereof is a single-chain antibody, preferably a single-chain Fv (scFv). In an embodiment, the antibody or antigen-binding fragment thereof comprises at least one constant domain, e.g., a constant domain of a light and/or heavy chain, or a fragment thereof. In a further embodiment, the antibody or antigen-binding fragment thereof comprises a Fragment crystallizable (Fc) fragment of the constant heavy chain of an antibody. In an embodiment, the antibody or antigen-binding fragment is a scFv comprising a Fc fragment (scFV-Fc). In an embodiment, the scFv component is connected to the Fc fragment by a linker, for example a hinge. The presence of an Fc region is useful to induce a complement-dependent cytotoxicity (CDC), antibody-dependent cellular phagocytosis (ADCP), or antibody-dependent cellular cytotoxicity (ADCC) response against a tumor cell.

In an embodiment, the antibody or antigen-binding fragment thereof is a multispecific antibody or an antigen-binding fragment thereof, such as a bispecific antibody or an antigen-binding fragment thereof, wherein at least one of the antigen-binding domains of the multispecific antibody or antibody fragment recognize(s) a complex comprising a TAP as described herein bound to an HLA molecule. In an embodiment, at least one of the antigen-binding domains of the multispecific antibody or antibody fragment recognize(s) an immune cell effector molecule. The term “immune cell effector molecule” refers to a molecule (e.g., protein) expressed by an immune cell and whose engagement by the multispecific antibody or antibody fragment leads to activation of the immune cells. Examples of immune cell effector molecules include the CD3 signaling complex in T cells such as CD8 T cells and the various activating receptors on NK cells (NKG2D, KIR2DS, NKp44, etc.). In a further embodiment, at least one of the antigen-binding domains of the multispecific antibody or antibody fragment recognize(s) and engage(s) the CD3 signaling complex in T cells (e.g., anti-CD3). In a further embodiment, the multispecific antibody or antibody fragment is a single-chain diabody (scDb). In a further embodiment, the scDb comprises a first antibody fragment (e.g., scFv) that binds to a complex comprising a TAP as described herein bound to an HLA molecule and a second antibody fragment (e.g., scFv) that binds to and engages an immune cell effector molecule, such as the CD3 signaling complex in T cells (e.g., anti-CD3 scFv). Such constructs may be used for example to induce the cytotoxic T cell-mediated killing of tumor cells expressing the tumor antigen/MHC complex recognized by the multispecific antibody or antibody fragment. Antibodies or antigen-binding fragments thereof may also be used as a chimeric antigen receptor (CAR) to produce CAR T cells, CAR NK cells, etc. CAR combines a ligand-binding domain (e.g., antibody or antibody fragment) that provides specificity for a desired antigen (e.g., MHC/TAP complex) with an activating intracellular domain (or signal transducing domain) portion, such as a T cell or NK cell activating domain, providing a primary activation signal. Antigen-binding fragments of antibodies, and more particularly scFv, capable of binding to molecules expressed by tumor cells are commonly used as ligand-binding domains in CAR.

In an embodiment, the soluble TCR is a soluble therapeutic bispecific TCR (see, e.g., Robinson et al., FEBS J. 2021 November; 288(21):6159-6173; Dilchert et al., Antibodies (Basel). 2022 May 10; 11(2):34).

In an embodiment, the soluble TCR, antibody or antibody fragment (e.g., TCR-mimic) is attached to an antitumor agent to form an antibody-drug conjugate (ADC). Such ADC permits to target the antitumor agent to tumor cells expressing one or more of the TAPs described herein (see, e.g., Shen et al., Asian J Pharm Sci. 2020 November; 15(6):777-785).

The present disclosure also provides a nucleic acid such as an mRNA encoding the soluble TCR, antibody, antibody fragment or CAR described herein. Such nucleic acids may be formulated into suitable vehicles such as lipid nanoparticles are described above, and may be used in the treatment of cancers such as breast cancer, as described below

Thus, in another aspect, the present disclosure provides a host cell, preferably an immune cell such as a T cell or NK cell, expressing the antibody or antibody fragment (e.g., scFv) described herein.

The present disclosure further relates to a pharmaceutical composition or vaccine comprising the above-noted immune cell (CD8⁺ T lymphocytes, CAR T cell) or population of TAP-specific CD8⁺ T lymphocytes. Such pharmaceutical composition or vaccine may comprise one or more pharmaceutically acceptable excipients and/or adjuvants, as described above.

In another aspect, the present disclosure further relates to the use of any of the TAP comprising or consisting of any of the sequences of SEQ ID NOs:1-61 or SEQ ID NOs:1-35, nucleic acid, expression vector, T cell receptor, antibody/antibody fragment, cell (e.g., T lymphocyte, APC, CAR T cell), and/or composition according to the present disclosure, or any combination thereof, as a medicament or in the manufacture of a medicament for the treatment of breast cancer. The present disclosure relates to any TAP, nucleic acid, expression vector, T cell receptor, antibody/antibody fragment, cell (e.g., T lymphocyte, APC), and/or composition (e.g., vaccine composition) according to the present disclosure, or any combination thereof, for use in the treatment of breast cancer e.g., as a breast cancer vaccine. The TAP sequences identified herein may be used for the production of synthetic peptides to be used i) for in vitro priming and expansion of tumor antigen-specific T cells to be injected into tumor patients and/or ii) as vaccines to induce or boost the anti-tumor T cell response in breast cancer patients.

In another aspect, the present disclosure provides the use of a TAP or SLP described herein (e.g., comprising or consisting of any of the sequences of SEQ ID NOs:1-61 or SEQ ID NOs:1-35), or a combination thereof (e.g., a peptide pool), or of one or more nucleic acid(s) encoding the TAP(s) or SLP(s), as a vaccine for treating breast cancer in a subject. The present disclosure also provides the TAP or SLP described herein, or a combination thereof (e.g., a peptide pool), or of one or more nucleic acid(s) encoding the TAP(s) or SLP(s), for use as a vaccine for treating breast cancer in a subject. In an embodiment, the subject is a recipient of TAP-specific T lymphocytes (e.g., CD8⁺ T lymphocytes). Accordingly, in another aspect, the present disclosure provides a method of treating breast cancer (e.g., of reducing the number of tumor cells, killing tumor cells), said method comprising administering (infusing) to a subject in need thereof an effective amount of T lymphocytes (e.g., CD8⁺ T lymphocytes) recognizing (i.e., expressing a TCR that binds) one or more MHC class I molecule/TAP complexes (expressed at the surface of a cell such as an APC). In an embodiment, the method further comprises administering an effective amount of the TAP or SLP, or a combination thereof, or of one or more nucleic acid(s) encoding the TAP(s), and/or a cell (e.g., an APC such as a dendritic cell) expressing MHC class I molecule(s) loaded with the TAP(s) or SLP(s), to said subject after administration/infusion of said CD8⁺ T lymphocytes. In yet a further embodiment, the method comprises administering to a subject in need thereof a therapeutically effective amount of a dendritic cell loaded with one or more TAPs. In yet a further embodiment the method comprises administering to a patient in need thereof a therapeutically effective amount of an allogenic or autologous cell that expresses a recombinant TCR that binds to a TAP presented by an MHC class I molecule.

In another aspect, the present disclosure provides the use of T lymphocytes (e.g., CD8⁺ T lymphocytes) that recognize one or more MHC class I molecules loaded with (presenting) a TAP, or a combination thereof, for treating breast cancer (e.g., of reducing the number of tumor cells, killing tumor cells) in a subject. In another aspect, the present disclosure provides the use of T lymphocytes (e.g., CD8⁺ T lymphocytes) that recognize one or more MHC class I molecules loaded with (presenting) a TAP, or a combination thereof, for the preparation/manufacture of a medicament for treating breast cancer (e.g., for reducing the number of tumor cells, killing tumor cells), such as a lymphoblastic leukemia, in a subject. In another aspect, the present disclosure provides T lymphocytes (e.g., CD8⁺ T lymphocytes) that recognize one or more MHC class I molecule(s) loaded with (presenting) a TAP, or a combination thereof, for use in the treatment of breast cancer (e.g., for reducing the number of tumor cells, killing tumor cells), in a subject. In a further embodiment, the use further comprises the use of an effective amount of a TAP (or a combination thereof), or of one or more nucleic acid(s) encoding the TAP(s), and/or of a cell (e.g., an APC) that expresses one or more MHC class I molecule(s) loaded with (presenting) a TAP, after the use of said TAP-specific T lymphocytes.

The present disclosure also provides a method of generating an immune response against tumor cells expressing human class I MHC molecules loaded with any of the TAP disclosed herein (e.g., comprising or consisting of any of the sequences of SEQ ID NOs:1-61 or SEQ ID NOs:1-35) or combination thereof in a subject, the method comprising administering cytotoxic T lymphocytes that specifically recognizes the class I MHC molecules loaded with the TAP or combination of TAPs. The present disclosure also provides the use of cytotoxic T lymphocytes that specifically recognizes class I MHC molecules loaded with any of the TAP or combination of TAPs disclosed herein for generating an immune response against tumor cells expressing the human class I MHC molecules loaded with the TAP or combination thereof.

The breast cancer may be a ductal carcinoma in situ (DCIS), an invasive ductal carcinoma (IDS) or an invasive lobular carcinoma (ILC). The breast cancer may be a hormone receptor positive (HR+) breast cancer, or a breast cancer in which one or more of the estrogen receptor (ER), progesterone receptor (PR) or epidermal growth factor receptor (HER2) is not expressed or is expressed at very low levels (undetectable by standard tests). In an embodiment, the breast cancer is a triple-negative breast cancer (TNBC) in which all of the receptors (ER, PR and HER2) are not expressed or are expressed at very low levels. The breast cancer may be at any stage, i.e., in situ breast cancer (stage 0), early stage (1A, 1B or 2A), locally advanced breast cancer (2B, 3A, 3B or 3C), or metastatic breast cancer (stage 4).

In an embodiment, the methods or uses described herein further comprise determining the HLA class I alleles expressed by the patient prior to the treatment/use, and administering or using TAPs that bind to one or more of the HLA class I alleles expressed by the patient. For example, if it is determined that the patient expresses HLA-11*01 and HLA-B35*01, any combinations of (i) the TAPs of SEQ ID NO: SEQ ID NO:1, 17 and/or 28 (that bind to HLA-A11*01) and (ii) the TAPs of SEQ ID NO:4, 12 and/or 23 (that bind to HLA-B35*01) may be administered or used in the patient.

In an embodiment, the TAP, nucleic acid, expression vector, T cell receptor, antibody/antibody fragment, cell (e.g., T lymphocyte, CAR T or NK cell, APC), and/or composition according to the present disclosure, or any combination thereof, may be used in combination with one or more additional active agents or therapies to treat breast cancer, such as chemotherapy (e.g., vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof), geldanamycin or a derivative thereof (such as 17-AAG), inhibitors of CDK4/6, TGF-β, WNT-β-catenin, MYC or PI3K, surgery, immune checkpoint inhibitors or immunotherapeutic agents (e.g., PD-1/PD-L1 inhibitors such as anti-PD-1/PD-L1 antibodies, CTLA-4 inhibitors such as anti-CTLA-4 antibodies, B7-1/B7-2 inhibitors such as anti-B7-1/B7-2 antibodies, TIM3 inhibitors such as anti-TIM3 antibodies, BTLA inhibitors such as anti-BTLA antibodies, CD47 inhibitors such as anti-CD47 antibodies, GITR inhibitors such as anti-GITR antibodies), antibodies against tumor antigens (e.g., anti-CD19, anti-CD22 antibodies), cell-based therapies (e.g., CAR T cells, CAR NK cells), and cytokines such as IL-2, IL-7, IL-21, and IL-15. In an embodiment, the TAP, nucleic acid, expression vector, T cell receptor, cell (e.g., T lymphocyte, APC), and/or composition according to the present disclosure is administered/used in combination with an immune checkpoint inhibitor. In an embodiment, the TAP, nucleic acid, expression vector, T cell receptor, cell (e.g., T lymphocyte, APC), and/or composition according to the present disclosure is administered/used in combination with inhibitors of CDK4/6, TGF-β and/or WNT-β-catenin. Several CDK4/6 inhibitors are in clinical trials including Palbociclib (PD-0332991, Ibrance), Ribociclib (LEE-011, Kisqali), Abemaciclib (LY2835219, Verzenios), SHR6390 and Trilaciclib (G1T28). Inhibitors of TGF-β include antisense inhibitors such as AP12009 (Trabedersen) and ISTH0036, antibodies and ligand traps such as GC1008 (Fresolimumab), LY2382770, and P144, vaccines targeting the TGF-β pathway such as Belagenpumatucel-L (Lucanix™), and FANG™ or vigil (Gemogenovatucel-T), as well as small molecule inhibitors such as LY2157299 (Galunisertib) and TEW-7197. Inhibitors of the WNT-β-catenin pathway include amino acid starvators (asparaginase), GSK3 inhibitors, C2

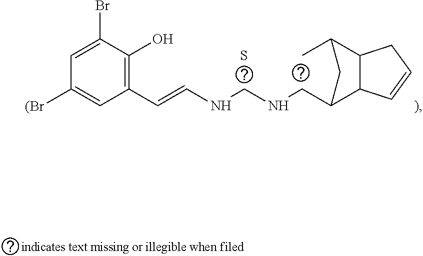

WNT974, ETC-1922159, RXC004, CGX1321, OTSA101-DTPA-90Y, Vantictumab (OMP-18R5), Ipafricept (OMP-54F28), PRI-724, SM08502, secreted frizzled-related proteins/peptides and Tankyrase inhibitors (XAV939, JW-55, RK-287107, and G007-LK).

The additional therapy may be administered prior to, concurrent with, or after the administration of the TAP, nucleic acid, expression vector, T cell receptor, antibody/antibody fragment, cell (e.g., T lymphocyte, CAR T or NK cell, APC), and/or composition according to the present disclosure.

### EXAMPLES

The present disclosure is illustrated in further details by the following non-limiting examples.

### Example 1: Materials and Methods

Primary breast cancer samples. Fresh frozen primary breast tumor samples were bought from Tissue solutions (https://www.tissue-solutions.com/). Samples had a histological diagnosis of invasive ductal carcinoma (n=24) or invasive carcinoma (n=2). Immunohistochemistry data was available through Tissue solutions, and samples were categorized as HR⁺ (ER or PR positive and HER2 negative; n=14) or TNBC (ER/PR/HER2 negative; n=12). Patients did not receive chemotherapy before resection.

RNA and miRNA sequencing. Total RNA was isolated using an RNeasy™ mini kit (Qiagen) according to the manufacturer's instructions. RNA was quantified using Qubit™ (Thermo Scientific), and quality was assessed with the 2100 Bioanalyzer™ (Agilent Technologies). Transcriptome libraries were generated using the KAPA RNA HyperPrep™ (Roche) using a polyA selection (Thermo Scientific). Small RNA libraries were prepared using QIAseq™ miRNA Library Kit (Qiagen). Sequencing was performed on the Illumina NextSeq500 system.

Database generation. All RNA-sequencing reads were trimmed by Trimmomatic v0.35 and aligned to the GRCh38.99 index with STARv2.5.1b. Transcript expression was quantified in transcript per million (tpm) using Kallisto v0.43.0 with default parameters.

Canonical proteome. The canonical proteome was built as previously described (13). Sample-specific exomes were built using pyGeno by inserting single-base variants (quality >20) identified with FreeBayes (https://github.com/ekg/freebayes). Annotated open reading frames with tpm >0 were translated in silico from sample-specific exomes, creating the canonical proteome.

ERE proteome. ERE proteomes were built for individual samples as described (23). Ambiguous nucleotides were trimmed from reads of the ERE dataset, followed by a translation in all possible reading frames. Finally, the resulting ERE amino acid sequences were spliced to remove sequences following stop codons. Only sequences of at least eight amino acids were kept and given a unique ID to generate a theoretical ERE proteome. This database was then concatenated to the canonical proteome, generating the personalized ERE proteome used for MAP identification.

Small RNA (smRNA) proteome. smRNA sequencing reads were concatenated in fastq.gz files by the Qiagen software. K-mer databases (24-long) were generated with Jellyfish v2.2.3 and assembled into contigs as described (11). Then, the contigs were 3-frame translated and the different polypeptides were linked with JJ linkers to avoid bias linked to shorter sequences. This database was concatenated with each sample's canonical proteome for MAP identification.

Cancer-specific proteome. Cancer-specific proteomes were assembled using k-mer profiling as described (11,13). k-mers (33-nucleotide-long) present at least once in the mTECs k-mer database were removed from each cancer sample database, and the remaining k-mers were assembled into contigs. Finally, the contigs were 3-frame translated and the different polypeptides were linked with JJ linkers. This database was concatenated with each sample's canonical proteome for MAP identification.

Mass spectrometry analyses. MHC I immunoprecipitation, tandem mass tag (TMT) labeling, and liquid chromatography-MS/MS analyses (LC-MS/MS) were performed as described (24). LC-MS/MS data were searched against the relevant database using PEAKS 10.5 or Peaks X Pro (Bioinformatics Solution Inc.). For peptide identification, tolerance was set at 10 ppm and 0.01 Da for precursor and fragment ions, respectively. Oxidation (M), deamidation, and TMT modification were set as variable modifications. Following peptide identification, the modified target-decoy approach built-in PEAKS was used to apply a sample-specific threshold on the PEAKS scores to ensure a false discovery rate (FDR) of 1%, calculated as the ratio between the number of decoy hits and the number of target hits above the score threshold. PEAKS scores corresponding to a 1% FDR for each sample were determined, and peptides that passed the threshold were further filtered to match the following criteria: peptide length between 8 and 11 amino acids, binding affinity rank to the sample's HLA alleles <2% based on NetMHCpan-4.1b (25). These filtering steps were performed with the use of MAPDP (26).

Genomic origin and validation of MAPs of interest (MOIs). The tumor antigen candidates were identified as previously described (11,13,14). When a MAP aligned to an exonic sequence with ≥1 read, the genomic origin of the MAP was considered exonic. Otherwise, the alignment with the highest number of reads was used to confer the genomic origin of the MOI. All final alignments for TSAs and TAAs were manually validated with Integrative Genomics Viewer (IGV). MOIs for which reads did not match a concordant genomic location or matched hypervariable regions (such as the MHC, Ig, or TCR genes) were excluded.

TSA candidates were classified as mTSAs if they contained variants in their MAP-coding sequences that did not match with known germline polymorphisms [reported in Database of Single-Nucleotide Polymorphisms (dbSNP) v149, http://www.ncbi.nlm.nih.gov/SNP/]. Leucine and isoleucine variants are not distinguishable by standard MS approaches. Hence, MAPs for which an existing variant was flagged were discarded as non-MOI unless it presented a higher RNA expression than the variant or mapped in inadequate regions (in the case of ERE MAP candidates and smRNA MAP candidates). Additionally, all MOI, ERE MAP, and smRNA MAP candidates were validated with the COMET software (27) using the filters used with Peaks and an FDR of 5%. For peptides not re-identified with COMET, manual spectra validation was done in-house to keep further or discard MAPs.

MOI expression in tissues. The expression of the MOI coding sequence was evaluated in normal tissues from GTEX (n=50 samples per tissue), mTECs (n=11), purified blood and bone marrow samples (n=6), and breast cancer tissues from TCGA (n=1109).

Predicted TAA and TSA presentation in TCGA breast cancer samples and survival analyses. A TAA or TSA was considered to be presented in a TCGA sample only when the MAP-coding transcript was expressed (>2 rphm), and the patient had an HLA allotype that could present this MAP according to NetMHCPan4.1 (25). HLA alleles for TCGA patients were determined with the Optitype software (28). TCGA survival data was obtained with the “TCGAbiolinks” package (29). Patients with more than one biopsy were removed from the analyses not to duplicate their contribution to the results, leaving 915 patients. Kaplan-Meier survival curves and log-rank tests were generated with the TCGAbiolinks package.

Single-cell analysis. Raw single-cell data from 14 breast cancer patients published by Qian et al. (30) were obtained through their website: http://scope.lambrechtslab.org/. Visualization graphs were developed with the Seurat package (31).

FEST assays. FEST assays were conducted as previously described (Danilova et al., Cancer Immunol Res. 2018 August; 6(8):888-899. Epub 2018 Jun. 12, 2018), with minor modifications. Five millions T cells were co-cultured during 20 days, with a restimulation at day 10, with autologous T cell-depleted PBMCs pulsed with individual peptides. After in vitro expansion, CD8 T cells were isolated using Human CD8+ T Cell Isolation Kit (Miltenyi). DNA extraction was performed with QIAGEN DNA blood mini kit and was followed by TCR Vβ CDR3 sequencing using the ImmunoSEQ platform. Raw data were processed with the FEST web tool (www.stat-apps.onc.jhmi.edu/FEST). The following parameters were used: FDR 1%, Fold change >5, minimal number of templates of 1 and “Ignore baseline threshold”. The two negative control groups consisted of CD8⁺ T cells co-cultured with unpulsed autologous T-cell depleted PBMCs and uncultured CD8⁺ T cells.

Statistical analyses and data visualization. Analyses and figures were performed using the R v4.0.0. The “gplots” and “ggplot2” packages in R were used to generate the different graphs. Tests involving comparisons of distributions were performed with the t-test or the one-way ANOVA test, as necessary. Differential gene expression analyses were performed with the limma package (32). GSEA analyses were performed with the “fgsea” package (33).

### Example 2: Global Proteogenomic Strategy for MAPs Identification in Primary Breast Cancer Samples

26 primary breast cancer samples (14 HR+ and 12 TNBC) from untreated patients were analyzed. MAPs were identified by MS analyses using a previously described proteogenomic approach (11,13,14). For each sample, personalized reference databases were built by in silico translation of RNA-sequencing data. These databases included four modules: 1) a canonical proteome (in-frame translation of protein-coding exons), 2) an endogenous retroelements (ERE) proteome, 3) a small-RNA proteome, and 4) a cancer-specific proteome. The cancer-specific proteome contained RNA sequencing reads present in the tumor sample but not in a collection of mTECs (FIG. 8).

### Example 3: Canonical Immunopeptidomes of HR+ and TNBC Tumors are Similar

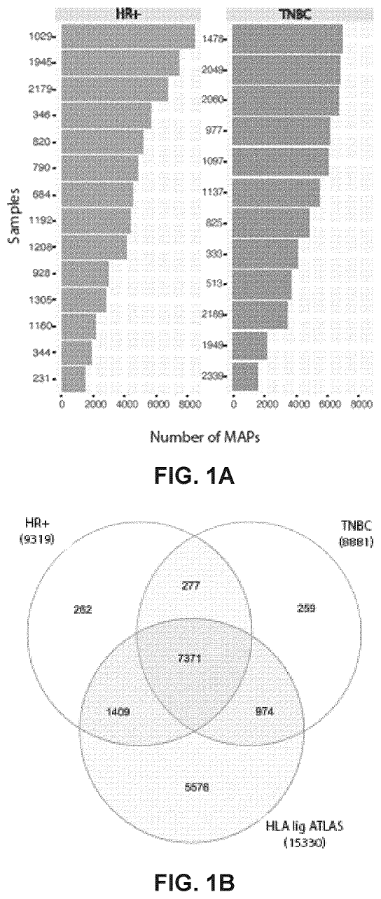

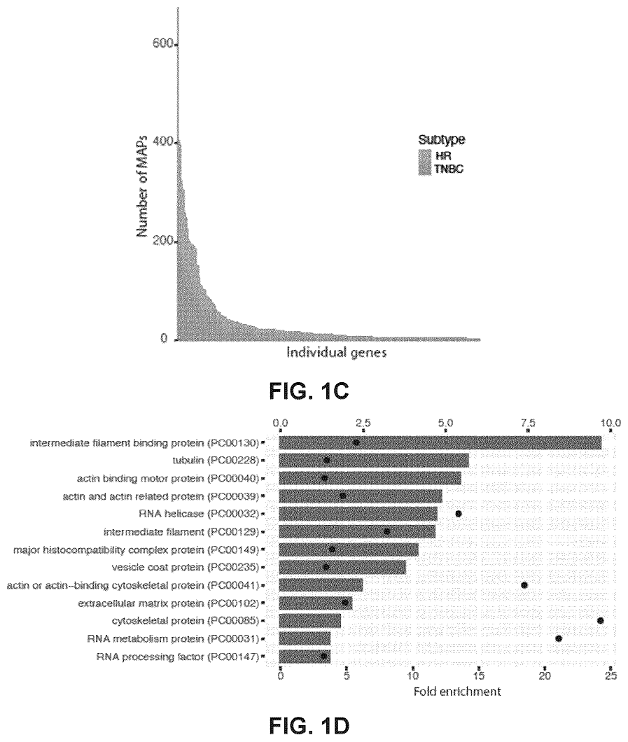

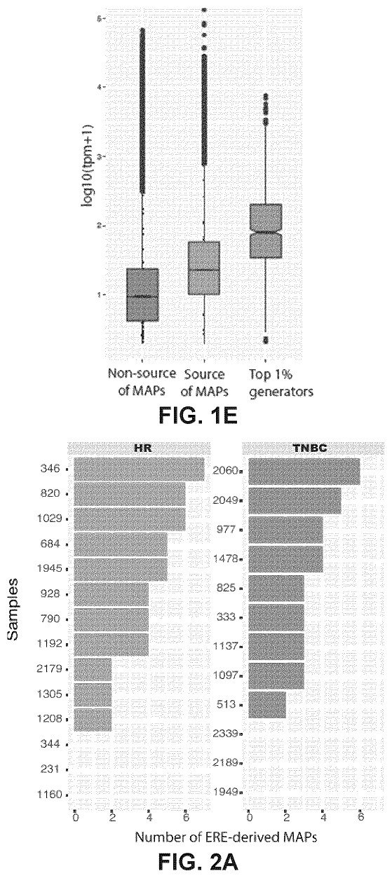

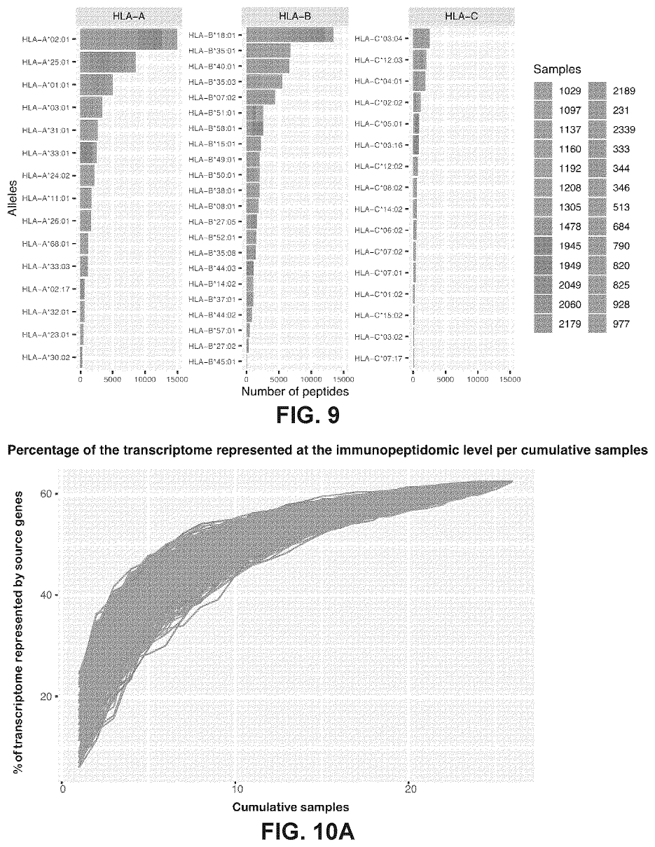

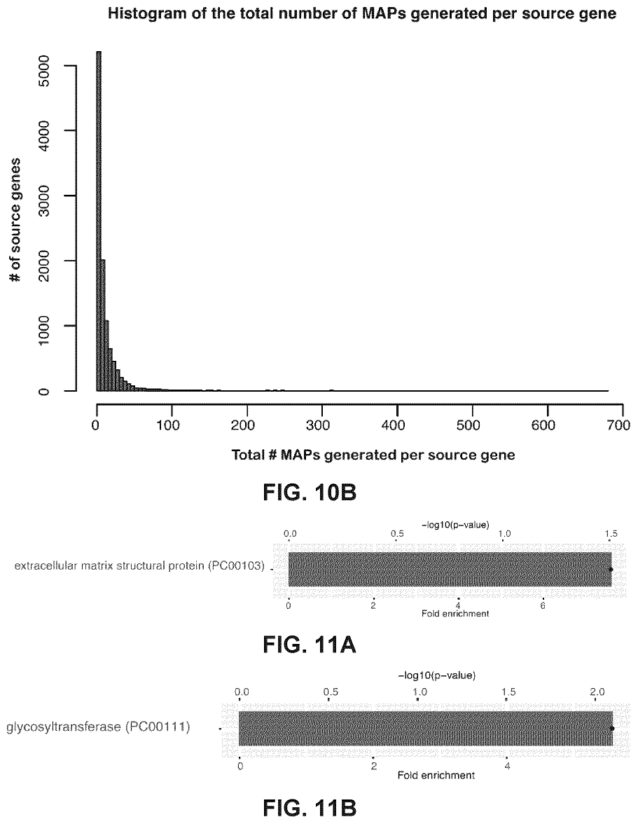

MAPs coded by the canonical reading frame of annotated protein-coding genes are collectively referred to as the canonical immunopeptidome. A total of 57 094 canonical MAPs deriving from 10,552 protein-coding genes were identified. The mean number of MAPs per tumor sample was 4633, with no differences between HR⁺ and TNBC samples (t-test; p>0.05, FIG. 1A). MAPs were presented by 53 different HLA alleles, with >20% presented by two common alleles: HLA-A*02:01 and HLA-B*18:01 (FIG. 9). Consistent with a previous report (34), 62% of the transcriptome, defined as genes with more than one transcript per kilobase million (TPM), generated MAPs (FIG. 10A). A high proportion of genes (72.5%, n=7371) led to MAP generation in both HR+ and TNBC tumors (FIG. 1B), and most of them (92%) were also reported as MAP source genes in normal tissues from the HLA ligand ATLAS (18).

Out of the 798 canonical MAPs not listed in the HLA ligand ATLAS, 277 were found in both HR⁺ and TNBC samples, 259 were unique to TNBC, and 262 to HR⁺ samples (FIG. 1B). The 277 canonical MAPs shared by HR⁺ and TNBC samples were coded by a gene set enriched in extracellular matrix protein-coding genes (fold enrichment 7.59, p<0.05) (FIG. 11A). The 259 canonical MAPs found only in TNBC samples showed enrichment in glycosyltransferase protein class (fold enrichment 5.58, p<0.05) (FIG. 11B). No particular enrichment was found for the 262 MAPs found only in HR⁺ samples.

Source genes did not uniformly contribute to the immunopeptidome. While 49% of the source genes from the dataset generated ≤5 MAPs, 51% generated >5 MAPs, and a sizeable proportion of them coded for >100 MAPs MAPs (FIG. 1C and FIG. 10B). The top 1% generators of MAPs were notably enriched in cytoskeletal and extracellular matrix protein classes (FIG. 1D). Altogether, these results reveal a substantial overlap between the canonical immunopeptidome overlap of HR⁺ and TNBC samples and a conspicuous enrichment in MAPs derived from cytoskeletal and extracellular matrix proteins. The immunopeptidome projects at the cell surface a representation of proteins actively translated and degraded into the cells (35). The overrepresentation of MAPs derived from cytoskeletal and extracellular matrix proteins is, therefore, coherent with the crucial role of extracellular matrix remodeling in breast cancer tumorigenesis (36-38).

### Example 4: The Contribution of EREs and smRNAs to the Non-Canonical Breast Cancer Immunopeptidome

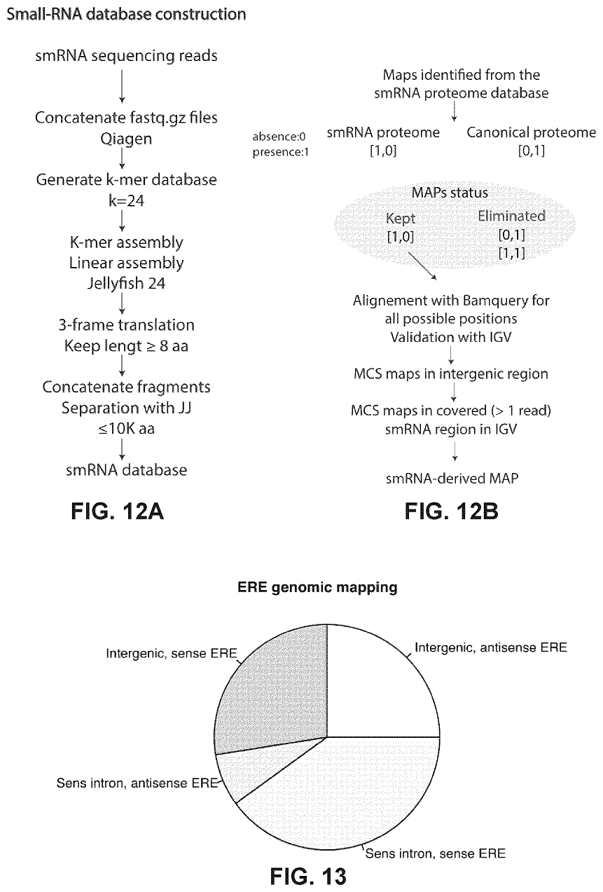

EREs and smRNAs are implicated at different steps in neoplastic transformation, including in breast cancer (39,40). Furthermore, EREs have already been shown to code for immunogenic MAPs in mice and humans (13,23). Therefore, the personalized proteogenomic databases for the presence of ERE- and smRNA-coded MAPs in breast cancer was specifically interrogated. ERE-coding transcripts were retrieved from bulk RNA sequencing, whereas smRNA sequencing was used to build smRNA databases (FIG. 12A-B).

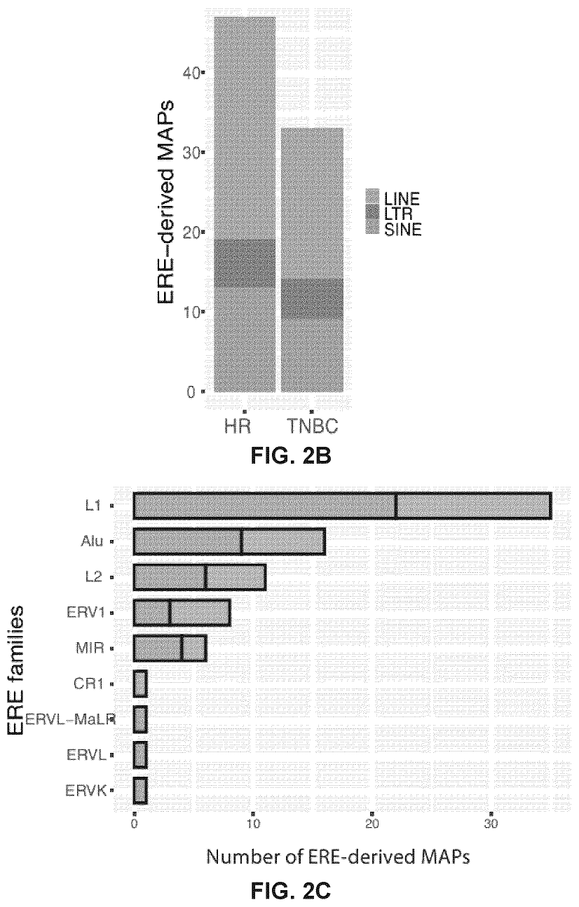

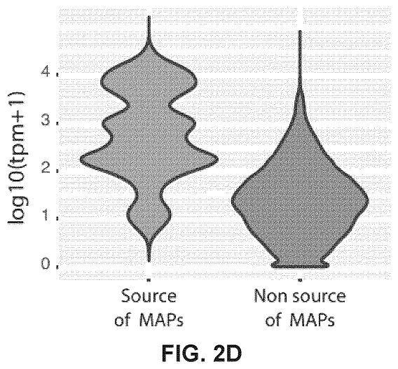

75 ERE-derived MAPs, equally distributed between HR⁺ and TNBC samples (FIG. 2A, Table 1), were identified. ERE-derived MAPs mapped similarly to intronic and intergenic regions (FIG. 13). The three main classes of EREs contributed similarly to the immunopeptidome of HR⁺ and TNBC samples (FIG. 21B). EREs that generated MAPs were expressed at a higher level than EREs generating no MAPs (FIG. 20). Only 9/22 ERE families led to MAP generation, and the L1 family was the most important contributor (FIG. 2C). Only three smRNA-derived MAPs (Table 2) were identified: one derived from a piRNA and two others from snRNAs. It was concluded that EREs but not smRNA generate a significant number of MAPs in breast cancer tumors. However, ERE MAPs can also be found in normal tissues (23). Therefore, further analyses had to be conducted to evaluate whether ERE MAPs could be labeled as TAAs or TSAs.

TABLE 1
ERE-derived MAPs

| Peptide sequence | ERE | ERE | ERE | | |
|---|---|---|---|---|---|
| (SEQ ID NO) | class | family | name | Samples | Allele |
| AETLSPLSL (62) | LINE | L2 | L2-3_Crp | 977 | HLA-B*40:01 |
| AEVERSPEP (63) | SINE | Alu | AluJb | 1478, 1305 | HLA-B*50:01 |
| ALSSVPLIY (64) | LINE | L1 | L1MC2 | 820 | HLA-B*15:01 |
| AQLHIPVV (65) | SINE | Alu | AluJb | 346, 928 | HLA-B*52:01 |
| DGSHALVL (66) | LTR | ERV1 | LTR28C | 1192 | HLA-B*51:01 |
| DLSPGVQGY (67) | SINE | Alu | FRAM | 1478 | HLA-A*25:01 |
| DVSGVSSFW (68) | LTR | ERV1 | LTR12/C | 1137, 206 | HLA-A*25:01 |
| EAGSLHLQR (69) | LINE | CR1 | L3 | 1097 | HLA-A*68:01 |
| ELISELSKI (70) | LINE | L1 | L1ME3A | 513 | HLA-A*26:01 |
| ETQIKTTTR (71) | LINE | L1 | L1PA3 | 684 | HLA-A*26:01 |
| EVERSPEPR (72) | SINE | Alu | AluJb | 1097 | HLA-A*68:01 |
| EVRGGGITR (73) | SINE | Alu | AluSx3 | 333 | HLA-A*33:03 |
| EWVGLLESR (74) | SINE | Alu | AluJb | 1945 | HLA-A*33:01 |
| FEFLFTNL (75) | LINE | L1 | L1PBa1 | 2049 | HLA-B*18:01 |
| FPHSLLSVL (76) | LINE | L2 | L2c | 1192 | HLA-B*35:03 |
| GEDLEKVTI (77) | LINE | L2 | L2a | 333 | HLA-B*40:01 |
| GEKEILITV (78) | LINE | L1 | L1MA6 | 684 | HLA-B*49:01 |
| GIRPPTSVR (79) | SINE | MIR | MIR | 825 | HLA-A*31:01 |
| GQIKKLLQM (80) | LTR | ERVK | HERVK9-int | 820 | HLA-B*15:01 |
| IDSISWLL (81) | LINE | L1 | L1MA8 | 2060 | HLA-B*37:01 |
| IDSLSWLL (82) | LINE | L1 | L1MA5 | 2060 | HLA-B*37:01 |
| IEMNSILFQM (83) | LINE | L1 | L1MC4a | 2060 | HLA-B*18:01 |
| IISSNILSV (84) | LINE | L1 | L1MB7 | 513 | HLA-A*02:01 |
| ILLPQPPGV (85) | SINE | Alu | AluSz | 2179 | HLA-A*02:01 |
| ILTEQEEIL (86) | SINE | MIR | MIRc | 820 | HLA-A*02:01 |
| IPLLSQIY (87) | LINE | L2 | L2c | 1097 | HLA-B*35:01 |
| ISAISALW (88) | SINE | Alu | FLAM_C | 1945 | HLA-B*58:01 |
| ISLLPISEK (89) | LINE | L2 | L2 | 684 | HLA-A*03:01 |
| ISSAFTLFL (90) | LINE | L2 | L2b | 2049 | HLA-C*12:03 |
| ITATSASW (91) | SINE | Alu | AluSq2 | 1945 | HLA-B*58:01 |
| IYLIPEPVLF (6) | SINE | MIR | MIRb | 1029 | HLA-A*24:02 |
| IYLVEPLKF (92) | LINE | L1 | L1ME3G | 1029 | HLA-A*24:02 |
| LESIGYVL (93) | LINE | L1 | L1ME3E | 825 | HLA-B*18:01 |
| LISSNILSV (94) | LINE | L1 | L1MC5 | 820, 1945 | HLA-A*02:01 |
| LLNSQWVSK (95) | LINE | L1 | L1MA8 | 1137 | HLA-A*03:01 |
| LPLLVNII (96) | LINE | L1 | L1MB3 | 1192 | HLA-B*51:01 |
| LPSPSIVSL (97) | LINE | L2 | L2c | 1029 | HLA-B*35:01 |
| LRNLHNVL (98) | LINE | L1 | L1MA3 | 1208 | HLA-B*14:02 |
| LTSGWLLSK (99) | LINE | L1 | L1MA5A | 684 | HLA-A*03:01 |
| NEALAAIL (100) | LTR | ERVL- | MLT1F | 346 | HLA-B*18:01 |
| | | MaLR | | | |
| NETAAYKL (101) | LINE | L1 | L1MA9 | 2049 | HLA-B*18:01 |
| NQTPHILI (102) | LINE | L1 | L1PA7 | 1208 | HLA-B*14:02 |
| QDSLVLNL (103) | SINE | MIR | MIR | 2060 | HLA-B*37:01 |
| QPAAASSL (104) | LTR | ERV1 | LTR12_ | 790 | HLA-B*07:02 |
| QQQKLLKI (105) | LINE | L1 | HAL1 | 928 | HLA-B*52:01 |
| RFIDLTSTF (106) | LTR | ERVL | HERVL-int | 820 | HLA-B*15:01 |
| RLLPLEVLL (107) | LINE | L2 | L2b | 1478 | HLA-C*04:01 |
| RSFTMRSSR (108) | LINE | L1 | L1MA6 | 346, 1192 | HLA-A*31:01 |
| RSMRVGVTR (8) | LINE | L1 | L1PA2 | 825 | HLA-A*31:01 |
| RTLAVSVTALK (109) | LTR | ERV1 | LTR12 | 1137 | HLA-A*03:01 |
| SIMHSFILK (110) | LINE | L1 | L1M6 | 1305 | HLA-A*03:01 |
| SLFNLFPR (111) | LINE | L2 | L2c | 346 | HLA-A*31:01 |
| SPAEKQII (112) | LINE | L1 | HAL1b | 790 | HLA-B*07:02 |
| SPWIILRDI (113) | LINE | L1 | L1M1 | 790 | HLA-B*07:02 |
| SQLISLQTL (114) | LINE | L1 | L1M4 | 820 | HLA-B*38:01 |
| SSDPPTSTL (115) | SINE | Alu | AluJb | 977 | HLA-C*03:04 |
| SSYLVSQL (116) | LINE | L2 | L2 | 928 | HLA-B*52:01 |
| SVVSLWVQK (117) | LINE | L1 | L1MA8 | 346 | HLA-A*11:01 |
| TESGINVTL (118) | LINE | L2 | L2 | 1029 | HLA-B*40:01 |
| TILESSLTL (119) | LINE | L1 | L1ME3E | 977 | HLA-C*03:04 |
| TLFNFLVSL (120) | LTR | ERV1 | MER34B-int | 2049 | HLA-A*02:01 |
| TLNDLTTSW (121) | LINE | L1 | HAL1ME | 1478 | HLA-A*25:01 |
| TLSGWLLSK (122) | LINE | L1 | L1MB3 | 790 | HLA-A*03:01 |
| TMLKLVLI (123) | SINE | MIR | MIR | 346 | HLA-B*52:01 |
| TPRHLIVRF (124) | LINE | L1 | L1PA8 | 2179 | HLA-B*07:02 |
| TTNPSIKKR (125) | LTR | ERV1 | LTR12F | 333 | HLA-A*33:03 |
| VDNSTISI (126) | LINE | L1 | L1MC1 | 2060 | HLA-B*37:01 |
| VEVDRLLEV (127) | SINE | Alu | AluSx1 | 684 | HLA-B*49:01 |
| VGFILPSV (128) | SINE | MIR | MIR3 | 346 | HLA-B*52:01 |
| VQFIIYQL (129) | LINE | L1 | L1ME1 | 928 | HLA-B*52:01 |
| VRSSIILTY (130) | LINE | L1 | L1MEf | 1945 | HLA-C*03:16 |
| VSAQAGVQW (131) | SINE | Alu | AluSx | 2049 | HLA-A*25:01 |
| VYLSSLALL (132) | LTR | ERV1 | HERVIP10F- | 1029 | HLA-A*24:02 |
| | | | int | | |
| WLMPVLPAL (133) | SINE | Alu | AluJr | 977 | HLA-A*02:01 |
| YVAQAGLEI (134) | SINE | Alu | AluJo | 1029 | HLA-C*03:04 |

TABLE 2
smRNA-derived MAPs

| Peptide sequence | | Samples for | |
|---|---|---|---|
| (SEQ ID NO) | Origin | identification | Allele |
| ITKVVFPGR (135) | snRNA | 825,346,1192 | HLA-A*31:01 |
| DEDLFTDLY (136) | piRNA | 825 | HLA-B*18:01 |
| WETRLHNLW (137) | snRNA | 1945 | HLA-B*44:03 |

### Example 5: Identification of Potential Therapeutic Targets: TAAs, aeTSAs, and mTSAs

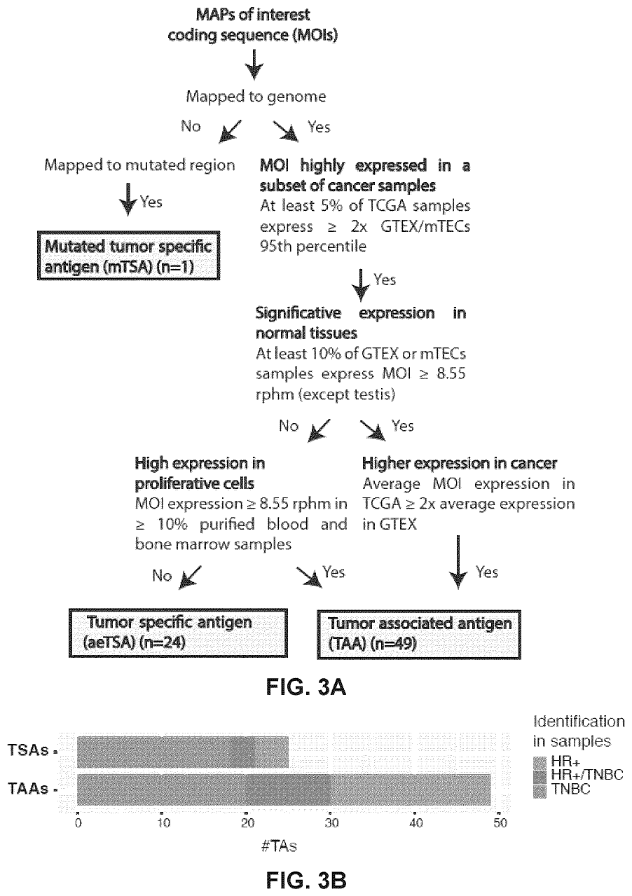

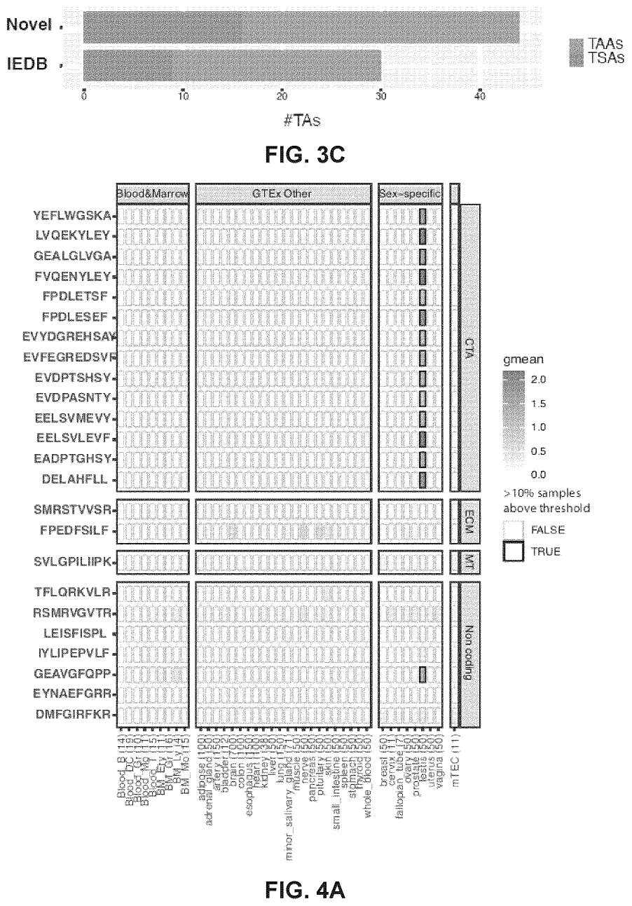

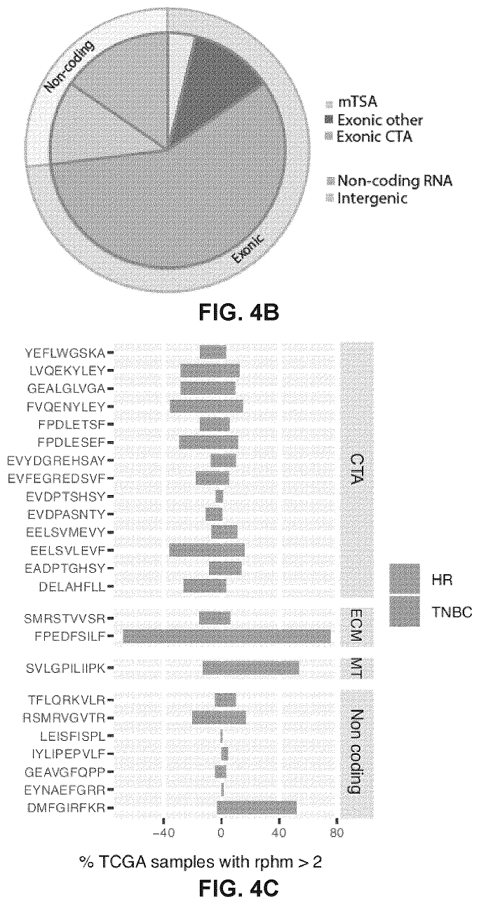

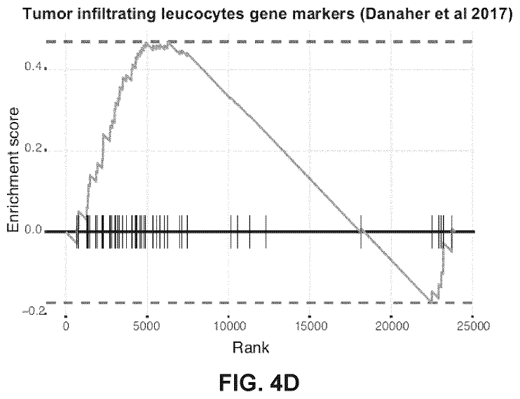

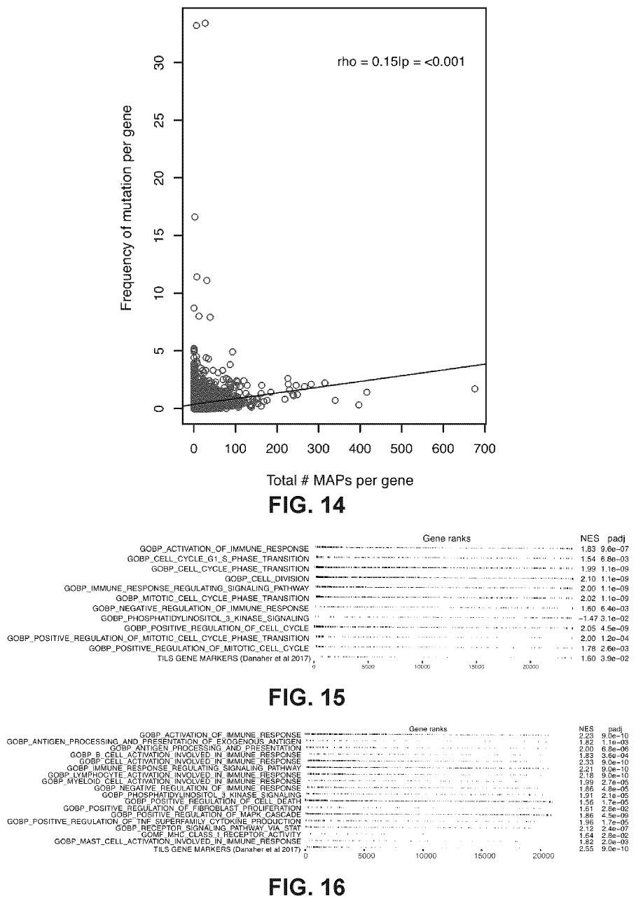

MOIs were identified and classified as TAAs, aeTSAs, or mTSAs with the workflow outlined in FIG. 3A. Overall, 25 TSAs were identified: 1 mTSA and 24 aeTSAs (FIG. 4A). The sole mTSA originated from a non-synonymous mutation in the deubiquitinase OTUB1 gene. This rare mutation is not listed in the COSMIC database (https://cancer.sanger.ac.uk/cosmic). The scarcity of mTSAs led us to ask whether genes frequently mutated in breast cancer were represented in the immunopeptidome. A slightly positive correlation was found between the frequency of mutations identified by the TCGA consortium and the generation of MAPs in the dataset (p<0.001) (FIG. 14). This means that there is no negative bias against the representation of highly mutated genes in the breast cancer immunopeptidome. Hence, highly mutated genes generate MAPs, but these MAPs do not derive from the mutated region. This is consistent with the fact that MAPs preferentially originate from particular regions of MAP source proteins (MAP “hotspots”) (18,34,41). The most parsimonious explanation for the scarcity of mTSAs is that breast cancers harbor relatively few mutations, and these mutations are not located in MAP hotspots.

In the studies of TAAs and aeTSAs, only MAPs coded by transcripts expressed in at least 5% of TCGA breast cancer cohort samples were considered. It was assumed that antigens expressed in fewer samples had little interest. Classification of unmutated MAPs as TAAs or aeTSAs was based on comprehensive transcriptomic analyses of their expression in i) breast cancer samples from the TCGA (n=1109), ii) 50 normal tissues from GTEX (@ 50 samples per tissue), iii) mTECs (n=11), iv) purified blood and bone marrow samples (n=6). Blood and marrow cells are used as a surrogate for rapidly proliferating cells since 90% of cells produced daily in humans are hematopoietic cells (42). It must be stressed that the expression profiling only considers the MAP-coding sequence, not the entire gene or genomic region. Thus, aberrations in RNA splicing commonly lead to the presence of protein isoforms only in cancer cells. MAPs derived from such a cancer-specific isoform are labeled as aeTSAs even if other isoforms are expressed in normal cells.

MAPs whose expression was below 8.55 reads per hundred million (rphm) in all normal tissues except the testis were labeled as aeTSAs. As reported (11), 8.55 rphm was used as a threshold because expression below this level is associated with a very low probability of MAP generation. Otherwise, MOIs with above threshold expression in one or more normal tissues were labeled as TAAs if overexpressed in neoplastic or highly proliferative tissues compared to normal non-hematopoietic tissues. This strategy led to the identification of 24 aeTSAs and 49 TAAs, most of which are novel since they are not listed in the Immune Epitope Database (IEDB) (FIG. 3B-C, Tables 3 and 4).

TABLE 3
TSAs identified in the present study

| Peptide | | | | | | Linked to |
|---|---|---|---|---|---|---|
| sequence | | | Genomic | | Present | breast |
| (SEQ ID NO) | Category | Gene | origin | Allele | in IEDB | cancer |
| SVADLLSSF (10) | mTSA | OTUB1 | Exon | HLA-A*25:01 | No | No |
| DELAHFLL (56) | aeTSA | MAGEA4 | Exon | HLA-B*18:01 | No | No |
| DMFGIRFKR (2) | aeTSA | NA | ncRNA | HLA-A*33:01 | No | No |
| EADPTGHSY | aeTSA | MAGEA1 | Exon | HLA-B*35:08, | Yes | No |
| (36) | | | | HLA-B*35:01 | | |
| EELSVLEVF | aeTSA | MAGEA6 | Exon | HLA-B*18:01 | No | No |
| (58) | | | | | | |
| EELSVMEVY | aeTSA | MAGEA1 | Exon | HLA-B*18:01 | No | No |
| (59) | | | | | | |
| EVDPASNTY | aeTSA | MAGEA4 | Exon | HLA-A*01:01 | Yes | No |
| (37) | | | | | | |
| EVDPTSHSY | aeTSA | MAGEA11 | Exon | HLA-A*01:01 | Yes | No |
| (38) | | | | | | |
| EVFEGREDSVF | aeTSA | MAGEA2B | Exon | HLA-A*25:01 | Yes | Yes |
| (138) | | | | | | |
| EVYDGREHSAY | aeTSA | MAGEA1 | Exon | HLA-A*25:01 | Yes | No |
| (39) | | | | | | |
| EYNAEFGRR | aeTSA | NA | Intergenic | HLA-A*33:01 | No | No |
| (3) | | | | | | |
| FPDLESEF (60) | aeTSA | MAGEA6 | Exon | HLA-B*35:08 | No | No |
| FPDLETSF (57) | aeTSA | MAGEA12 | Exon | HLA-B*35:08 | No | No |
| FPEDFSILF (4) | aeTSA | COL11A1 | Exon | HLA-B*35:01 | No | No |
| FVQENYLEY | aeTSA | MAGEA6 | Exon | HLA-A*25:01, | Yes | No |
| (40) | | | | HLA-B*35:01, | | |
| | | | | HLA-B*35:08 | | |
| GEALGLVGA | aeTSA | MAGEA12 | Exon | HLA-B*50:01 | Yes | No |
| (41) | | | | | | |
| GEAVGFQPP | aeTSA | LINC00221 | ncRNA | HLA-B*50:01 | No | No |
| (5) | | | | | | |
| IYLIPEPVLF (6) | aeTSA | NA | Intergenic | HLA-A*24:02 | No | No |
| LEISFISPL (7) | aeTSA | NA | Intergenic | HLA-B*50:01 | No | No |
| LVQEKYLEY | aeTSA | MAGEA1 | Exon | HLA-A*01:01 | Yes | No |
| (42) | | | | | | |
| RSMRVGVTR | aeTSA | NA | Intergenic | HLA-A*31:01 | No | No |
| (8) | | | | | | |
| SMRSTWVSR | aeTSA | ITIH6 | Exon | HLA-A*31:01 | No | No |
| (9) | | | | | | |
| SVLGPILIIPK (1) | aeTSA | ABCC11 | Exon | HLA-A*11:01 | No | No |
| TFLQRKVLR | aeTSA | LINC00578 | ncRNA | HLA-A*31:01 | Yes | No |
| (43) | | | | | | |
| YEFLWGSKA | aeTSA | MAGEA9 | Exon | HLA-B*50:01 | No | No |
| (61) | | | | | | |

TABLE 4
TAAs identified in the present study

| | | | | | | Linked to |
|---|---|---|---|---|---|---|
| Peptide | | | Genomic | | Present | breast |
| sequence | Category | Gene | origin | Allele | in IEDB | cancer |
| AETDITETSI | TAA | COL11A1 | Exon | HLA-B*49:01 | No | No |
| (11) | | | | | | |
| DEFNGVPSY | TAA | SKA1 | Exon | HLA-B*18:01 | Yes | Yes |
| (139) | | | | | | |
| DEQTPIHISW | TAA | CDKN3 | Exon | HLA-B*44:03 | Yes | Yes |
| (140) | | | | | | |
| DETLTALQF | TAA | CENPE | Exon | HLA-B*18:01 | Yes | No |
| (44) | | | | | | |
| DIIEYDYEY (12) | TAA | COL11A1 | Exon | HLA-B*35:01, | No | No |
| | | | | HLA-A*26:01 | | |
| DVITIIDRSF | TAA | MKI67 | Exon | HLA-A*25:01 | Yes | Yes |
| (141) | | | | | | |
| EEIFTEEY (13) | TAA | COL11A1 | Exon | HLA-B*18:01 | No | No |
| EEQYIAQF (14) | TAA | PRAME | Exon | HLA-B*18:01 | No | No |
| EEYLVPQAF | TAA | ERBB4 | Exon | HLA-B*18:01 | Yes | Yes |
| (142) | | | | | | |
| ELFQHVSLM | TAA | CYP4Z1 | Exon | HLA-A*26:01 | No | No |
| (15) | | | | | | |
| EVFEGDIQQF | TAA | COL11A1 | Exon | HLA-A*25:01, | Yes | No |
| (45) | | | | HLA-A*26:01 | | |
| EVKSITKER (46) | TAA | CENPE | Exon | HLA-A*33:03 | Yes | No |
| FAFEKLIQF (47) | TAA | ABCC11 | Exon | HLA-C*12:03 | Yes | No |
| FPDVPDKENF | TAA | KNL1 | Exon | HLA-B*35:08 | Yes | Yes |
| (143) | | | | | | |
| GAYDKARYL | TAA | ABCC11 | Exon | HLA-C*12:03 | No | No |
| (16) | | | | | | |
| GSLGHLQNR | TAA | TTK | Exon | HLA-A*31:01 | Yes | Yes |
| (144) | | | | | | |
| GTHVWVGLYK | TAA | COL10A1 | Exon | HLA-A*11:01 | No | No |
| (17) | | | | | | |
| HHNDLVFKF | TAA | CYP4Z1 | Exon | HLA-B*38:01 | Yes | No |
| (48) | | | | | | |
| HQFTEKVI (18) | TAA | CYP4Z1 | Exon | HLA-B*52:01 | No | No |
| ITDDQLLAL (49) | TAA | PRAME | Exon | HLA-C*08:02 | Yes | No |
| KLKQDTTQK | TAA | CYP4Z1 | Exon | HLA-A*03:01 | No | No |
| (19) | | | | | | |
| LEIENLNLA (50) | TAA | CENPE | Exon | HLA-B*50:01 | Yes | No |
| LELFQHVSL | TAA | CYP4Z1 | Exon | HLA-B*40:01 | No | No |
| (20) | | | | | | |
| MAVNIVLQL | TAA | ABCC11 | Exon | HLA-C*12:03 | No | No |
| (21) | | | | | | |
| MLWKLVENV | TAA | TFAP2B | Exon | HLA-A*02:01 | No | No |
| (22) | | | | | | |
| MPLKHYLLL | TAA | LRCC15 | Exon | HLA-B*35:03 | Yes | No |
| (51) | | | | | | |
| MVFSKLVLR | TAA | COL11A1 | Exon | HLA-A*31:01, | Yes | No |
| (52) | | | | HLA-A*68:01 | | |
| QAWGAGLAY | TAA | LRRC15 | Exon | HLA-B*35:01 | No | No |
| (23) | | | | | | |
| QLLALLPSL (53) | TAA | PRAME | Exon | HLA-A*02:01 | Yes | No |
| SEADLQAEV | TAA | CYP4Z1 | Exon | HLA-B*49:01 | No | No |
| (24) | | | | | | |
| SLPDNVFSNL | TAA | LRRC15 | Exon | HLA-C*01:02 | No | No |
| (25) | | | | | | |
| SQGQIFSKF | TAA | CYP4Z1 | Exon | HLA-B*15:01 | No | No |
| (26) | | | | | | |
| SRLELFQHV | TAA | CYP4Z1 | Exon | HLA-B*27:05 | No | No |
| (27) | | | | | | |
| SSQGQIFSK | TAA | CYP4Z1 | Exon | HLA-A*11:01 | No | No |
| (28) | | | | | | |
| SVKERESER | TAA | CENPF | Exon | HLA-A*31:01 | No | No |
| (29) | | | | | | |
| SYLKAVFNL | TAA | CYP4Z1 | Exon | HLA-A*24:02 | No | No |
| (145) | | | | | | |
| TLDSYLKAV | TAA | CYP4Z1 | Exon | HLA-A*02:01 | No | No |
| (146) | | | | | | |
| TLFDYEVRL | TAA | UHRF1 | Exon | HLA-A*02:01 | Yes | Yes |
| (147) | | | | | | |
| TQFFIPYTI (148) | TAA | COL10A1 | Exon | HLA-B*52:01 | No | No |
| TTLENLPQK | TAA | COL11A1 | Exon | HLA-A*11:01 | Yes | No |
| (54) | | | | | | |
| VEAPHLPSF | TAA | COL11A1 | 5UTR | HLA-B*18:01 | Yes | No |
| (55) | | | | | | |
| VEEIFTEEY (30) | TAA | COL11A1 | Exon | HLA-B*18:01 | No | No |
| VHDPDYAKILL | TAA | CYP4Z1 | Exon | HLA-B*38:01 | No | No |
| (31) | | | | | | |
| VPWEDKISI (32) | TAA | CST5 | Exon | HLA-B*51:01 | No | No |
| VTTLALTF (33) | TAA | COL11A1 | Exon | HLA-B*58:01 | No | No |
| WPMHTPLL (34) | TAA | CST5 | Exon | HLA-B*51:01 | No | No |
| WPMHTPLLL | TAA | CST5 | Exon | HLA-B*35:03 | No | No |
| (35) | | | | | | |
| YEDLSQKY | TAA | CENPF | Exon | HLA-B*18:01 | Yes | Yes |
| (149) | | | | | | |
| YPVKEFEVY | TAA | CYP4Z1 | Exon | HLA-B*35:01 | Yes | Yes |
| (150) | | | | | | |

### Example 6: TSAs are More Abundant in TNBC than HR⁺ Breast Cancers

Of the 24 aeTSAs, 17 were coded by canonical exons: 14 belonged to the MAGE family of CTAs, two to genes coding extracellular matrix components (COL11A1, ITH6), and one to a transmembrane protein-coding gene (ABCC11) (FIG. 4 and Table 3). Seven aeTSAs were derived from non-protein-coding regions, two of which overlapped EREs and can be classified as ERE-derived MAPs (FIG. 4 and Table 3).

Most of the aeTSAs were found in TNBC samples (FIG. 3B). It was next assessed whether this enriched identification in TNBC samples correlated with TSA expression in the TOGA cohort. The proportion of tumors expressing individual aeTSAs of the CTA class was superior in TNBC relative to HR₊ tumors (19% vs. 8%, p=0.004) (FIG. 4C). There was no significant difference in the distribution for the other categories of TSAs.

It was next evaluated whether aeTSA presentation would correlate with immune infiltration. An aeTSA was considered to be presented in a TCGA sample only when the MAP-coding transcript was expressed, and the patient had an HLA allotype that could present this MAP (11). TCGA samples were then categorized into two groups presenting high (above median) vs. low (below median) numbers of aeTSAs. Then, a differential gene expression analysis between the two groups was performed. A gene set enrichment analysis using gene markers of leukocytic infiltration described by Danaher et al. (43) showed enrichment of these genes in the group of tumors with high TSA numbers. This suggests that at least some TSAs are immunogenic in vivo. Additionally, it was evaluated if oncogenic pathways involved in immune escape (44) were enriched in samples with high or low levels of TSAs. To this end, the corresponding hallmark gene sets from the Molecular Signature Database (45) was selected, and enrichment in the PI3K pathway was found (NES=−1.48, p<0.05; FIG. 15) in tumors with low numbers of predicted TSAs. The PI3K pathway is implicated in the tumorigenesis, progression, and resistance to treatment in breast cancers (46).

### Example 7: TAAs are Highly Shared in Breast Cancer

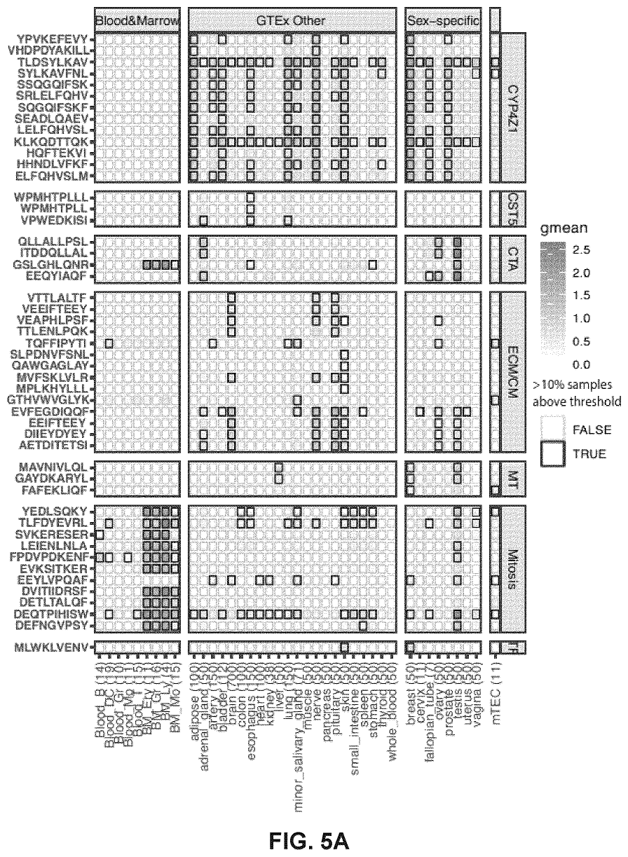

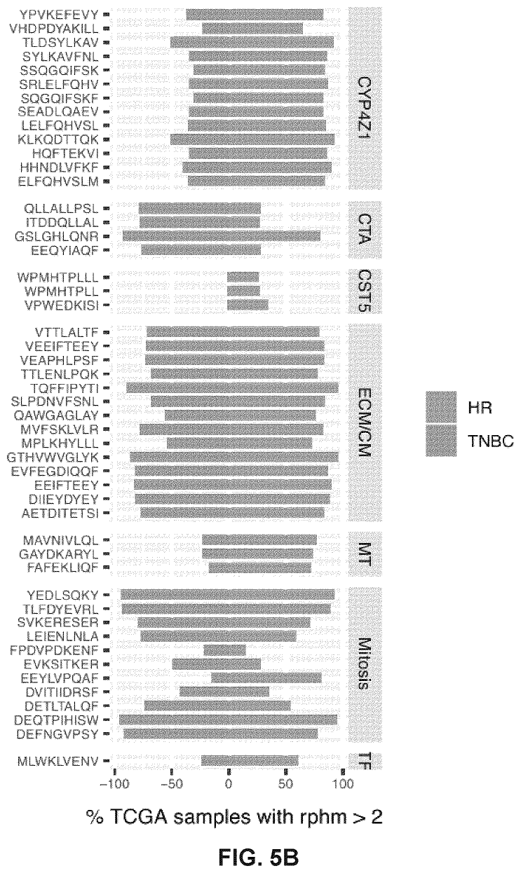

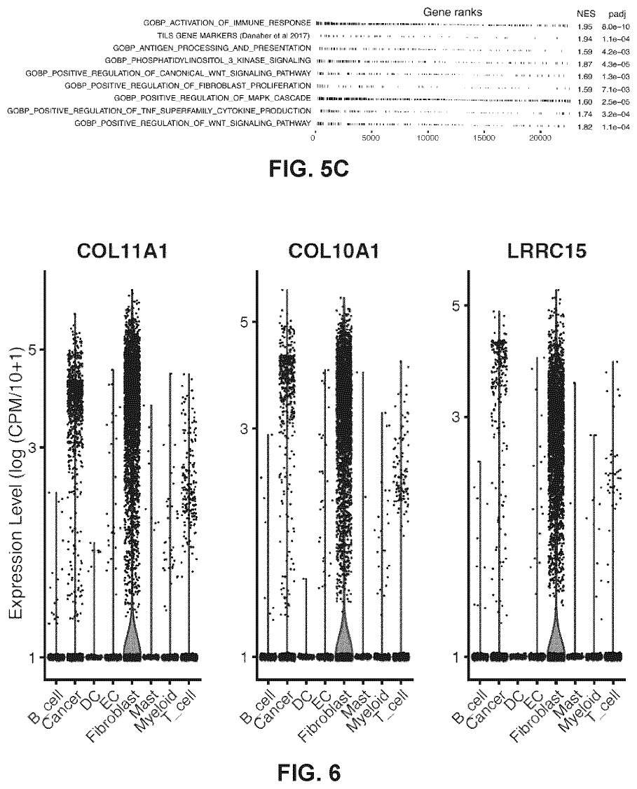

49 TAAs were identified, of which 48 originated from canonical protein-coding regions (FIG. 5A and Table 4). These antigens were highly shared in both HR⁺ and TNBC tumors (FIG. 5B). The largest group of TAAs (n=14) derived from genes (COL11A1, COL10A1, LRRC15) reported as markers of cancer-associated fibroblasts (47). These genes are implicated in extracellular matrix production and cell migration. To confirm their most likely cell of origin, their expression level was evaluated in a single cell dataset from Qian et al. (30), comprising 14 breast cancer tumors (FIG. 6A). All three genes had significantly higher expression in tumor fibroblasts than in other cell subsets, including cancer cells (FIG. 6B; ANOVA; p<0.05). Two other large groups of TAAs were identified. Thirteen TAAs derived from CYP4Z1, which is implicated in many cancer types and elicits autoantibodies against CYP4Z1 in breast cancer patients (48). Eleven TAAs were associated with cell proliferation (FIG. 5B); they are expressed at low levels in mature epithelial and blood cells but at higher levels in bone marrow progenitor cells (FIG. 5A).

The number of TAAs per tumor in the TCGA dataset was predicted using the same criteria as for aeTSA: expression of the MAP-coding sequence and presence of a relevant HLA allotype (FIG. 5C, FIG. 16). HR⁺ and TNBC tumors with a high level of predicted TAAs showed enrichment in immune activation and immunosuppressive pathways, namely the PI3K/mTOR, Wnt/B-catenin, and MAPK pathways. In addition, tumors with numerous TAAs showed an enrichment in markers of fibroblast proliferation. These findings suggest that, in the presence of multiple TAAs, anti-tumor immune responses are mitigated by the activation of immunosuppressive pathways and the accumulation of cancer-associated fibroblasts.

### Example 8: Presentation of Numerous TSAs Improves Overall Survival in TNBC

It was next assessed whether the number of presented TAAs and aeTSAs correlated with the overall survival of TCGA patients. As in the previous example, it was considered that an antigen was presented in a tumor when both the MAP-coding transcript and an appropriate HLA allotype were expressed (11). Patients were divided into two categories: high number of presented antigens (first quartile) and low number of presented antigens (2nd-4th quartiles).

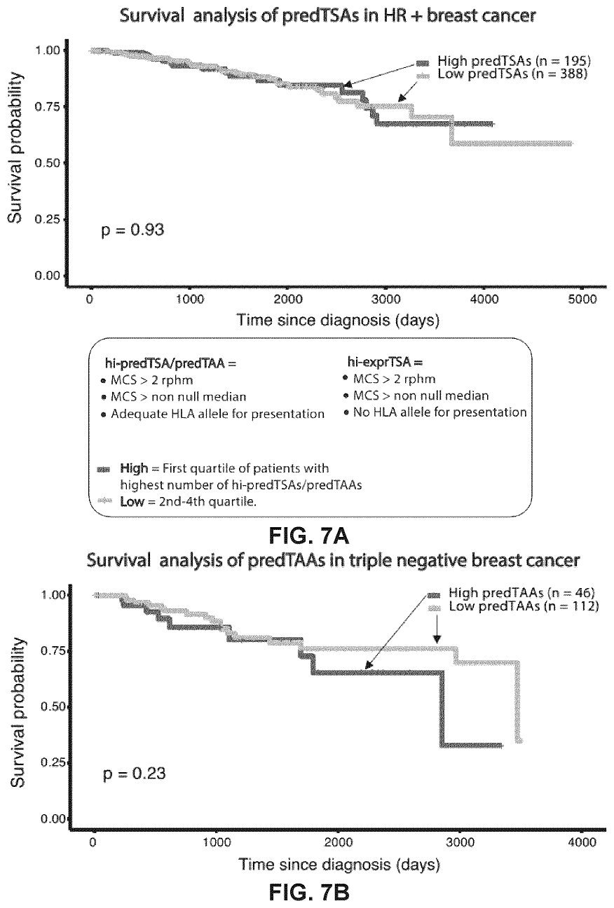

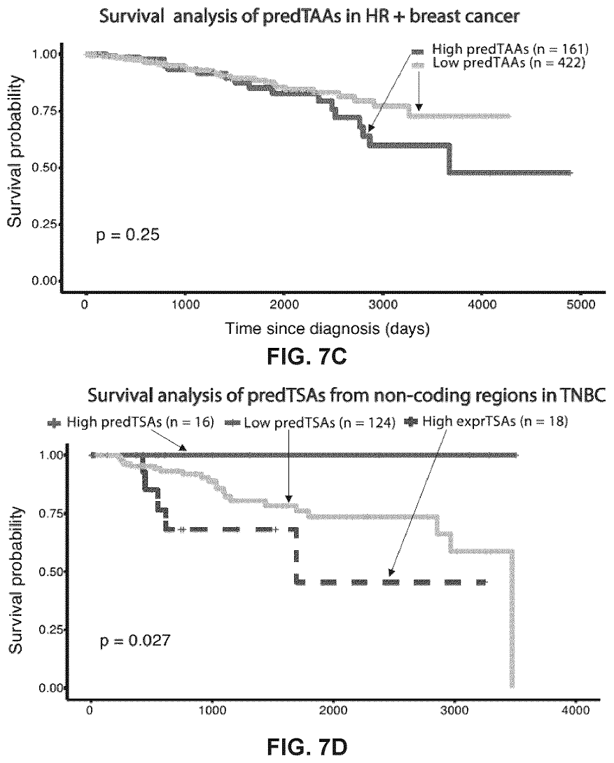

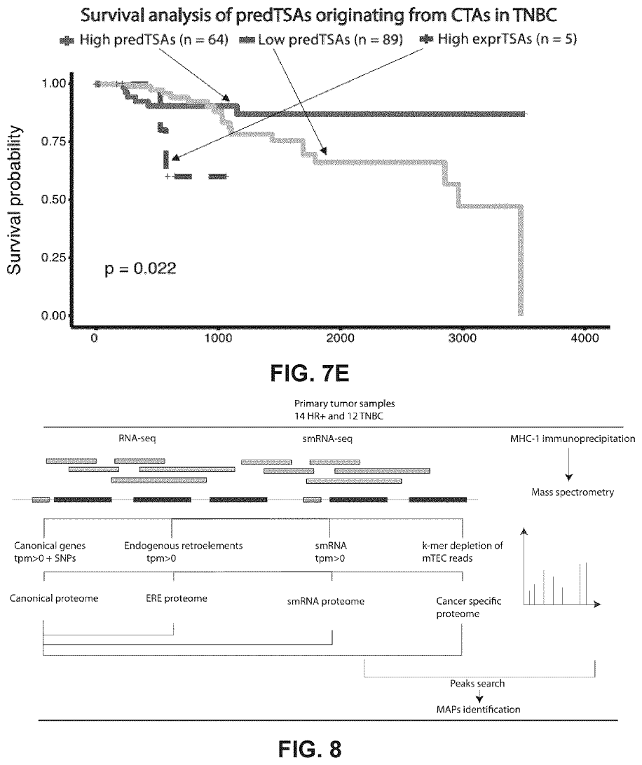

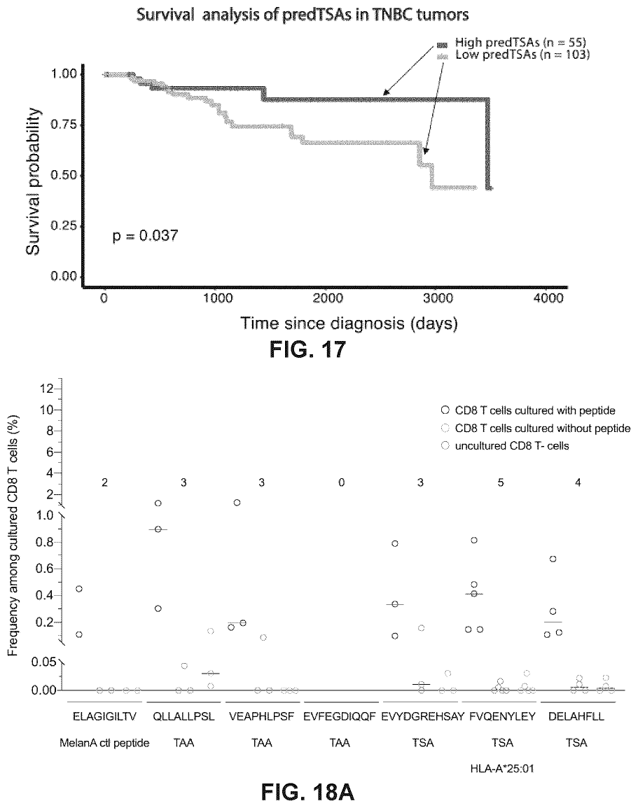

The number of presented TAAs had no impact on the survival of patients with HR⁺ or TNBC tumors (FIG. 7B, C). Likewise, the number of presented aeTSAs had no effect in patients with HR⁺ tumors (FIG. 7A), a group that expresses few aeTSAs (FIG. 4C). However, the presentation of more numerous aeTSAs correlated with better overall survival in the TNBC cohort (FIG. 17). This benefit was observed with aeTSAs deriving from both CTAs and noncoding regions (FIG. 7D, E). Notably, when only TSA expression was considered (and not the HLA allotypes), no differences were found between high and low expressors (FIG. 7D, E). This means that the favorable impact of aeTSAs presentation is HLA-restricted. Hence, it is due to MHC I presentation of the aeTSA peptide and not to the expression of the aeTSA-coding transcripts per se.

It may be concluded that aeTSAs reported herein confer an MHC class I-restricted survival advantage in patients with TNBC (FIG. 7D, E). This is not the case in patients with HR⁺ tumors (FIG. 7A), probably because they present fewer aeTSAs than TNBC tumors (FIG. 4C). Presentation of TAAs does not confer a similar survival advantage, likely because TAA expression is linked to activation of immunosuppressive pathways (FIG. 5C).

### Example 9: TSAs and TAAs Induce Expansion of Specific CD8 T Cells

It was next assessed whether TAAs and TSAs identified herein are able to induce CD8 T cell expansion ex vivo. This was assessed using the FEST (Functional Expansion of Specific T cells) assay that integrates T-cell receptor sequencing of short-term, peptide-stimulated cultures with a bioinformatic platform to identify antigen-specific clonotypic amplifications (Danilova et al., Cancer Immunol Res. 2018 August; 6(8):888-899. Epub 2018 Jun. 12, 2018.

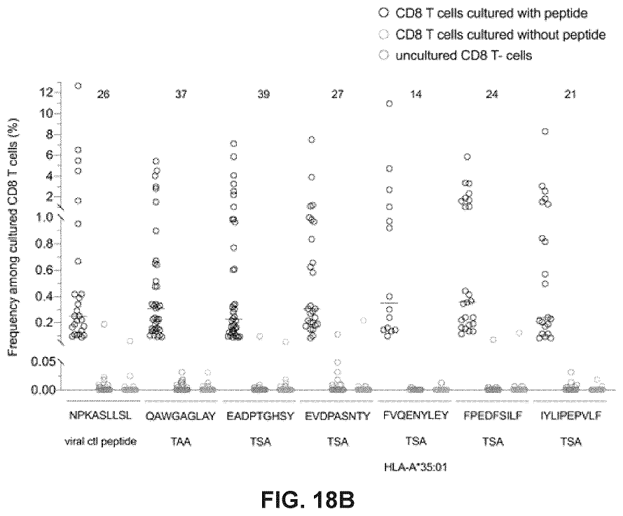

As shown in FIGS. 18A and 18B, significant antigen-specific CD8 T cell clonotype expansion was obtained for selected TSAs and TAAs in two different donors, providing compelling evidence that the TSAs and TAAs are immunogenic, and may be useful to stimulate an antitumor T cell response in subjects suffering from cancers expressing these TSAs and/or TAAs.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Sung H, Ferlay J, Siegel R L, Laversanne M, Soerjomataram I, Jemal A, et al. Global Cancer Statistics 2020: GLOBOCAN Estimates of Incidence and Mortality Worldwide for 36 Cancers in 185 Countries. CA: A Cancer Journal for Clinicians 2021; 71:209-49
2. Stanton S E, Disis M L. Clinical significance of tumor-infiltrating lymphocytes in breast cancer. J Immunother Cancer 2016; 4:59-
3. Emens L A. Breast Cancer Immunotherapy: Facts and Hopes. Clin Cancer Res 2018; 24:511-20
4. Denkert C, von Minckwitz G, Darb-Esfahani S, Lederer B, Heppner B I, Weber K E, et al. Tumour-infiltrating lymphocytes and prognosis in different subtypes of breast cancer: a pooled analysis of 3771 patients treated with neoadjuvant therapy. The Lancet Oncology 2018; 19:40-50
5. Schmid P, Adams S, Rugo H S, Schneeweiss A, Barrios C H, Iwata H, et al. Atezolizumab and Nab-Paclitaxel in Advanced Triple-Negative Breast Cancer. New England Journal of Medicine 2018; 379:2108-21
6. Goodman A M, Kato S, Bazhenova L, Patel S P, Frampton G M, Miller V, et al. Tumor Mutational Burden as an Independent Predictor of Response to Immunotherapy in Diverse Cancers. Mol Cancer Ther 2017; 16:2598-608
7. Barroso-Sousa R, Jain E, Cohen O, Kim D, Buendia-Buendia J, Winer E, et al. Prevalence and mutational determinants of high tumor mutation burden in breast cancer. Annals of Oncology 2020; 31:387-94
8. Ehx G E, Perreault C. Discovery and characterization of actionable tumor antigens. Genome Med 2019; 11:1-3
9. Bassani-Sternberg M, Braunlein E, Klar R, Engleitner T, Sinitcyn P, Audehm S, et al. Direct identification of clinically relevant neoepitopes presented on native human melanoma tissue by mass spectrometry. Nat Commun 2016; 7:13404
10. Loffler M W, Mohr C, Bichmann L, Freudenmann L K, Walzer M, Schroeder C M, et al. Multi-omics discovery of exome-derived neoantigens in hepatocellular carcinoma. Genome Med 2019; 11:28
11. Ehx G, Larouche J-D, Durette C, Laverdure J-P, Hesnard L, Vincent K, et al. Atypical acute myeloid leukemia-specific transcripts generate shared and immunogenic MHC class-I-associated epitopes. Immunity 2021; 54:737-52.e10
12. Yarmarkovich M, Marshall Q F, Warrington J M, Premaratne R, Farrel A, Groff D, et al. Cross-HLA targeting of intracellular oncoproteins with peptide-centric CARs. Nature 2021; 599:477-84
13. Laumont C M, Vincent K, Hesnard L, Audemard E, Bonneil E, Laverdure J-P, et al. Noncoding regions are the main source of targetable tumor-specific antigens. Science Translational Medicine 2018; 10:eaau5516
14. Zhao Q, Laverdure J-P, Lanoix J, Durette C, Côté C, Bonneil É, et al. Proteogenomics Uncovers a Vast Repertoire of Shared Tumor-Specific Antigens in Ovarian Cancer. Cancer Immunology Research 2020; 8:544-55
15. Chapuis A G, Egan D N, Bar M, Schmitt T M, McAfee M S, Paulson K G, et al. T cell receptor gene therapy targeting WT1 prevents acute myeloid leukemia relapse post-transplant. Nat Med 2019; 25:1064-72
16. Chiou S H, Tseng D, Reuben A, Mallajosyula V, Molina I S, Conley S, et al. Global analysis of shared T cell specificities in human non-small cell lung cancer enables HLA inference and antigen discovery. Immunity 2021; 54:586-602
17. Millar D G, Ohashi P S. Central tolerance: what you see is what you don't get! Nat Immunol 2016; 17:115-6
18. Marcu A, Bichmann L, Kuchenbecker L, Kowalewski D J, Freudenmann L K, Backert L, et al. HLA Ligand Atlas: a benign reference of HLA-presented peptides to improve T-cell-based cancer immunotherapy. Journal for ImmunoTherapy of Cancer 2021; 9:e002071
19. Sahin U, Oehm P, Derhovanessian E, Jabulowsky R A, Vormehr M, Gold M, et al. An RNA vaccine drives immunity in checkpoint-inhibitor-treated melanoma. Nature 2020; 585:107-12
20. Almeida L G, Sakabe N J, deOliveira A R, Silva M C, Mundstein A S, Cohen T, et al. CTdatabase: a knowledge-base of high-throughput and curated data on cancer-testis antigens. Nucleic Acids Res 2009; 37:D816-9
21. Yi X, Liao Y, Wen B, Li K, Dou Y, Savage S R, et al. caAtlas: An immunopeptidome atlas of human cancer. iScience 2021; 24:103107
22. Ternette N, Olde Nordkamp M J M, Müller J, Anderson A P, Nicastri A, Hill A V S, et al. Immunopeptidomic Profiling of HLA-A2-Positive Triple Negative Breast Cancer Identifies Potential Immunotherapy Target Antigens. Proteomics 2018; 18:e1700465-e
23. Larouche J-D, Trofimov A, Hesnard L, Ehx G, Zhao Q, Vincent K, et al. Widespread and tissue-specific expression of endogenous retroelements in human somatic tissues. Genome Medicine 2020; 12:40
24. Cleyle J, Hardy M P, Minati R, Courcelles M, Durette C, Lanoix J, et al. Immunopeptidomic analyses of colorectal cancers with and without microsatellite instability. Mol Cell Proteomics 2022; 21:100228
25. Reynisson B, Alvarez B, Paul S, Peters B, Nielsen M. NetMHCpan-4.1 and NetMHCIIpan-4.0: improved predictions of MHC antigen presentation by concurrent motif deconvolution and integration of MS MHC eluted ligand data. Nucleic Acids Res 2020; 48:W449-W54
26. Courcelles M, Durette C, Daouda T, Laverdure J P, Vincent K, Lemieux S, et al. MAPDP: a cloud-based computational platform for immunopeptidomics analyses. J Proteome Res 2020; 19:1873-81
27. Eng J K, Hoopmann M R, Jahan T A, Egertson J D, Noble W S, MacCoss M J. A deeper look into Comet—implementation and features. J Am Soc Mass Spectrom 2015; 26:1865-74
28. Szolek A, Schubert B, Mohr C, Sturm M, Feldhahn M, Kohlbacher O. OptiType: precision HLA typing from next-generation sequencing data. Bioinformatics 2014; 30:3310-6
29. Colaprico A, Silva T C, Olsen C, Garofano L, Cava C, Garolini D, et al. TCGAbiolinks: an R/Bioconductor package for integrative analysis of TCGA data. Nucleic Acids Res 2016; 44:e71
30. Qian J, Olbrecht S, Boeckx B, Vos H, Laoui D, Etlioglu E, et al. A pan-cancer blueprint of the heterogeneous tumor microenvironment revealed by single-cell profiling. Cell Res 2020; 30:745-62
31. Satija R, Farrell J A, Gennert D, Schier A F, Regev A. Spatial reconstruction of single-cell gene expression data. Nat Biotechnol 2015; 33:495-502
32. Ritchie M E, Phipson B, Wu D, Hu Y, Law C W, Shi W, et al. limma powers differential expression analyses for RNA-sequencing and microarray studies. Nucleic Acids Res 2015; 43:e47
33. Korotkevich G, Sukhov V, Budin N, Shpak B, Artyomov M N, Sergushichev A. Fast gene set enrichment analysis. 2021:060012
34. Pearson H, Daouda T, Granados D P, Durette C, Bonneil E, Courcelles M, et al. MHC class I-associated peptides derive from selective regions of the human genome. The Journal of Clinical Investigation 2016; 126:4690-701
35. Caron E, Vincent K, Fortier M-H, Laverdure J-P, Bramoullé A, Hardy M-P, et al. The MHC I immunopeptidome conveys to the cell surface an integrative view of cellular regulation. Mol Syst Biol 2011; 7:533-
36. Giussani M, Merlino G, Cappelletti V, Tagliabue E, Daidone M G. Tumor-extracellular matrix interactions: Identification of tools associated with breast cancer progression. Seminars in Cancer Biology 2015; 35:3-10
37. Wu Y H, Huang Y F, Chang T H, Chen C C, Wu P Y, Huang S C, et al. COL11A1 activates cancer-associated fibroblasts by modulating TGF-beta3 through the NF-kappaB/IGFBP2 axis in ovarian cancer cells. Oncogene 2021; 40:4503-19
38. Sato F, Sagara A, Tajima K, Miura S, Inaba K, Ando Y, et al. COL8A1 facilitates the growth of triple-negative breast cancer via FAK/Src activation. Breast Cancer Res Treat 2022
39. Wang-Johanning F, Li M, Esteva F J, Hess K R, Yin B, Rycaj K, et al. Human endogenous retrovirus type K antibodies and mRNA as serum biomarkers of early-stage breast cancer. Int J Cancer 2014; 134:587-95
40. Mulrane L, McGee S F, Gallagher W M, O'Connor D P. miRNA dysregulation in breast cancer. Cancer Res 2013; 73:6554-62
41. Muller M, Gfeller D, Coukos G, Bassani-Sternberg M. ‘Hotspots’ of Antigen Presentation Revealed by Human Leukocyte Antigen Ligandomics for Neoantigen Prioritization. Front Immunol 2017; 8:1367
42. Sender R, Milo R. The distribution of cellular turnover in the human body. Nat Med 2021; 27:45-8
43. Danaher P, Warren S, Dennis L, D'Amico L, White A, Disis M L, et al. Gene expression markers of Tumor Infiltrating Leukocytes. J Immunother Cancer 2017; 5:18
44. Kobayashi Y, Lim S-O, Yamaguchi H. Oncogenic signaling pathways associated with immune evasion and resistance to immune checkpoint inhibitors in cancer. Seminars in Cancer Biology 2020; 65:51-64
45. Subramanian A, Tamayo P, Mootha V K, Mukherjee S, Ebert B L, Gillette M A, et al. Gene set enrichment analysis: A knowledge-based approach for interpreting genome-wide expression profiles. Proceedings of the National Academy of Sciences 2005; 102:15545-50
46. Zhang Z, Richmond A. The Role of PI3K Inhibition in the Treatment of Breast Cancer, Alone or Combined With Immune Checkpoint Inhibitors. Front Mol Biosci 2021; 8:648663
47. Zhu K, Cai L, Cui C, de Los Toyos J R, Anastassiou D. Single-cell analysis reveals the pan-cancer invasiveness-associated transition of adipose-derived stromal cells into COL11A1-expressing cancer-associated fibroblasts. PLoS Comput Biol 2021; 17:e1009228
48. Nunna V, Jalal N, Bureik M. Anti-CYP4Z1 autoantibodies detected in breast cancer patients. Cell Mol Immunol 2017; 14:572-4
49. Su X, Xu Y, Fox G C, Xiang J, Kwakwa K A, Davis J L, et al. Breast cancer-derived GM-CSF regulates arginase 1 in myeloid cells to promote an immunosuppressive microenvironment. J Clin Invest 2021; 131
50. Spranger S, Gajewski T F. Impact of oncogenic pathways on evasion of antitumour immune responses. Nat Rev Cancer 2018; 18:139-47
51. Carnevalli L S, Sinclair C, Taylor M A, Gutierrez P M, Langdon S, Coenen-Stass A M L, et al. PI3Kalpha/delta inhibition promotes anti-tumor immunity through direct enhancement of effector CD8(+) T-cell activity. J Immunother Cancer 2018; 6:158
52. Buechler M B, Fu W, Turley S J. Fibroblast-macrophage reciprocal interactions in health, fibrosis, and cancer. Immunity 2021; 54:903-15
53. Zhang Y, Chertov O, Zhang J, Hassan R, Pastan I. Cytotoxic activity of immunotoxin SS1P is modulated by TACE-dependent mesothelin shedding. Cancer Res 2011; 71:5915-22

1. A tumor antigen peptide (TAP) comprising or consisting of any one of the amino acid sequences set forth in SEQ ID NOs: 4, 1-3 and 5-35.

2. The TAP of claim 1, wherein the TAP binds to (i) an HLA-A*02:01 molecule and comprises or consists of the sequence of SEQ ID NO: 22; (ii) an HLA-A*03:01 molecule and comprises or consists of the sequence of SEQ ID NO: 19; (iii) an HLA-A*11:01 molecule and comprises or consists of the sequence of SEQ ID NO: 1, 17 or 28; (iv) an HLA-A*24:02 molecule and comprises or consists of the sequence of SEQ ID NO: 6 or 30; (v) an HLA-A*25:01 molecule and comprises or consists of the sequence of SEQ ID NO: 10; (vi) an HLA-A*26:01 molecule and comprises or consists of the sequence of SEQ ID NO: 15; (vii) an HLA-A*31:01 molecule and comprises or consists of the sequence of SEQ ID NO: 8, 9 or 29; (viii) an HLA-A*33:01 molecule and comprises or consists of the sequence of SEQ ID NO: 2 or 3; (ix) an HLA-B*15:01 molecule and comprises or consists of the sequence of SEQ ID NO: 26; (x) an HLA-B*18:01 molecule and comprises or consists of the sequence of SEQ ID NO: 13, 14 or 33; (xi) an HLA-B*27:05 molecule and comprises or consists of the sequence of SEQ ID NO: 27; (xii) an HLA-B*35:01 molecule and comprises or consists of the sequence of SEQ ID NO: 4, 12 or 23; (xiii) an HLA-B*35:03 molecule and comprises or consists of the sequence of SEQ ID NO: 38; (xiv) an HLA-B*38:01 molecule and comprises or consists of the sequence of SEQ ID NO: 34; (xv) an HLA-B*40:01 molecule and comprises or consists of the sequence of SEQ ID NO: 20; (xvi) an HLA-B*49:01 molecule and comprises or consists of the sequence of SEQ ID NO: 11 or 24; (xvii) an HLA-B*50:01 molecule and comprises or consists of the sequence of SEQ ID NO: 5 or 7; (xviii) an HLA-B*51:01 molecule and comprises or consists of the sequence of SEQ ID NO: 35 or 37; (xix) an HLA-B*52:01 molecule and comprises or consists of the sequence of SEQ ID NO: 18; (xx) an HLA-B*58:01 molecule and comprises or consists of the sequence of SEQ ID NO: 36; (xxi) an HLA-C*01:02 molecule and comprises or consists of the sequence of SEQ ID NO: 25; or (xxii) an HLA-C*12:03 molecule and comprises or consists of the sequence of SEQ ID NO: 16 or 21.

3.-23. (canceled)

24. The TAP of claim 1, which is encoded by a sequence located a non-protein coding region of the genome.

25. The TAP of claim 24, wherein said non-protein coding region of the genome is an intergenic region or a long non-coding RNA.

26. (canceled)

27. A combination comprising at least two of the TAPs defined in claim 1.

28.-30. (canceled)

31. A synthetic long peptide (SLP) comprising at least one of the amino acid sequences defined in claim 1.

32. A synthetic long peptide (SLP) comprising at least 5, 10, 15 or 20 of the amino acid sequences defined in claim 1.

33. A vesicle or particle comprising (a) the TAP of claim 1, (b) a synthetic long peptide (SLP) comprising at least one of the amino acid sequences defined in claim 1, (c) a nucleic acid encoding (i) at least one of the TAP defined in claim 1, or (ii) a synthetic long peptide (SLP) comprising at least one of the amino acid sequences defined in claim 1.

34. The vesicle or particle of claim 33, which is a lipid nanoparticle (LNP).

35. (canceled)

36. A composition comprising (a) the TAP of claim 1, (b) a combination comprising at least two of the TAPs defined in claim 1, (c) a synthetic long peptide (SLP) comprising at least one of the amino acid sequences defined in claim 1, or (d) a nucleic acid encoding the TAP defined in (a), (ii) the combination defined in (b), or (iii) the SLP defined in (c), and a pharmaceutically acceptable carrier.

37. A vaccine comprising (a) the TAP of claim 1, (b) a combination comprising at least two of the TAPs defined in claim 1, (c) a synthetic long peptide (SLP) comprising at least one of the amino acid sequences defined in claim 1, (d) a nucleic acid encoding the TAP defined in (a), (ii) the combination defined in (b), or (iii) the SLP defined in (c), and an adjuvant.

38.-41. (canceled)

42. An isolated cell expressing at its surface major histocompatibility complex (MHC) class I molecules comprising the TAP of claim 1 or any combination thereof in their peptide binding groove.

43. The cell of claim 42, which is an antigen-presenting cell (APC).

44. (canceled)

45. A T-cell receptor (TCR) or an antibody or an antigen-binding fragment thereof that specifically recognizes MHC class I molecules expressed at the surface of the cell of claim 42.

46.-51. (canceled)

52. A chimeric antigen receptor (CAR) comprising the antibody or an antigen-binding fragment thereof of claim 45.

53. An isolated cell expressing at its cell surface the TCR of claim 45 or a chimeric antigen receptor (CAR) comprising the antibody or an antigen-binding fragment thereof of claim 45.

54. (canceled)

55. A cell population comprising at least 0.5% or 1% of the isolated cell as defined in claim 53.

56. A method of treating breast cancer in a subject comprising administering to the subject an effective amount of:
(a) a TAP comprising or consisting of any one of the sequences set forth in SEQ ID NOs: 1-61 or any combination thereof, or a synthetic long peptide (SLP) comprising at least one of the sequences set forth in SEQ ID NOs: 1-61;
(b) at least one nucleic acid encoding the TAP, combination thereof or SLP defined in (a);
(c) a vesicle or particle comprising the TAP, combination thereof or SLP defined in (a) or the at least one nucleic acid defined in (b);
(d) a composition comprising the TAP, combination thereof or SLP defined in (a), the at least one nucleic acid defined in (b), or the vesicle or particle defined in (c), and a pharmaceutically acceptable carrier;
(e) a vaccine comprising the TAP, combination thereof or SLP defined in (a), the at least one nucleic acid defined in (b), the vesicle or particle defined in (c), or the composition defined in (d), and an adjuvant;
(f) a cell expressing at its surface major histocompatibility complex (MHC) class I molecules comprising the TAP or combination thereof defined in (a) in their peptide binding groove;
(g) a cell expressing at its cell surface a T-cell receptor (TCR) that specifically recognizes MHC class I molecules expressed at the surface of the cell defined in (f); or
(h) a soluble TCR, an antibody or an antigen-binding fragment thereof that specifically binds to the MHC class I molecules expressed at the surface of the cell defined in (f).

57.-67. (canceled)

68. A nucleic acid encoding (i) at least one of the TAP defined in claim 1, (ii) a combination comprising at least two of the TAPs defined in claim 1, or (iii) a synthetic long peptide (SLP) comprising at least one of the amino acid sequences defined in claim 1.

69. The nucleic acid of claim 68, which is an mRNA.
